(19) United States
(12) Patent Application Publication
(10) Pub. No.: US 2020/0054796 A1
(43) Pub. Date: Feb. 20, 2020

(54) Silk Fibroin Tracheal Stent

(71) Applicants: Trustees of Tufts College, Medford, MA (US); Massachusetts Eye and Ear Infirmary, Boston, MA (US); The General Hospital Corporation, Boston, MA (US)

(72) Inventors: David L. Kaplan, Concord, MA (US); Michael Whalen, Needham, MA (US); Christopher Hartnick, Newton, MA (US); Meghan McGill, Somerville, MA (US)

(21) Appl. No.: 16/487,563

(22) PCT Filed: Feb. 21, 2018
(86) PCT No.: PCT/US2018/018998

§ 371 Date: Aug. 21, 2019

Related U.S. Application Data
(60) Provisional application No. 62/461,552, filed on Feb. 21, 2017.

(51) Int. Cl. A61L 31/00; A61F 2/04; A61L 31/14; A61L 31/12; A61L 31/16; A61L 31/04; A61L 31/02; A61F 2/93; B29D 23/00

(52) U.S. Cl. CPC A61L 31/005; B29K 2105/0073; A61L 31/141; A61L 31/129; A61L 31/146; A61L 31/16; A61L 31/047; A61L 31/148; A61L 31/128; A61L 31/022; A61F 2/93; B29D 23/00; A61F 2002/046; A61F 2220/0016; A61F 2240/001; A61L 2430/22; A61F 2230/0069; A61F 2/04

(57) ABSTRACT

Bioresorbable silk fibroin tracheal stents can be designed and engineered to maintain a tracheal opening. A tracheal stent will maintain a tracheal opening for a period while tissue structure and function is restored. Bioresorbable silk fibroin tracheal stents programmably degrade without negative biological or clinical outcomes. Bioresorbable silk fibroin tracheal stents do not need to be removed following tracheal restoration. Bioresorbable biopolymer tracheal stents can be internally or externally deployed. Bioresorbable biopolymer tracheal stents, for example can be internally or externally deployed in a patient. Such stents may be affixed to function as a splint with tunable mechanically properties to treat, for example, a patient with severe airway collapse.

### CROSS REFERENCE TO RELATED APPLICATIONS

This patent application claims the benefit of priority of U.S. application No. 62/461,552 filed on Feb. 21, 2017, the contents of which are hereby incorporated by reference in their entirety for all purposes herein.

### BACKGROUND

Stents have been applied where an immediate mechanical structure is necessary maintain an opening, improve patency of a mechanical opening, or prevent restenosis. Stents have been widely employed for vessel openings. Recently, both inter- and extraluminal tracheal stents have been utilized to treat tracheal collapse following prolonged tracheostomy tube placement or tracheal surgery to treat severe tracheomalacia.

### SUMMARY

The present disclosure provides, among other things, tracheal stents. Provided tracheal stents are useful, for example, to support a tracheal wall and prevent tracheal collapse. In some embodiments, provided tracheal stents biocompatible, biodegradable, bioresorbable, cytocompatible, and able to stabilize biologically labile compounds, such as enzymes as well as other additives, agents, and/or functional moieties. In some embodiments, provided tracheal stents degrade and reabsorb into the body over a specified period after tracheal support is no longer needed. The present disclosure also provides methods of preparing and systems for deploying such stents.

Implementations of the present disclosure are useful for applications, including but not limited to: treatment for tracheal collapse, for example due to suprastomal collapse, tracheal stenosis, or tracheomalacia. In some embodiments, applications, including for treatment following prolonged tracheostomy tube placement or tracheal surgery to treat severe tracheomalacia. In particular, the present disclosure discloses embodiments for pediatric treatment.

In some embodiments, the present disclosure provides tracheal stent grafts that are made of a bioresorbable biopolymer. In some embodiments, provided tracheal stent grafts are flexible biomaterials that characterized by physical and mechanical properties that are compatible to human tracheal tissues.

In some embodiments, provided tracheal stent grafts are or include polymers or proteins. In some embodiments, polymers or proteins are natural or synthetic. In some embodiments, polymers or proteins are or include agarose, alginate, amyloid, cellulose, chitin, chitosan, collagen, elastin, gelatin, keratin, hyaluronic acid, polydimethylsiloxane, poly(ethylene glycol), poly(propylene glycol), polyhydroxyalkanoates, poly(lactide-co-glycolide), poly(methyl methacrylate), poly(vinyl-alcohol) (PVA), pullulan, resilin, silk, starch, or combinations thereof.

In some embodiments, provided tracheal stent grafts are made of or include silk. In some embodiments, provided tracheal stent grafts are made of or include silk fibroin based. In some embodiments, provided tracheal stent grafts are made of or include other natural or synthetic polymers or proteins.

In some embodiments, provided tracheal stent graphs are made of or include silk fibroin characterized by beta-sheet secondary structure.

In some embodiments, provided silk fibroin based tracheal stent graphs are porous.

In some embodiments, provided silk fibroin based tracheal stent graphs are substantially cylindrical.

In some embodiments, provided silk fibroin based tracheal stent graphs are characterized by dimensions that are adjustable to accommodate any size airway. In some embodiments, provided silk fibroin based tracheal stent graphs are sized for infants. In some embodiments, provided silk fibroin based tracheal stent graphs are sized for pediatric patients. In some embodiments, provided silk fibroin based tracheal stent graphs are sized for adult patients.

In some embodiments, provided silk fibroin based tracheal stent graphs are characterized by a radius of about 2.5 mm to about 10 mm.

In some embodiments, provided tracheal stent graphs are about 0.5 cm to about 8 cm in length.

In some embodiments, provided tracheal stent graphs include walls that are about 1 mm to about 5 mm thick.

In some embodiments, provided silk fibroin based tracheal stent graphs accommodate external tracheal diameters of about 6 mm to about 14 mm.

In some embodiments, provided tracheal stent graphs include a radial opening between about 0° and about 240°. In some embodiments, provided tracheal stent graphs are substantially cylindrical and include a radial opening between about 0° and about 240°.

In some embodiments, provided silk fibroin based tracheal stent graphs have tunable mechanical properties. In some embodiments, provided silk fibroin based tracheal stent graphs have been developed as scaffolds with control, manipulation, and tailoring cellular processes and integration.

In some embodiments, provided silk fibroin based tracheal stent graphs have an average radial strength of about 1 mmHg to about 1000 mm Hg.

In some embodiments, provided silk fibroin based tracheal stent graphs have a tensile strength of about 0.05 MPa to 30 MPa.

In some embodiments, provided silk fibroin based tracheal stent graphs have a mechanical stiffness of about 0.5 kN/m to about 250 kN/m.

In some embodiments, provided silk fibroin based tracheal stent graphs are characterized in that when they are implanted, they do not produce an inflammatory tissue response.

In some embodiments, provided silk fibroin based tracheal stent graphs are non-toxic. In some embodiments, provided silk fibroin based tracheal stent graphs are fully bioresorbable upon degradation.

In some embodiments, provided silk fibroin based tracheal stent graphs predictably degrade over a period. In some embodiments, provided silk fibroin based tracheal stent graphs predictably degrade when exposed to amino acids or enzymes present in body cells. In some embodiments, provided silk fibroin based tracheal stent graphs degrade with tunable target lifetimes. In some embodiments, provided silk fibroin based tracheal stent graphs degrade in vivo after about 3 months to about 2 years.

In some embodiments, provided silk fibroin based tracheal stent graphs degrade and will progressively disappear after tissue remodeling. In some embodiments, provided silk fibroin based tracheal stent graphs degrade after tissue remodeling and so that they do not need to be excised. Degradation and reabsorption is particularly useful in cases where a subject's tissue outgrows a diameter of provided silk fibroin based tracheal stents, for example when a subject is a child or adolescent suffering from congenital disease or injury.

In some embodiments, such tracheal stent grafts are designed and engineered to be externally affixed or grafted to an anterior tracheal wall of a subject.

In some embodiments, provided silk fibroin based tracheal stent graphs are suturable. In some embodiments, provided silk fibroin based tracheal stent graphs are capable of fixation onto an external aspect of a subject's trachea. In some embodiments, provided silk fibroin based tracheal stent graphs are designed and constructed with holes to receive sutures.

In some embodiments, provided silk fibroin based tracheal stent graphs are designed and constructed with barbs on its outer surface. In some embodiments, barbs prevent migration of provided silk fibroin based tracheal stent graphs after deployment.

In some embodiments, provided silk fibroin based tracheal stents are laser cut or designed to be laser cut.

In some embodiments, provided silk fibroin based tracheal stent graphs allow for both bolstering and application upwards and outwards to promote a greatest tracheal diameter.

In some embodiments, provided silk fibroin based tracheal stents are or include silk fibroin made from a solution having a silk fibroin concentration of about 2% to about 40% silk. In some embodiments, provided silk fibroin based tracheal stents are or include silk fibroin made from a solution having that is about 20% (w/w) to about 40% (w/w) silk fibroin.

In some embodiments, provided silk fibroin based tracheal stents are reinforced.

In some embodiments, provided silk fibroin based tracheal stents are or include silk fibroin fibers. In some embodiments, silk fibroin fibers are added to provide stability and/or to reinforce provided silk fibroin based tracheal stents.

In some embodiments, provided silk fibroin based tracheal stent graphs include a plurality of layers of a silk fibroin material. In some embodiments, a plurality of layers provides reinforcement. In some embodiments, layers of a plurality of layers include silk fibers to add stability and/or to reinforce provided silk fibroin based tracheal stents.

In some embodiments, provided silk fibroin based tracheal stent graphs further include a stiff silk film layer. In some embodiments, a stiff silk film layer reinforces a tracheal stent graph.

In some embodiments, a stiff fiber reinforced silk film layer is a mesh layer. In some embodiments, a stiff fiber reinforced is silk fibroin fibers, concentrated silk depositions, other polymer materials.

In some embodiments, a reinforced layer is a silk film. In some embodiments, a reinforced layer is a silk film with other polymers or metals. In some embodiments, metal reinforcements are or include magnesium.

In some embodiments, provided silk fibroin based tracheal stent graphs include struts positioned on or within a silk fibroin material. In some embodiments, struts provide reinforcement.

In some embodiments, provided silk fibroin based tracheal stent graphs include stiff struts positioned on or within a flexible silk fibroin material. In some embodiments, a flexible silk fibroin material is a porous flexible silk fibroin scaffold.

In some embodiments, struts are or include silk fibroin fibers, concentrated silk depositions, other polymer materials, or metals. In some embodiments, metal struts are or include magnesium.

In some embodiments, provided silk fibroin based tracheal stent graphs are characterized in that they are capable of incorporating additives, agents, or functional moieties. In some embodiments, provided silk fibroin based tracheal stent graphs are coated with additives, agents, or functional moieties. In some embodiments, provided silk fibroin based tracheal stent graphs are embedded with additives, agents, or functional moieties.

In some embodiments, additives, agents, or functional moieties include a plasticizer. In some embodiments, a plasticizer is or includes glycerol.

In some embodiments, a plasticizer is or includes 1,2-butylene glycol; 2-amino-2-methyl-1,3-propanediol; 2,3-butylene glycol; allyl glycolate; butyl lactate; diethanolamine; diethylene glycol monoethyl ether; ethyl glycolate; ethyl lactate; ethylene glycol; ethylene glycol monoethyl ether; glycerol; glyceryl monostearate; monoethanolamine; monisopropanolamine; monopropylene glycol monoisopropyl ether; polyethylene glycol; polyethylene oxides; propylene glycol; propylene glycol monoethyl ether; sorbitol lactate; styrene glycol; triethanolamine; triethylenetetramine; or combinations thereof.

In some embodiments, provided silk fibroin based tracheal stents are or include a plasticizer having a concentration of up to about 50% by weight of such a tracheal stent. In some embodiments, provided silk fibroin based tracheal stents are or include a plasticizer having a concentration of about 1% to about 50% by weight of such a tracheal stent. In some embodiments, provided silk fibroin based tracheal stents are or include a plasticizer having a concentration of about 5% to about 30% by weight of such a tracheal stent.

In some embodiments, provided silk fibroin based tracheal stents are a blend of silk fibroin and a plasticizer having a silk fibroin to plasticizer ratio of about 1000:1 to about 1:1 by dry weight.

In some embodiments, additives, agents, or functional moieties include active agents, alcohols; antibodies or fragments or portions thereof; antibodies, antibiotics or antimicrobial compounds; antigens or epitopes; anti-proliferative agents; aptamers; biologically or pharmaceutically active compounds; biopolymers; cells; cell adhesion proteins; cell attachment mediators; cleavable cross-linkers; cytokines; DNA, enzymes; glycogens or other sugars; growth factors or recombinant growth factors and fragments and variants thereof; hormone antagonists; hormones; modified RNA/protein composites, nanoparticles; nucleic acid analogs; nucleic acids; nucleotides; oligonucleotides; peptide nucleic acids (PNA); peptides; plasticizer, proteins; radiopaque markers; RNA; small molecules; soluble drugs, therapeutic agents and prodrugs; toxins; or combinations thereof.

In some embodiments, cells are viable cells. In some embodiments, viable cells are subject derived cells.

In some embodiments, silk fibroin based tracheal stent graphs are drug-eluting stents. In some embodiments, silk fibroin based tracheal stent graphs are designed and engineered to deliver drug payloads over long time. In some embodiments, an ability to deliver drug payloads over long periods limits or reduces an occurrence of localized restenosis when an implant is resorbed.

In some embodiments, silk fibroin based tracheal stent graphs are designed and engineered to allow segregation of different drugs throughout its bulk material, yielding a complex drug release profile. In some embodiments, such an approach also presents a unique opportunity to locally deliver multiple drugs over several time scales to treat a variety of clinical conditions.

In some embodiments, provided silk fibroin based tracheal stent graphs are coated with additives, agents, or functional moieties including topical treatments.

In some embodiments, provided silk fibroin based tracheal stent graphs are characterized in that they can be sterilized via autoclaving. In some embodiments, provided silk fibroin based tracheal stent graphs are characterized in that they can be sterilized using ethylene oxide. In some embodiments, provided silk fibroin based tracheal stent graphs are characterized in that they can be sterilized using gamma irradiation. In some embodiments, provided silk fibroin based tracheal stent graphs are characterized in that they can be sterilized using a peroxide.

In some embodiments, provided silk fibroin based tracheal stent graphs are characterized in that they are shelf stable for a period of years.

In some embodiments, provided silk fibroin based tracheal stent graphs facilitate more precise diagnostic interpretation using computed tomography, magnetic resonance imaging or radiopaque markers.

In some embodiments, methods of manufacturing silk fibroin based tracheal stent graphs are provided. In some embodiments, provided methods of manufacturing include providing a silk fibroin solution. In some embodiments, provided silk fibroin solutions have a concentration of about 2% to about 40%.

In some embodiments, provided methods of manufacturing include adding a silk fibroin solution to a mold.

In some embodiments, provided methods of manufacturing include freezing a silk fibroin solution to form a tracheal stent. In some embodiments, provided methods of manufacturing include porogen leaching a silk fibroin solution to form a tracheal stent. In some embodiments, provided methods of manufacturing include gel spinning a silk fibroin solution to form a tracheal stent. In some embodiments, provided methods of manufacturing include micromolding a silk fibroin solution to form a tracheal stent.

In some embodiments, a step of freezing includes lowering a temperature of the solution to about −45° C. at a rate of about 0.1° C./minute to about 5° C./minute. In some embodiments a step of freezing includes drying a silk fibroin solution under vacuum.

In some embodiments, methods further include a step of submerging a tracheal stent in methanol.

In some embodiments, methods further include a step of water annealing a tracheal stent.

In some embodiments, methods include a step of encapsulating or embedding an additive, agent or functional moiety a provided silk fibroin tracheal stent. In some embodiments, a step of encapsulating or embedding includes blending or mixing an additive, agent or functional moiety in a silk fibroin solution. In some embodiments, methods include a step of coating an additive, agent or functional moiety on a surface of a provided silk fibroin tracheal stent.

In some embodiments, an additive, agent, or functional moiety is or includes an active agent, a plasticizer, silk fibroin fibers, a therapeutic, or combinations thereof. In some embodiments, a plasticizer is or includes 1,2-butylene glycol; 2-amino-2-methyl-1,3-propanediol; 2,3-butylene glycol; allyl glycolate; butyl lactate; diethanolamine; diethylene glycol monoethyl ether; ethyl glycolate; ethyl lactate; ethylene glycol; ethylene glycol monoethyl ether; glycerol; glyceryl monostearate; monoethanolamine; monisopropanolamine; monopropylene glycol monoisopropyl ether; polyethylene glycol; polyethylene oxides; propylene glycol; propylene glycol monoethyl ether; sorbitol lactate; styrene glycol; triethanolamine; triethylenetetramine; or combinations thereof.

In some embodiments, additives, agents, or functional moieties are or include antibodies or fragments or portions thereof antibiotics or antimicrobial compounds; antigens or epitopes; anti-proliferative agents; aptamers; biopolymers; cell adhesion proteins, cell attachment mediators; cleavable cross-linkers; cytokines; enzymes; growth factors or recombinant growth factors and fragments and variants thereof; hormone antagonists; hormones; nanoparticles; nucleic acid analogs; nucleic acids; nucleotides; oligonucleotides; peptide nucleic acids (PNA); peptides; proteins; radiopaque markers; small molecules; soluble drugs, therapeutic agents and prodrugs; toxins; or combinations thereof.

In some embodiments, additives, agents, or functional moieties are or include cells. In some embodiments, cells are viable cells. In some embodiments, viable cells are cells derived from a subject. In some embodiments, methods include a step of encapsulating or embedding viable cells. In some embodiments, encapsulating or embedding includes blending or mixing viable cells with a silk fibroin solution.

In some embodiments, provided methods of manufacturing a silk fibroin based tracheal stent graph include passing a silk fibroin solution through a 3D printer to generate a tracheal stent graph.

In some embodiments, provided methods of manufacturing a silk fibroin based tracheal stent graph include machine cutting or laser cutting stent design.

In some embodiments, provided methods of manufacturing a silk fibroin based tracheal stent graph include machine cutting or laser cutting a stent radial opening.

In some embodiments, methods of deploying silk fibroin based tracheal stent graphs are provided. In some embodiments, methods of deploying include grafting a silk fibroin based tracheal stent graph to an external site of a subject's trachea. In some embodiments, methods of deploying a silk fibroin based tracheal stent graph include externally affixing it to a tracheal wall for treatment of suprasomal collapse, tracheal malacia, or tracheal stenosis.

In some embodiments, methods further include a step of sterilizing a tracheal stent. In some embodiments, sterilizing is preformed via autoclave, ethylene oxide, gamma irradiation and/or peroxide.

In some embodiments, methods of deploying include ratcheting of provided silk fibroin based tracheal stents. In some embodiments, methods of deploying provided silk fibroin based tracheal stents include a ratcheting design for increasing stent diameter or radius.

In some embodiments, ratcheted designs or designs with a larger radius are useful to accommodate nerves.

These and other capabilities of the disclosure, along with the disclosure itself, will be more fully understood after a review of the following figures, detailed description, and claims.

### BRIEF DESCRIPTION OF THE DRAWING

The foregoing and other objects, aspects, features, and advantages of the present disclosure will become more apparent and better understood by referring to the following description taken in conjunction with the accompanying figures in which:

FIG. 1 shows a ratcheting tracheal stent and design. FIGS. 1A-1F show a ratcheting polymer stent according to a first embodiment of the invention.

FIG. 2 shows a second ratcheting tracheal stent and design. FIGS. 2A-2E show a ratcheting polymer stent according to a second embodiment of the invention.

FIG. 3 shows a third ratcheting tracheal stent and design. FIGS. 3A-3C show a ratcheting polymer stent according to a third embodiment of the invention.

FIG. 4 shows a fourth ratcheting tracheal stent and design. FIGS. 4A-4C show a ratcheting polymer stent according to a fourth embodiment of the invention.

FIG. 5 shows flexible and reinforced tracheal stents. FIGS. 5A-5C show a flexible and reinforced stent according to a fifth embodiment of the invention.

FIG. 6 shows a reinforced tracheal stent.

FIG. 7 shows a tracheal stent at 3 month explant in preclinical rabbit model.

FIG. 8 shows a flexible tracheal stent design.

FIG. 9 shows a fabrication method for silk fibroin tracheal splints from silk cocoons to 180° porous, flexible stent with a reinforced silk coating as disclosed in some embodiments herein.

FIG. 10 shows a tracheal ring resection and splint implantation. FIG. 10 at panel A shows the trachea is exposed via a vertical incision, and the overlying skin, muscle, and fascia is gently retracted laterally. FIG. 10 at panel B shows under a sterile technique, the tracheal rings are carefully dissected from the mucosa to induce airway malacia. FIG. 10 at panel C shows the splint is applied over the area of tracheomalacia and sutured into place. FIG. 10 at panel D shows the surgical incision is closed with a rubber band drain left in place.

FIG. 11 shows an example of a surgically-induced tracheomalacia in a rabbit airway prior to implantation of the bioresorbable silk fibroin splint. FIG. 11 at panel A shows maximal lumen size with tidal expiration. FIG. 11 at panel B shows minimum lumen size during spontaneous inhalation.

FIG. 12 shows a suture and testing. FIG. 12 at pane Ai shows a suture inserted into a rectangular sample of a silk fibroin splint. FIG. 12 at panel Aii shows a suture stressed to failure demonstrating that a suture can be inserted and resist a substantial force (1.8±0.5 N, N=3). FIG. 12 at panel B shows a loss in the maximum force (%) vs. loss in mass (%) of silk fibroin splints incubated in a protease solution at 37° C. over 6 weeks to mimic in vivo degradation. Maximum force was obtained from cyclic compression testing of hydrated stents, and is reported as an average and standard deviation of N=4 samples. FIG. 12 at Panel Ci shows a scanning electron microscopy (SEM) image at Day 0. FIG. 12 at Panel Cii shows a SEM image at Week 6 exhibiting evidence of degradation on the surface of the splints.

FIG. 13 shows tracheal dynamic change as measured using an image-based assay.

FIG. 14 shows the histology of the rabbit trachea, at time of resection.

### DEFINITIONS

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Unless otherwise stated, the terms “about” and “approximately” may be understood to permit standard variation as would be understood by those of ordinary skill in the art. Where ranges are provided herein, the endpoints are included. As used in this application, the term “comprise” and variations of the term, such as “comprising” and “comprises,” are not intended to exclude other additives, components, integers or steps.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

“Amino acid”: As used herein, the term “amino acid,” in its broadest sense, refers to any compound and/or substance that can be incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, and/or substitution as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” is used to refer to a free amino acid; in some embodiments it is used to refer to an amino acid residue of a polypeptide.

“Antibody”: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y′s stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. Amino acid sequence comparisons among antibody polypeptide chains have defined two light chain (κ and λ) classes, several heavy chain (e.g., μ, γ, α, ε, δ) classes, and certain heavy chain subclasses (α1, α2, γ1, γ2, γ3, and γ4). Antibody classes (IgA [including IgA1, IgA2], IgD, IgE, IgG [including IgG1, IgG2, IgG3, IgG4], IgM) are defined based on the class of the utilized heavy chain sequences. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is monoclonal; in some embodiments, an antibody is monoclonal. In some embodiments, an antibody has constant region sequences that are characteristic of mouse, rabbit, primate, or human antibodies. In some embodiments, an antibody sequence elements are humanized, primatized, chimeric, etc., as is known in the art. Moreover, the term “antibody” as used herein, will be understood to encompass (unless otherwise stated or clear from context) can refer in appropriate embodiments to any of the art-known or developed constructs or formats for capturing antibody structural and functional features in alternative presentation. For example, in some embodiments, the term can refer to bi- or other multi-specific (e.g., zybodies, etc.) antibodies, Small Modular ImmunoPharmaceuticals (“SMIPs™”), single chain antibodies, cameloid antibodies, and/or antibody fragments. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc]

“Associated”: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated entities are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Biocompatible”: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

“Biodegradable”: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

“Comparable”: The term “comparable”, as used herein, refers to two or more agents, entities, situations, sets of conditions, etc. that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

“Conjugated”: As used herein, the terms “conjugated,” “linked,” “attached,” and “associated with,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. Typically the moieties are attached either by one or more covalent bonds or by a mechanism that involves specific binding. Alternately, a sufficient number of weaker interactions can provide sufficient stability for moieties to remain physically associated.

“Corresponding to”: As used herein, the term “corresponding to” is often used to designate the position/identity of a residue in a polymer, such as an amino acid residue in a polypeptide or a nucleotide residue in a nucleic acid. Those of ordinary skill will appreciate that, for purposes of simplicity, residues in such a polymer are often designated using a canonical numbering system based on a reference related polymer, so that a residue in a first polymer “corresponding to” a residue at position 190 in the reference polymer, for example, need not actually be the 190th residue in the first polymer but rather corresponds to the residue found at the 190th position in the reference polymer; those of ordinary skill in the art readily appreciate how to identify “corresponding” amino acids, including through use of one or more commercially-available algorithms specifically designed for polymer sequence comparisons.

“Dosage form”: As used herein, the term “dosage form” refers to a physically discrete unit of a therapeutic agent for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

“Encapsulated”: The term “encapsulated” is used herein to refer to substances that are completely surrounded by another material.

“Functional”: As used herein, a “functional” biological molecule is a biological molecule in a form in which it exhibits a property and/or activity by which it is characterized. A biological molecule may have two functions (i.e., bi-functional) or many functions (i.e., multifunctional).

“Graft rejection”: The term “graft rejection” as used herein, refers to rejection of tissue transplanted from a donor individual to a recipient individual. In some embodiments, graft rejection refers to an allograft rejection, wherein the donor individual and recipient individual are of the same species. Typically, allograft rejection occurs when the donor tissue carries an alloantigen against which the recipient immune system mounts a rejection response.

“High Molecular Weight Polymer”: As used herein, the term “high molecular weight polymer” refers to polymers and/or polymer solutions comprised of polymers (e.g., protein polymers, such as silk) having molecular weights of at least about 200 kDa, and wherein no more than 30% of the silk fibroin has a molecular weight of less than 100 kDa. In some embodiments, high molecular weight polymers and/or polymer solutions have an average molecular weight of at least about 100 kDa or more, including, e.g., at least about 150 kDa, at least about 200 kDa, at least about 250 kDa, at least about 300 kDa, at least about 350 kDa or more. In some embodiments, high molecular weight polymers have a molecular weight distribution, no more than 50%, for example, including, no more than 40%, no more than 30%, no more than 20%, no more than 10%, of the silk fibroin can have a molecular weight of less than 150 kDa, or less than 125 kDa, or less than 100 kDa.

“Hydrolytically degradable”: As used herein, the term “hydrolytically degradable” is used to refer to materials that degrade by hydrolytic cleavage. In some embodiments, hydrolytically degradable materials degrade in water. In some embodiments, hydrolytically degradable materials degrade in water in the absence of any other agents or materials. In some embodiments, hydrolytically degradable materials degrade completely by hydrolytic cleavage, e.g., in water. By contrast, the term “non-hydrolytically degradable” typically refers to materials that do not fully degrade by hydrolytic cleavage and/or in the presence of water (e.g., in the sole presence of water).

“Hydrophilic”: As used herein, the term “hydrophilic” and/or “polar” refers to a tendency to mix with, or dissolve easily in, water.

“Hydrophobic”: As used herein, the term “hydrophobic” and/or “non-polar”, refers to a tendency to repel, not combine with, or an inability to dissolve easily in, water.

“Low Molecular Weight Polymer”: As used herein, the term “low molecular weight polymer” refers to polymers and/or polymer solutions, such as silk, comprised of polymers (e.g., protein polymers) having molecular weights within the range of about 20 kDa-about 400 kDa. In some embodiments, low molecular weight polymers (e.g., protein polymers) have molecular weights within a range between a lower bound (e.g., about 20 kDa, about 30 kDa, about 40 kDa, about 50 kDa, about 60 kDa, or more) and an upper bound (e.g., about 400 kDa, about 375 kDa, about 350 kDa, about 325 kDa, about 300 kDa, or less). In some embodiments, low molecular weight polymers (e.g., protein polymers such as silk) are substantially free of, polymers having a molecular weight above about 400 kD. In some embodiments, the highest molecular weight polymers in provided hydrogels are less than about 300-about 400 kD (e.g., less than about 400 kD, less than about 375 kD, less than about 350 kD, less than about 325 kD, less than about 300 kD, etc). In some embodiments, a low molecular weight polymer and/or polymer solution can comprise a population of polymer fragments having a range of molecular weights, characterized in that: no more than 15% of the total moles of polymer fragments in the population has a molecular weight exceeding 200 kDa, and at least 50% of the total moles of the silk fibroin fragments in the population has a molecular weight within a specified range, wherein the specified range is between about 3.5 kDa and about 120 kDa or between about 5 kDa and about 125 kDa.

“Nucleic acid”: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present disclosure. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, 4, 5, 6, 7, 8, 9, 10, or more residues. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present disclosure is specifically directed to “unmodified nucleic acids,” meaning nucleic acids (e.g., polynucleotides and residues, including nucleotides and/or nucleosides) that have not been chemically modified in order to facilitate or achieve delivery.

“Pharmaceutical composition”: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

“Physiological conditions”: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 6.8 to about 8.0 and a temperature range of about 20-40 degrees Celsius, about 25-40° C., about 30-40° C., about 35-40° C., about 37° C., atmospheric pressure of about 1. In some embodiments, physiological conditions utilize or include an aqueous environment (e.g., water, saline, Ringers solution, or other buffered solution); in some such embodiments, the aqueous environment is or comprises a phosphate buffered solution (e.g., phosphate-buffered saline).

“Polypeptide”: The term “polypeptide” as used herein, refers to a string of at least three amino acids linked together by peptide bonds. In some embodiments, a polypeptide comprises naturally-occurring amino acids; alternatively or additionally, in some embodiments, a polypeptide comprises one or more non-natural amino acids (i.e., compounds that do not occur in nature but that can be incorporated into a polypeptide chain; see, for example, http://www.cco.caltech.edu/~dadgrp/Unnatstruct.gif, which displays structures of non-natural amino acids that have been successfully incorporated into functional ion channels) and/or amino acid analogs as are known in the art may alternatively be employed). For example, a polypeptide can be a protein. In some embodiments, one or more of the amino acids in a polypeptide may be modified, for example, by the addition of a chemical entity such as a carbohydrate group, a phosphate group, a farnesyl group, an isofarnesyl group, a fatty acid group, a linker for conjugation, functionalization, or other modification, etc.

“Polysaccharide”: The term “polysaccharide” refers to a polymer of sugars. Typically, a polysaccharide comprises at least three sugars. In some embodiments, a polypeptide comprises natural sugars (e.g., glucose, fructose, galactose, mannose, arabinose, ribose, and xylose); alternatively or additionally, in some embodiments, a polypeptide comprises one or more non-natural amino acids (e.g. modified sugars such as 2′-fluororibose, 2′-deoxyribose, and hexose).

“Porosity”: The term “porosity” as used herein, refers to a measure of void spaces in a material and is a fraction of volume of voids over the total volume, as a percentage between 0 and 100%. A determination of a porosity is known to a skilled artisan using standardized techniques, for example mercury porosimetry and gas adsorption (e.g., nitrogen adsorption).

“Protein”: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

“Small molecule”: As used herein, the term “small molecule” is used to refer to molecules, whether naturally-occurring or artificially created (e.g., via chemical synthesis), having a relatively low molecular weight and being an organic and/or inorganic compound. Typically, a “small molecule” is monomeric and have a molecular weight of less than about 1500 g/mol. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is a modulating agent. In some embodiments, a small molecule is biologically active. In some embodiments, a small molecule is detectable (e.g., comprises at least one detectable moiety). In some embodiments, a small molecule is a therapeutic. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§ 330.5, 331 through 361, and 440 through 460; drugs for veterinary use listed by the FDA under 21 C.F.R. §§ 500 through 589, incorporated herein by reference, are all considered acceptable for use in accordance with the present application.

“Solution”: As used herein, the term “solution” broadly refers to a homogeneous mixture composed of one phase. Typically, a solution comprises a solute or solutes dissolved in a solvent or solvents. It is characterized in that the properties of the mixture (such as concentration, temperature, and density) can be uniformly distributed through the volume. In the context of the present application, therefore, a “silk fibroin solution” refers to silk fibroin protein in a soluble form, dissolved in a solvent, such as water. In some embodiments, silk fibroin solutions may be prepared from a solid-state silk fibroin material (i.e., silk matrices), such as silk films and other scaffolds. Typically, a solid-state silk fibroin material is reconstituted with an aqueous solution, such as water and a buffer, into a silk fibroin solution. It should be noted that liquid mixtures that are not homogeneous, e.g., colloids, suspensions, emulsions, are not considered solutions.

“Stable”: The term “stable,” when applied to compositions herein, means that the compositions maintain one or more aspects of their physical structure and/or activity over a period of time under a designated set of conditions. In some embodiments, the period of time is at least about one hour; in some embodiments, the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc. In some embodiments, the designated conditions are ambient conditions (e.g., at room temperature and ambient pressure). In some embodiments, the designated conditions are physiologic conditions (e.g., in vivo or at about 37° C. for example in serum or in phosphate buffered saline). In some embodiments, the designated conditions are under cold storage (e.g., at or below about 4° C., −20° C., or −70° C.). In some embodiments, the designated conditions are in the dark.

“Substantially”: As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Sustained release”: The term “sustained release” is used herein in accordance with its art-understood meaning of release that occurs over an extended period of time. The extended period of time can be at least about 3 days, about 5 days, about 7 days, about 10 days, about 15 days, about 30 days, about 1 month, about 2 months, about 3 months, about 6 months, or even about 1 year. In some embodiments, sustained release is substantially burst-free. In some embodiments, sustained release involves steady release over the extended period of time, so that the rate of release does not vary over the extended period of time more than about 5%, about 10%, about 15%, about 20%, about 30%, about 40% or about 50%. In some embodiments, sustained release involves release with first-order kinetics. In some embodiments, sustained release involves an initial burst, followed by a period of steady release. In some embodiments, sustained release does not involve an initial burst. In some embodiments, sustained release is substantially burst-free release.

“Therapeutic agent”: As used herein, the phrase “therapeutic agent” refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

“Treating”: As used herein, the term “treating” refers to partially or completely alleviating, ameliorating, relieving, inhibiting, preventing (for at least a period of time), delaying onset of, reducing severity of, reducing frequency of and/or reducing incidence of one or more symptoms or features of a particular disease, disorder, and/or condition. In some embodiments, treatment may be administered to a subject who does not exhibit symptoms, signs, or characteristics of a disease and/or exhibits only early symptoms, signs, and/or characteristics of the disease, for example for the purpose of decreasing the risk of developing pathology associated with the disease. In some embodiments, treatment may be administered after development of one or more symptoms, signs, and/or characteristics of the disease.

### DETAILED DESCRIPTION OF CERTAIN EMBODIMENTS

Among other things, the present disclosure provides stents. The present disclosure is directed to bioresorbable silk fibroin tracheal stents and methods and devices for deployment of the silk fibroin tracheal stents.

In some embodiments, stents can be fabricated according one of several embodiments that complement each other and provide for a broad range of stenting applications. In some embodiments, stents are arranged and constructed to be implanted as a tracheal stent.

In some embodiments, silk fibroin based stent grafts are externally affixed to an anterior tracheal wall. In some embodiments, a stent is designed to support a tracheal wall and prevent tracheal collapse.

In some embodiments, possible indications for a silk fibroin based tracheal stent include suprastomal collapse, tracheal stenosis, or tracheomalacia. In some embodiments, provide stents are particularly useful for pediatric patients.

In a recent study, bioresorbable plates have been employed to treat refractory localized airway malacia in patients undergoing corrective surgery for complex multilevel laryngotracheal stenosis. (See Gorostidi, F., et al., “External Bioresorbable Airway Rigidification to Treat Refractory Localized Tracheomalacia” 126 Laryngoscope, 2605 (2016). The study reported on seven patients (6 children, 1 adult). Subjects with a secondary malacic airway segments were diagnosed via by a dynamic transnasal flexible laryngotracheobronchoscopy before surgery. Extraluminal bioresorbable plates were used to stabilize the malacic segment through a transcervical approach under intraoperative flexible endoscopic guidance. External tracheal stabilization by stiffening using the plates allowed for complete or partial resolution of refractory proximal airway malacia in most cases.

Typically, stents provide an immediate mechanical support to open the lumen, which improves tracheal patency and prevents restenosis after implantation. However, the goals of stenting are achieved within weeks to months after implantation (see Waksman R, Biodegradable Stents: They Do their Job and Disappear: Why Bioabsorbable Stents?, J Invasive Cardiol., 2006, 18(2): 70-74). Recent research suggests that the response of the vessel wall to stent deployment reveals the role of the implant can be temporary because the mechanical stresses produced by stent implantation induces remodeling of the vessel walls (see Freeman et al., A link between stent radial forces and vascular wall remodeling: the discovery of an optimal stent radial force for minimal vessel restenosis, Connective Tissue Research, 2010, 51(4): 314-326). The continued presence of the stent becomes unnecessary and in some cases becomes deleterious. Current stent technology permanently remains in the vessel, which introduces many limitations including the risk of early and late thrombosis requiring the permanent use of P2Y₁₂ inhibitors for anti-platelet drug treatment (see Van Belle et al, Drug-eluting stents: trading restenosis for thrombosis?, J Thrombosis and Haemostasis, 2007, Suppl 1(January):238-245). Furthermore, current permanent stents generate additional concerns about late malapposition, hypersensitivity reactions, incomplete endothelialization or long-term impairment of endothelial response, elimination of vasomotion within the stented segment, and target lesion revascularization rates (see Gomes et al., Coronary stening and inflammation: implications for further surgical and medical treatment, Annals of Thoracic Surgery, 2006, 81(5): 1918-1925; see also Hofma et al., Increasing arterial wall injury after long-term implantation of two types of stent in a porcine coronary model, European Heart Journal, 1998, 19(4):601-609; Palmerini et al., Stent thrombosis with drug-eluting and bare-metal stents: evidence from a comprehensive network meta-analysis, Lancet, 2012, 379(9824):1393-1402).

The first resorbable stent implanted in humans, developed by Kyoto Medical Planning Company (Kyoto, Japan) was a balloon-mounted self-expanding design constructed from poly-L-lactic acid (PLLA), which degrades by bulk erosion (see Nishio et al., Long Term (>10 years) clinical outcomes of first-in-human biodegradable poly-1-lactic acid coronary stents, Circulation, 2012, 125(19):2343-2353). In the absorption process, hydrolysis of bonds between repeating lactide units produces lactic acid that enters the Krebs cycle and is metabolized to carbon dioxide and water. This device received a CE Mark in 2007 and is sold under the name REMEDY in Europe. The balloon-mounted deployment system requires expansion to be hastened by dilatation with contrast medium at a temperature of 80° C., which makes use cumbersome (see Nishio et al). Abbott Vascular (Santa Clara, Calif.) later developed the ABSORB polylactic acid everolimus-eluting stent producing clinical and imaging outcomes similar to those following metallic drug-eluting stents (see NIHR HSC, Bioresorbable stents for occlusive coronary artery disease, Birmingham: NIHR Horizon Scanning Centre (NIHR-HSC), Horizon Scanning Review, 2012). Although not available for sale in the United States, ABSORB received C E Mark in 2011. However, future development must target prevention of stent shrinkage exhibited by the ABSORB stent, after implantation in vivo (see Ormiston and Serruys, Bioabsorbable coronary stents, Circulation, 2009, 2(3):255-260).

Reva Medical Inc. (San Diego, Calif.) developed a resorbable stent using a tyrosine-derived polycarbonate polymer that metabolizes to amino acids, ethanol, and carbon dioxide (see Ormiston and Serruys). This is balloon expandable with a slide and lock (ratchet) design that allows stent expansion without material deformation. The REZORB first-in-man trial, which did not utilize a drug coating, had primary end points of major adverse events, such as, myocardial infarction, within 30 days (see Ormiston and Serruys). Further restenosis due to focal mechanical failures increased target lesion revascularization rate within 4 to 6 months (see Gonzalo and Macaya, Absorbable stent: focus on clinical applications and benefits, Vascular Health and Risk Management, 2012, 8:125-132). As a result, Reva is developing the ReZolve stent, a sirolimus-eluting revision with improved polymer strength.

The IDEAL stent, developed by Bioabsorbable Therapeutics Inc. (Menlo Park, Calif.) is a drug-eluting stent composed of poly(anhydride ester) salicylic acid (see Gonzalo and Macaya). The coating polymer is repeating salicylate molecules linked by adipic acid molecules while different linker molecules are used to join the stent backbone (see Ormiston and Serruys). This stent is designed to elute sirolimus but also releases salicylic acid as bonds are hydrolyzed during absorption. Absorption of the IDEAL stent, which is expected to be complete within 6 to 12 months, progresses by surface erosion (see Ormiston and Serruys). However, initial trials produced higher than expected intimal hyperplasia and restenosis necessitating design revisions (see Ormiston and Serruys). As a result, future revisions may include reducing strut thickness, percent wall coverage, and increasing sirolimus dosing.

Biotronik (Berlin, Germany) has made considerable advancements to balloon expandable magnesium alloy stents. These stents, which are laser cut from tubular magnesium WE-43 or AE21, generally exhibit better initial mechanical properties and radial strength compared to polymer variants (see Kwon D Y, Biodegradable stent, J Biomed Sci and Engineering, 2012, 05(04): 208-216). However, high rates of restenosis in the results of the PROGRESS AMS trial suggest loss of radial support during absorption happens prematurely (see Waksman et al., Early- and long-term intravascular ultrasound and angiographic findings after bioabsorbable magnesium stent implantation in human coronary arteries, JACC, 2009, 2(4): 312-320). Unlike polymer blends which undergo bulk erosion, absorption of magnesium stents occurs by surface erosion, which decreases strut thickness as the stent is absorbed (see Ormiston and Serruys). This may lead to an insufficient radial strength to counter the force of early remodeling (see Gonzalo and Macaya). Incomplete endothelialization which is characteristic of metallic degradable implants (see Ormiston and Serruys). Developments in this field are focused on perfecting control and tuning of degradation timing.

From the mechanical perspective, metallic stents which use deformation style deployment, rigidly maintain permanent diameters, and thus can potentially limit positive remodeling (see Ramcharitar and Serruys, Fully biodegradable coronary stents: progress to date, Amer J Cariovascular Drugs, 2008, 8(5): 305-314). Metallic stents which use self-expanding deployment fluctuate in diameter with vasomotion but do so by producing a shearing motion as struts slide past each other. The feature damages prior endothelialization and is a concern because damaged endothelial coverage is considered a main contributor to thrombosis following stent implantation (see Simons M, VEGF and restenosis: the rest of the story, Arteriosclerosis, thrombosis, and vascular boil., 29(4): 439-440).

Tracheomalacia is characterized by congenital or acquired deficiency of supporting tracheal cartilage and may result in airway collapse, respiratory distress, acute life-threatening events, or death. The estimated incidence of congenital tracheomalacia is 1 in 2100 newborn infants. (See Boogaard, R., et al., Tracheomalacia and bronchomalacia in children: incidence and patient characteristics, 128 Chest 5, 3391-7 (2005). While mild cases often resolve by age 24 months with conservative measures, more severe tracheomalacia necessitates intervention, sometimes including tracheostomy and ventilator support. Surgical intervention, when indicated, includes aortopexy, resection, tracheal stenting, or tracheoplasty. Tracheomalacia can also develop post-surgically following prolonged tracheostomy placement (suprastomal collapse) (20,000 such pediatric procedures have been performed over the past several decades in the US and Western Europe) (see http://emedicine.medscape.com/article/873805-overview#showall) or after tracheal surgery to treat tracheal stenosis or after removing tracheal tumors. A Cochrane review emphasizes that current available interventions are associated with high rates of failure and complications. (See Goyal, V., et al., Interventions for primary (intrinsic) tracheomalacia in children, 10 Cochrane Database Syst Rev, CD005304 (2012); see also Masters, I. B. and A. B. Chang, Interventions for primary (intrinsic) tracheomalacia in children, 4 Cochrane Database Syst Rev, CD005304 (2005).

Aortopexy relieves vascular compression on the trachea, and has a high success rate and low morbidity. (See Hoetzenecker K. et al., Pediatric airway surgery, 9 J Thorac Dis. 6, 663-1671 (2017)). However, the procedure has limited utility where long or multiple segments of the trachea are affected, or where the source of compression is not a nearby vessel. (See Deacon J W F, et al., Paediatric tracheomalacia—A review of clinical features and comparison of diagnostic imaging techniques, 98 Int J Pediatr Otorhinolaryngol, 75-81(2017)). Resection of the affected airway segment followed by anastomosis is another option to relieve symptoms of severe tracheomalacia. This technique is also limited to treating short segments of the airway, and there is a risk of tension on the anastomosis site. (See Ho A S et. al., Pediatric Tracheal Stenosis, 41 Otolaryngol Clin North Am. 5, 999-1021 (2008)). Internal stenting with silicone or metal stents offers a less invasive procedure and shorter recover time; however, formation of granulation tissue, stent migration, and difficult removal are common complications. (See Carden et al., Tracheomalacia and Tracheobronchomalacia in Children and Adults, 127 Chest, 3, 984-1005 (2005). A degradable and externally affixed splint aims to overcome these limitations associated with internal stents. (See Johnston et al., External Stent for Repair of Secondary Tracheomalacia, 30 Ann Thorac Surg. 3, 291-296 (1980)).

Prolonged tracheostomy can cause suprastomal collapse and/or granulation tissue formation. Suprastomal collapse occurs with an incidence of about 14-18% (see Benjamin, B., & Curley, J. W. Infant Tracheotomy—Endoscopy and Decannulation, 20 International Journal of Pediatric Otorhinolaryngology, 2, 113-121 (1990); see also Prescott, C., Peristomal Complications of Paediatric Tracheostomy, 23 International Journal of Pediatric Otorhinolaryngology, 2, 141-149 (1992)), making it a relatively common complication of pediatric tracheostomy. Treatment options in the event of suprastomal collapse or granulation tissue include endoscopic removal of granulation tissue and stenting with internal expandable stents, Aboulker stents (Teflon coated tube with tapered ends) or Montgomery T-tubes (silicone combination internal stent and tracheostomy tube). Internal stents can be a source of granuloma themselves, and pose the risk of migrating. Additionally, internal stents require eventual removal but can be impossible to remove endoscopically due to ingrowth and could require an additional invasive surgery. (See Ho, A. S., & Koltai, P. J., Pediatric Tracheal Stenosis, 41 Otolaryngologic Clinics of North America, 5, 999-1021 (2008)).

Tracheal stenosis is characterized by a narrowing of the tracheal lumen, making it difficult or impossible to breath. Stenosis can be either congenital or acquired in etiology. Congenital tracheal stenosis is relatively rare, occurring in an estimated 1 in 64,500 infants. (See (See Ho, A. S., & Koltai, P. J., Pediatric Tracheal Stenosis, 41 Otolaryngologic Clinics of North America, 5, 999-1021 (2008)). Acquired tracheal stenosis may result from prolonged intubation or tracheostomy, trauma, recurrent infections, or caustic aspiration, among other causes. Surgical treatment options include tracheal resection and reconstruction, and slide tracheoplasty. Both treatments effectively shorten the trachea, putting tension on the newly anastomosed tissue and increasing the risk of restenosis or leakage. (See Anton-Pacheco, J. L., Management of Congenital Tracheal Stenosis in Infancy. 29 European Journal of Cardio-Thoracic Surgery, 6, 991-996 (2006). A resorbable external tracheal stent to reinforce the reconstructed tissue and hold the trachea open radially while it heals would have the potential to greatly improve post-surgical morbidity and mortality.

External resorbable stents have been developed for tracheomalacia at University of Michigan and University of Wisconsin (Table 1). These stents are closer to 360 around, have relatively long degradation times of 3 years, and utilize synthetic polyesters polyglycolic acid (PGA), poly(lactide-co-glycolide) (PLGA), and polycaprolactone (PCL).

TABLE 1
Summary of resorbable external tracheal stent research

| Year | Institute | Materials | Fabrication | Degradation | Model | Outcome |
|---|---|---|---|---|---|---|
| 2000 | University | PGA:PLGA | PGA | 50% mass | Rabbit, | No advantage |
| | of | 85:15 | wrapped | lost at 13 | 3 months | over control; |
| | Michigan | | around a rod | weeks, 100% | | Material |
| | | | and dipped | at 20 weeks | | degrades too |
| | | | in PLGA | in vitro; | | quickly |
| | | | | slightly | | |
| | | | | quicker rate | | |
| | | | | in vivo | | |
| 2003 | University | PLGA | Flat PLGA | Not reported | Porcine, | No stenosis or |
| | of | | sheet heat- | | 4 months | respiratory |
| | Wisconsin | | shaped into a | | | issues at 4, 8, |
| | | | U | | | or 16 weeks |
| | | | conformation | | | |
| 2013 | University | PCL | 3D printed | Maintains | Porcine, | Significantly |
| | of | | (laser | support for | N = 3 | longer |
| | Michigan | | sintered); | 24 months, | control | survival in |
| | | | holes for | degrades | (no | stented |
| | | | suturing | fully by 3 | intervention), | group, (3.5-7 |
| | | | | years | N = 3 | days). |
| | | | | | stented | |
| 2013 | | | 3D printed | | Human | Off ventilator |
| | | | from patient | | patient, L | support after |
| | | | CT scan | | bronchus | 21 days |
| 2014 | | | | | Human | Still on low |
| | | | | | patient, | ventilator |
| | | | | | R and L | support ~8 |
| | | | | | bronchi, | weeks after |
| | | | | | 18 | surgery |
| | | | | | month | |
| | | | | | old | |

Recent work performed at the University of Michigan does not rely on or use a naturally derived polymer. Silk fibroin tracheal stents as provided herein afford a greater potential to incorporate cells or therapeutic molecules. Furthermore, such tracheal stents provide the ability to address previously untreated conditions, for example tracheomalacia. Advantageously, when compared with these prior tracheal stents, silk fibroin tracheal stents as disclosed herein provide the ability to tune degradation rate (vs PCL) and avoid inflammatory degradation products (vs PLGA).

Internal tracheal stents have been utilized to treat tracheal collapse (intrinsic tracheomalacia) due to either suprastomal collapse or tracheal collapse following prolonged tracheostomy tube placement or tracheal surgery or to treat severe tracheomalacia. Complications of such stents include granulation tissue development and airway obstruction, the development of further tracheal stenosis from rubbing of the stent in the inner lumen of the airway, inflammation, and scar formation. These complications often require admission to the pediatric intensive care unit and are eventually fatal in many cases.

The present disclosure, encompasses a recognition that commercially available tracheal stents are not specifically designed for pediatric patients. In some embodiments, dimensions, mechanical strength, and degradation profiles of proposed stents are designed specifically to meet pediatric needs.

In some embodiments, stents are or include silk fibroin. In accordance with some embodiments of the disclosure, all or portions of the stent can be formed from a biopolymer or biopolymer blend, for example, silk fibroin and blends.

In some embodiments, silk fibroin tracheal stents are composite materials that include silk fibroin and a plasticizer.

In some embodiments, provided silk fibroin tracheal stents are tubular in shape. In some embodiments, provided stents are concentric. In some embodiments, provided silk fibroin tracheal stents are tubular and range of about 120° to about 360°. In some embodiments, when a stent is characterized by a range of about 180° to about 360° such a stent provides ample radial support. In some embodiments, a preferable range would be about 140° to about 180°.

In some embodiments, silk fibroin tracheal stents are characterized by a range of about 180° to about 360° provides ample radial support without necessitating a more invasive surgery to extent the stent around the posterior trachea.

In some embodiments, provided tracheal stent graphs include a radial opening between about 0° and about 240°. In some embodiments, provided tracheal stent graphs are substantially cylindrical and include a radial opening between about 0° and about 240°.

In some embodiments, provided silk fibroin tracheal stents that are greater than 180° may require more posterior trachea access. In some embodiments, proposed stents that are greater than 180° may require a more invasive surgery. In some embodiments, for example, a 360 stent would require moving nerves out of the way.

In some embodiments, provided silk fibroin tracheal stents are solid grafts. In some embodiments, provided stents are patterned. In some embodiments, patterned stents include certain areas cut out to better visualize a healing resection. In some embodiments, areas cut out are laser cut.

In some embodiments, silk fibroin tracheal stents include areas cut out provide holes for suturing a stent in place. In some embodiments, holes for suturing a stent in place are not be included.

In some embodiments, silk fibroin tracheal stent materials, such as silk fibroin easily pass a suture.

In some embodiments, silk fibroin tracheal stent designs may include barbs on proximal and/or distal ends of provided stents to prevent migration.

In some embodiments, silk fibroin tracheal stents include reinforcement.

In some embodiments, silk fibroin tracheal stents include a plurality of layers that provide additional radial support. In some embodiments, at least one additional layer includes a layer made of silk fibroin. In some embodiments, an additional layer are made of stronger, denser silk formulation could be attached to the stent grafts to increase radial strength. In some embodiments, additional layers of stronger silk could be achieved by increasing protein concentration, eliminating plasticizers, or changing processing conditions to eliminate porosity. In some embodiments, additional layers are made of or include other materials, including other biopolymers, polymers, and/or metals.

In some embodiments, silk fibroin tracheal stents include struts that provide additional radial support. In some embodiments, struts are made of silk fibroin. In some embodiments, struts are made of stronger, denser silk formulation could be attached to the stent grafts to increase radial strength. The silk struts could be achieved by increasing protein concentration, eliminating plasticizers, or changing processing conditions to eliminate porosity. In some embodiments, struts are made of or include other materials, including other biopolymers, polymers, and/or metals.

In some embodiments, silk fibroin based tracheal stent grafts resorb into a subject's body over a specified period after anterior tracheal wall support is no longer needed.

In some embodiments, external silk fibroin based tracheal stent grafts are useful for upper tracheal suprastomal collapse from prolonged tracheostomy tube placement. In some embodiments, external tracheal stents are useful as an adjuvant at time of closure of tracheocutaneous fistula closure, to bolster the tracheal lumen externally and to help prevent air leak and crepitus. In some embodiments, external silk fibroin based tracheal stent grafts are useful to support anastomosis following tracheal resection and re-anastomosis or slide tracheoplasty.

In some embodiments, provided silk fibroin based tracheal stent grafts are externally affixed to a trachea. In some embodiments, an externally affixed stent is less likely to cause irritation, inflammation, and granulation tissue, and also less likely to migrate. In some embodiments, external suturing is more robust and a lower risk procedure.

Recently, resorbable external tracheal stents have been developed in the hopes of treating life threatening tracheomalacia. (See Zopf, D. A., et al., Treatment of severe porcine tracheomalacia with a 3-dimensionally printed, bioresorbable, external airway splint, 140 JAMA Otolaryngol Head Neck Surg 1, 66-71 (2014); see also Zopf, D. A., et al., Bioresorbable airway splint created with a three-dimensional printer, 368 N Engl J Med. 21,. 2043-5 (2013).

Prior stents designs for suprastomal collapse and tracheal stenosis are non-degradable and would require a second surgery for removal once the patient's airway has healed and remodeled sufficiently. In some embodiments, provided stents are degradable. In some embodiments, provided stents are fully resorbable. In some embodiments, provided stents do not require a second intervention to remove.

Prior stents designs for suprastomal collapse and tracheal stenosis degrade by bulk hydrolysis. In some embodiments, provided stents degrade via enzymatic surface erosion. In some embodiments, degradation via enzymatic surface erosion results in a more controlled degradation, longer retention of mechanical properties, and better predictability of changes in mechanical properties over time.

In some embodiments, silk fibroin based tracheal stent grafts include radiopaque markers or agents so that such stents can be visualized under x-ray. In some embodiments, radiopaque agents such as barium sulfate or tantalum could be dispersed in a silk matrix solution, or marker dots or bands could be placed at the proximal and distal ends of the stent.

In some embodiments, resorbable external silk fibroin based tracheal stent grafts are design and constructed to that produce little inflammation. In some embodiments, provided stents degrade in a predictable fashion and that could provide prolonged structural support for the healing trachea.

In some embodiments, external silk fibroin based tracheal stent grafts produce no tissue inflammatory response.

In some embodiments, external silk fibroin based tracheal stent grafts degrade to amino acids predictably over time by enzymes present in body cells.

In some embodiments, external silk fibroin based tracheal stent grafts in vivo for 3 months to 2 years with tunable target lifetimes.

In some embodiments, external silk fibroin based tracheal stent grafts are coated with drugs such as topical antibiotics and topical steroids.

In some embodiments, resorbable silk fibroin based tracheal stent grafts have dimensions that are adjustable to accommodate any size airway from infancy through adulthood in terms of accommodating internal tracheal diameters of 6 to 14 mm with lengths of 1-2 cm.

In some embodiments, resorbable silk fibroin based tracheal stent grafts can be sterilized via autoclaving.

In some embodiments, resorbable silk fibroin based tracheal stent grafts are shelf stable for years. In some embodiments, resorbable silk fibroin based tracheal stent grafts are shelf stable for at least 5 years.

In some embodiments, resorbable external silk fibroin based tracheal stent grafts are suturable to allow for fixation onto the external aspect of the trachea and to allow for both bolstering and plication upwards and outwards to promote the greatest tracheal diameter.

### Silks

Silk is a natural protein fiber produced in a specialized gland of certain organisms. Silk production in organisms is especially common in the Hymenoptera (bees, wasps, and ants), and is sometimes used in nest construction. Other types of arthropod also produce silk, most notably various arachnids such as spiders (e.g., spider silk). Silk fibers generated by insects and spiders represent the strongest natural fibers known and rival even synthetic high performance fibers.

Silk has been a highly desired and widely used textile since its first appearance in ancient China. (See Elisseeff, “The Silk Roads: Highways of Culture and Commerce,” Berghahn Books/UNESCO, New York (2000); see also Vainker, “Chinese Silk: A Cultural History,” Rutgers University Press, Piscataway, N.J. (2004)). Glossy and smooth, silk is favored by not only fashion designers but also tissue engineers because it is mechanically tough but degrades harmlessly inside the body, offering new opportunities as a highly robust and biocompatible material substrate. (See Altman et al., Biomaterials, 24: 401 (2003); see also Sashina et al., Russ. J. Appl. Chem., 79: 869 (2006)).

Silk is naturally produced by various species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis.

In general, silk for use in accordance with the present disclosure may be produced by any such organism, or may be prepared through an artificial process, for example, involving genetic engineering of cells or organisms to produce a silk protein and/or chemical synthesis. In some embodiments of the present disclosure, silk is produced by the silkworm, Bombyx mori. Silk fibroin produced by silkworms, such as Bombyx mori, is the most common and represents an earth-friendly, renewable resource.

As is known in the art, silks are modular in design, with large internal repeats flanked by shorter (˜100 amino acid) terminal domains (N and C termini). Naturally-occurring silks have high molecular weight (200 to 350 kDa or higher) with transcripts of 10,000 base pairs and higher and >3000 amino acids (reviewed in Omenatto and Kaplan (2010) Science 329: 528-531). The larger modular domains are interrupted with relatively short spacers with hydrophobic charge groups in the case of silkworm silk. N- and C-termini are involved in the assembly and processing of silks, including pH control of assembly. The N- and C-termini are highly conserved, in spite of their relatively small size compared with the internal modules. Table 2, below, provides an exemplary list of silk-producing species and silk proteins:

TABLE 2
An exemplary list of silk-producing species and silk proteins (adopted
from Bini et al. 335 J. Mol. Biol. 1, 27-40 (2003)).

| | | | |
|---|---|---|---|
| Accession | Species | Producing gland | Protein |

A. Silkworms

| | | | |
|---|---|---|---|
| AAN28165 | Antheraea mylitta | Salivary | Fibroin |
| AAC32606 | Antheraea pernyi | Salivary | Fibroin |
| AAK83145 | Antheraea yamamai | Salivary | Fibroin |
| AAG10393 | Galleria mellonella | Salivary | Heavy-chain fibroin |
| | | | (N-terminal) |
| AAG10394 | Galleria mellonella | Salivary | Heavy-chain fibroin |
| | | | (C-terminal) |
| P05790 | Bombyx mori | Salivary | Fibroin heavy chain |
| | | | precursor, Fib-H, H-fibroin |
| CAA27612 | Bombyx mandarina | Salivary | Fibroin |
| Q26427 | Galleria mellonella | Salivary | Fibroin light chain precursor, |
| | | | Fib-L, L-fibroin, PG-1 |
| P21828 | Bombyx mori | Salivary | Fibroin light chain precursor, |
| | | | Fib-L, L-fibroin |

B. Spiders

| | | | |
|---|---|---|---|
| P19837 | Nephila clavipes | Major ampullate | Spidroin 1, |
| | | | dragline silk fibroin 1 |
| P46804 | Nephila clavipes | Major ampullate | Spidroin 2, |
| | | | dragline silk fibroin 2 |
| AAK30609 | Nephila senegalensis | Major ampullate | Spidroin 2 |
| AAK30601 | Gasteracantha | Major ampullate | Spidroin 2 |
| | mammosa | | |
| AAK30592 | Argiope aurantia | Major ampullate | Spidroin 2 |
| AAC47011 | Araneus diadematus | Major ampullate | Fibroin-4, ADF-4 |
| AAK30604 | Latrodectus | Major ampullate | Spidroin 2 |
| | geometricus | | |
| AAC04503 | Araneus bicentenarius | Major ampullate | Spidroin 2 |
| AAK30615 | Tetragnatha versicolor | Major ampullate | Spidroin 1 |
| AAN85280 | Araneus ventricosus | Major ampullate | Dragline silk protein-1 |
| AAN85281 | Araneus ventricosus | Major ampullate | Dragline silk protein-2 |
| AAC14589 | Nephila clavipes | Minor ampullate | MiSp1 silk protein |
| AAK30598 | Dolomedes tenebrosus | Ampullate | Fibroin 1 |
| AAK30599 | Dolomedes tenebrosus | Ampullate | Fibroin 2 |
| AAK30600 | Euagrus chisoseus | Combined | Fibroin 1 |
| AAK30610 | Plectreurys tristis | Larger ampule- | Fibroin 1 |
| | | shaped | |
| AAK30611 | Plectreurys tristis | Larger ampule- | Fibroin 2 |
| | | shaped | |
| AAK30612 | Plectreurys tristis | Larger ampule- | Fibroin 3 |
| | | shaped | |
| AAK30613 | Plectreurys tristis | Larger ampule- | Fibroin 4 |
| | | shaped | |
| AAK30593 | Argiope trifasciata | Flagelliform | Silk protein |
| AAF36091 | Nephila | Flagelliform | Fibroin, silk protein |
| | madagascariensis | | (N-terminal) |
| AAF36092 | Nephila | Flagelliform | Silk protein |
| | madagascariensis | | (C-terminal) |
| AAC38846 | Nephila clavipes | Flagelliform | Fibroin, silk protein |
| | | | (N-terminal) |
| AAC38847 | Nephila clavipes | Flagelliform | Silk protein |
| | | | (C-terminal) |

### Silk Fibroin

Fibroin is a type of structural protein produced by certain spider and insect species that produce silk. Cocoon silk produced by the silkworm, Bombyx mori, is of particular interest because it offers low-cost, bulk-scale production suitable for a number of commercial applications, such as textile.

Silkworm cocoon silk contains two structural proteins, the fibroin heavy chain (˜350 kDa) and the fibroin light chain (˜25 kDa), which are associated with a family of non-structural proteins termed sericin, which glue the fibroin brings together in forming the cocoon. The heavy and light chains of fibroin are linked by a disulfide bond at the C-terminus of the two subunits. (See Takei, F., et al., 105 J. Cell Biol., 175-180 (1987); see also Tanaka, K., et al., 114 J. Biochem. (Tokyo), 1-4 (1993); Tanaka, K., et al., 1432 Biochim. Biophys. Acta., 92-103 (1999); Y Kikuchi, et al., “Structure of the Bombyx mori fibroin light-chain-encoding gene: upstream sequence elements common to the light and heavy chain,” 110 Gene, 151-158 (1992)). The sericins are a high molecular weight, soluble glycoprotein constituent of silk which gives the stickiness to the material. These glycoproteins are hydrophilic and can be easily removed from cocoons by boiling in water.

As used herein, the term “silk fibroin” refers to silk fibroin protein, whether produced by silkworm, spider, or other insect, or otherwise generated. (See Lucas et al., 13 Adv. Protein Chem., 107-242 (1958)). In some embodiments, silk fibroin is obtained from a solution containing a dissolved silkworm silk or spider silk. For example, in some embodiments, silkworm silk fibroins are obtained, from the cocoon of Bombyx mori. In some embodiments, spider silk fibroins are obtained, for example, from Nephila clavipes. In the alternative, in some embodiments, silk fibroins suitable for use in the disclosure are obtained from a solution containing a genetically engineered silk harvested from bacteria, yeast, mammalian cells, transgenic animals or transgenic plants. See, e.g., WO 97/08315 and U.S. Pat. No. 5,245,012, each of which is incorporated herein as reference in its entirety.

Thus, in some embodiments, a silk solution is used to fabricate compositions of the present disclosure contain fibroin proteins, essentially free of sericins. In some embodiments, silk solutions used to fabricate various compositions of the present disclosure contain the heavy chain of fibroin, but are essentially free of other proteins. In some embodiments, silk solutions used to fabricate various compositions of the present disclosure contain both the heavy and light chains of fibroin, but are essentially free of other proteins. In some embodiments, silk solutions used to fabricate various compositions of the present disclosure include both a heavy and a light chain of silk fibroin. In some embodiments, heavy chain and light chain of silk fibroin are linked via at least one disulfide bond. In some embodiments, where heavy and light chains of fibroin are present, they are linked via one, two, three or more disulfide bonds. Although different species of silk-producing organisms, and different types of silk, have different amino acid compositions, various fibroin proteins share certain structural features. A general trend in silk fibroin structure is a sequence of amino acids that is characterized by usually alternating glycine and alanine, or alanine alone. Such configuration allows fibroin molecules to self-assemble into a beta-sheet conformation. These “Alanine-rich” hydrophobic blocks are typically separated by segments of amino acids with bulky side-groups (e.g., hydrophilic spacers).

In some embodiments, polymers refers to peptide chains or polypeptides having an amino acid sequence corresponding to fragments derived from silk fibroin protein or variants thereof. In the context of stents of the present disclosure, silk fibroin fragments generally refer to silk fibroin peptide chains or polypeptides that are smaller than naturally occurring full length silk fibroin counterpart, such that one or more of the silk fibroin fragments within a population or composition. In some embodiments, for example, silk fibroin-based stents include silk fibroin polypeptides having an average molecular weight of between about 3.5 kDa and about 350 kDa. In some embodiments, suitable ranges of silk fibroin fragments include, but are not limited to: silk fibroin polypeptides having an average molecular weight of between about 3.5 kDa and about 200 kDa; silk fibroin polypeptides having an average molecular weight of between about 3.5 kDa and about 150 kDa; silk fibroin polypeptides having an average molecular weight of between about 3.5 kDa and about 120 kDa. In some embodiments, silk fibroin polypeptides have an average molecular weight of: about 3.5 kDa, about 4 kDa, about 4.5 kDa, about 5 kDa, about 6 kDa, about 7 kDa, about 8 kDa, about 9 kDa, about 10 kDa, about 15 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 105 kDa, about 110 kDa, about 115 kDa, about 120 kDa, about 125 kDa, about 150 kDa, about 200 kDa, about 250 kDa, about 300 kDa, or about 350 kDa. In some preferred embodiments, silk fibroin polypeptides have an average molecular weight of about 100 kDa.

In some embodiments, silk fibroin-based tracheal stents are or include silk fibroin and/or silk fibroin fragments. In some embodiments, silk fibroin and/or silk fibroin fragments of various molecular weights may be used. In some embodiments, silk fibroin and/or silk fibroin fragments of various molecular weights are silk fibroin polypeptides. In some embodiments, silk fibroin polypeptides are “reduced” in size, for instance, smaller than the original or wild type counterpart, may be referred to as “low molecular weight silk fibroin.” For more details related to low molecular weight silk fibroins, see WO 2014/145002, entitled “LOW MOLECULAR WEIGHT SILK AND STABILIZING SILK COMPOSITIONS,” the entire contents of which are incorporated herein by reference. In some embodiments, silk fibroin polypeptides have an average molecular weight of: less than 350 kDa, less than 300 kDa, less than 250 kDa, less than 200 kDa, less than 175 kDa, less than 150 kDa, less than 120 kDa, less than 100 kDa, less than 90 kDa, less than 80 kDa, less than 70 kDa, less than 60 kDa, less than 50 kDa, less than 40 kDa, less than 30 kDa, less than 25 kDa, less than 20 kDa, less than 15 kDa, less than 12 kDa, less than 10 kDa, less than 9 kDa, less than 8 kDa, less than 7 kDa, less than 6 kDa, less than 5 kDa, less than 4 kDa, less than 3.5 kDa, less than 3 kDa, less than 2.5 kDa, less than 2 kDa, less than 1.5 kDa, or less than about 1.0 kDa, etc.

In some embodiments, polymers of silk fibroin fragments can be derived by degumming silk cocoons at or close to (e.g., within 5% around) an atmospheric boiling temperature for at least about: 1 minute of boiling, 2 minutes of boiling, 3 minutes of boiling, 4 minutes of boiling, 5 minutes of boiling, 6 minutes of boiling, 7 minutes of boiling, 8 minutes of boiling, 9 minutes of boiling, 10 minutes of boiling, 11 minutes of boiling, 12 minutes of boiling, 13 minutes of boiling, 14 minutes of boiling, 15 minutes of boiling, 16 minutes of boiling, 17 minutes of boiling, 18 minutes of boiling, 19 minutes of boiling, 20 minutes of boiling, 25 minutes of boiling, 30 minutes of boiling, 35 minutes of boiling, 40 minutes of boiling, 45 minutes of boiling, 50 minutes of boiling, 55 minutes of boiling, 60 minutes or longer, including, e.g., at least 70 minutes, at least 80 minutes, at least 90 minutes, at least 100 minutes, at least 110 minutes, at least about 120 minutes or longer. As used herein, the term “atmospheric boiling temperature” refers to a temperature at which a liquid boils under atmospheric pressure.

In some embodiments, silk fibroin-based tracheal stents the present disclosure produced from silk fibroin fragments can be formed by degumming silk cocoons in an aqueous solution at temperatures of: about 30° C., about 35° C., about 40° C., about 45° C., about 50° C., about 45° C., about 60° C., about 65° C., about 70° C., about 75° C., about 80° C., about 85° C., about 90° C., about 95° C., about 100° C., about 105° C., about 110° C., about 115° C., about at least 120° C.

In some embodiments, such elevated temperature can be achieved by carrying out at least portion of the heating process (e.g., boiling process) under pressure. For example, suitable pressure under which silk fibroin fragments described herein can be produced are typically between about 10-40 psi, e.g., about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 31 psi, about 32 psi, about 33 psi, about 34 psi, about 35 psi, about 36 psi, about 37 psi, about 38 psi, about 39 psi, or about 40 psi.

In some embodiments, silk fibroin fragments solubilized. In some embodiments, a carrier can be a solvent or dispersing medium. In some embodiments, a solvent and/or dispersing medium, for example, is water, cell culture medium, buffers (e.g., phosphate buffered saline), a buffered solution (e.g. PBS), polyol (for example, glycerol, propylene glycol, liquid polyethylene glycol, and the like), Dulbecco's Modified Eagle Medium, fetal bovine serum, or suitable combinations and/or mixtures thereof.

In some embodiments, silk fibroin-based tracheal stents are modulated by controlling a silk concentration. In some embodiments, a weight percentage of silk fibroin can be present in the solution at any concentration suited to the need. In some embodiments, an aqueous silk fibroin solution can have silk fibroin at a concentration of about 0.1 mg/mL to about 20 mg/mL. In some embodiments, an aqueous silk fibroin solution can include silk fibroin at a concentration of about less than 1 mg/mL, about less than 1.5 mg/mL, about less than 2 mg/mL, about less than 2.5 mg/mL, about less than 3 mg/mL, about less than 3.5 mg/mL, about less than 4 mg/mL, about less than 4.5 mg/mL, about less than 5 mg/mL, about less than 5.5 mg/mL, about less than 6 mg/mL, about less than 6.5 mg/mL, about less than 7 mg/mL, about less than 7.5 mg/mL, about less than 8 mg/mL, about less than 8.5 mg/mL, about less than 9 mg/mL, about less than 9.5 mg/mL, about less than 10 mg/mL, about less than 11 mg/mL, about less than 12 mg/mL, about less than 13 mg/mL, about less than 14 mg/mL, about less than 15 mg/mL, about less than 16 mg/mL, about less than 17 mg/mL, about less than 18 mg/mL, about less than 19 mg/mL, or about less than 20 mg/mL.

Silk materials explicitly exemplified herein were typically prepared from material spun by silkworm, Bombyx mori. Typically, cocoons are boiled in an aqueous solution of 0.02 M Na₂CO₃, then rinsed thoroughly with water to extract the glue-like sericin proteins.

Extracted silk is then dissolved in a solvent, for example, LiBr (such as 9.3 M) solution at room temperature. Salts useful for this purpose include lithium bromide, lithium thiocyanate, calcium nitrate or other chemicals capable of solubilizing silk fibroin. In some embodiments, the extracted silk fibroin is dissolved in about 8M -12 M LiBr solution. The salt is consequently removed using, for example, dialysis. According to various embodiments, the boil time of B. mori cocoons may be varied in order to adjust the molecular weight of the silk fibroin material, for example, to alter the resorption characteristics and drug release profile of provided stents. A resulting silk fibroin solution can then be further processed for a variety of applications as described elsewhere herein.

If necessary, the solution can then be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, M W CO 3500) can be used. However, any dialysis system can be used. The dialysis can be performed for a time period sufficient to result in a final concentration of aqueous silk fibroin solution between about 10% to about 30%. In most cases dialysis for 2-12 hours can be sufficient. See, for example, International Patent Application Publication No. WO 2005/012606, the content of which is incorporated herein by reference in its entirety.

Alternatively, the silk fibroin solution can be produced using organic solvents. Such methods have been described, for example, in Li, M., et al., J. Appl. Poly Sci. 2001, 79, 2192-2199; Min, S., et al. Sen'I Gakkaishi 1997, 54, 85-92; Nazarov, R. et al., Biomacromolecules 2004 May-June; 5(3):718-26, content of all which is incorporated herein by reference in their entirety. An exemplary organic solvent that can be used to produce a silk fibroin solution includes, but is not limited to, hexafluoroisopropanol (HFIP). See, for example, International Application No. WO2004/000915, content of which is incorporated herein by reference in its entirety. Accordingly, in some embodiments, the solution comprising the silk fibroin includes an organic solvent, e.g., HFIP. In some other embodiments, the solution comprising the silk fibroin is free or essentially free of organic solvents.

Generally, any amount of silk fibroin can be present in the solution. For example, amount of silk fibroin in the solution can be from about 1% (w/v) to about 50% (w/v) of silk fibroin, e.g., silk fibroin. In some embodiments, the amount of silk fibroin in the solution can be from about 1% (w/v) to about 35% (w/v), from about 1% (w/v) to about 30% (w/v), from about 1% (w/v) to about 25% (w/v), from about 1% (w/v) to about 20% (w/v), from about 1% (w/v) to about 15% (w/v), from about 1% (w/v) to about 10% (w/v), from about 5% (w/v) to about 25% (w/v), from about 5% (w/v) to about 20% (w/v), from about 5% (w/v) to about 15% (w/v). In some embodiments, the amount of silk fibroin in the solution is less than 5% (w/v). In some embodiments, the amount of silk fibroin in the solution is greater than 25% (w/v). Exact amount of silk fibroin in the silk fibroin solution can be determined by drying a known amount of the silk fibroin solution and measuring the mass of the residue to calculate the solution concentration.

In some embodiments, an amount of silk fibroin in solution is for example, about 10% (w/w) to about 50% (w/w) or about 20% (w/w) to about 40% (w/w). In some embodiments, the amount of silk fibroin in the solution is about 5% (w/w), about 10% (w/w), about 15% (w/w), about 20% (w/w), about 25% (w/w), about 30% (w/w), about 35% (w/w), about 40% (w/w), about 45% (w/w), or about 50% (w/w).

In some embodiments, silk fibroin-based tracheal stents form a porous matrix or scaffold. For example, the porous scaffold can have a porosity of at least about 10%, at least about 20%, at least about 30%, at least about 40%, at least about 50%, at least about 60%, at least about 70%, at least about 80%, at least about 90%, or higher.

### Degradation Properties of Silk-Based Materials

Additionally, as will be appreciated by those of skill in the art, much work has established that researchers have the ability to control the degradation process of silk. According to the present disclosure, such control can be particularly valuable in the fabrication of electronic components, and particularly of electronic components that are themselves and/or are compatible with biomaterials. Degradability (e.g., bio-degradability) is often essential for biomaterials used in tissue engineering and implantation. The present disclosure encompasses the recognition that such degradability is also relevant to and useful in the fabrication of silk electronic components.

According to the present disclosure, one particularly desirable feature of silk-based materials is the fact that they can be programmably degradable. That is, as is known in the art, depending on how a particular silk-based material is prepared, it can be controlled to degrade at certain rates. Degradability and controlled release of a substance from silk-based materials have been published (see, for example, WO 2004/080346, WO 2005/012606, WO 2005/123114, WO 2007/016524, WO 2008/150861, WO 2008/118133, each of which is incorporated by reference herein).

Control of silk material production methods as well as various forms of silk-based materials can generate silk compositions with known degradation properties. For example, using various silk fibroin materials (e.g., microspheres of approximately 2 μm in diameter, silk film, silk stents) entrapped agents such as therapeutics can be loaded in active form, which is then released in a controlled fashion, e.g., over the course of minutes, hours, days, weeks to months. It has been shown that layered silk fibroin coatings can be used to coat substrates of any material, shape and size, which then can be used to entrap molecules for controlled release, e.g., 2-90 days.

### Crystalline Silk Materials

As known in the art and as described herein, silk proteins can stack with one another in crystalline arrays. Various properties of such arrays are determined, for example, by the degree of beta-sheet structure in the material, the degree of cross-linking between such beta sheets, the presence (or absence) of certain dopants or other materials. In some embodiments, one or more of these features is intentionally controlled or engineered to achieve particular characteristics of a silk matrix. In some embodiments, silk fibroin-based stents are characterized by crystalline structure, for example, comprising beta sheet structure and/or hydrogen bonding. In some embodiments, provided silk fibroin-based stents are characterized by a percent beta sheet structure within the range of about 0% to about 45%. In some embodiments, silk fibroin-based stents are characterized by crystalline structure, for example, comprising beta sheet structure of about 1%, about 2%, about 3%, about 4%, about 5%, about 6%, about 7%, about 8%, about 9%, about 10%, about 11%, about 12%, about 13%, about 1%, about 1%, about 1%, about 1%, about 1%, about 1%, about 1%, about 1%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45%.

### Nanosized Crystalline Particles

In some embodiments, silk fibroin-based tracheal stents are characterized in that they include submicron size or nanosized crystallized spheres and/or particles. In some embodiments, such submicron size or nanosized crystallized spheres and/or particles have average diameters ranging between about 5 nm and 200 nm. In some embodiments, submicron size or nanosized crystallized spheres and/or particles have less than 150 nm average diameter, e.g., less than 145 nm, less than 140 nm, less than 135 nm, less than 130 nm, less than 125 nm, less than 120 nm, less than 115 nm, less than 110 nm, less than 100 nm, less than 90 nm, less than 80 nm, less than 70 nm, less than 60 nm, less than 50 nm, less than 40 nm, less than 30 nm, less than 20 nm, less than 15 nm, less than 10 nm, less than 5 nm, or smaller. In some preferred embodiments, submicron size or nanosized crystallized spheres and/or particles have average diameters of less than 100 nm.

Additives, Agents, and/or Functional Moieties

In some embodiments, a bulk material of a stent includes one or more (e.g., one, two, three, four, five or more) additives, agents, and/or functional moieties. Without wishing to be bound by a theory, additives, agents, and/or functional moieties can provide one or more desirable properties to the stent, e.g., strength, flexibility, ease of processing and handling, biocompatibility, bioresorability, lack of air bubbles, surface morphology, and the like. In some embodiments, additives, agents, and/or functional moieties can be covalently or non-covalently linked with silk fibroin and can be integrated homogenously or heterogeneously within the bulk material. In some embodiments, the active agent is absorbed/adsorbed on a surface of the stent.

In some embodiments, additives, agents, and/or functional moieties can be in any physical form. For example, additives, agents, and/or functional moieties can be in the form of a particle (e.g., microparticle or nanoparticle), a fiber, a film, a gel, a mesh, a mat, a non-woven mat, a powder, a liquid, or any combinations thereof. In some embodiments, a silk fibroin tracheal stent comprising additives, agents, and/or functional moieties can be formulated by mixing one or more additives, agents, and/or functional moieties with a silk fibroin-fibroin solution used to make such a stent.

In some embodiments, an additives, agents, and/or functional moieties are covalently associated (e.g., via chemical modification or genetic engineering). In some embodiments, additives, agents, and/or functional moieties are non-covalently associated.

Without limitations, additives, agents, and/or functional moieties can be selected from the group consisting of anti-proliferative agents, biopolymers, nanoparticles (e.g., gold nanoparticles), proteins, peptides, nucleic acids (e.g., DNA, RNA, siRNA, modRNA), nucleic acid analogs, nucleotides, oligonucleotides, peptide nucleic acids (PNA), aptamers, antibodies or fragments or portions thereof (e.g., paratopes or complementarity-determining regions), antigens or epitopes, hormones, hormone antagonists, growth factors or recombinant growth factors and fragments and variants thereof, cell attachment mediators (such as RGD), cytokines, enzymes, small molecules, antibiotics or antimicrobial compounds, toxins, therapeutic agents and prodrugs, small molecules and any combinations thereof.

In some embodiments, an additive, agent, or functional moiety is a polymer. In some embodiments, a polymer is a biocompatible polymer. As used herein, “biocompatible polymer” refers to any polymeric material that does not deteriorate appreciably and does not induce a significant immune response or deleterious tissue reaction, e.g., toxic reaction or significant irritation, over time when implanted into or placed adjacent to the biological tissue of a subject, or induce blood clotting or coagulation when it comes in contact with blood. Exemplary biocompatible polymers include, but are not limited to, a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, fibronectin, keratin, polyaspartic acid, alginate, chitosan, chitin, hyaluronic acid, pectin, polylactic acid, polyglycolic acid, polyhydroxyalkanoates, dextrans, and polyanhydrides, polyethylene oxide (PEO), poly(ethylene glycol) (PEG), triblock copolymers, polylysine, alginate, polyaspartic acid, any derivatives thereof and any combinations thereof. Other exemplary biocompatible polymers amenable to use according to the present disclosure include those described for example in U.S. Pat. Nos. 6,302,848; 6,395,734; 6,127,143; 5,263,992; 6,379,690; 5,015,476; 4,806,355; 6,372,244; 6,310,188; 5,093,489; 387,413; 6,325,810; 6,337,198; 6,267,776; 5,576,881; 6,245,537; 5,902,800; and 5,270,419, content of all of which is incorporated herein by reference.

In some embodiments, a biocompatible polymer is PEG or PEO. As used herein, term “polyethylene glycol” or “PEG” means an ethylene glycol polymer that contains about 20 to about 2000000 linked monomers, typically about 50-1000 linked monomers, usually about 100-300. PEG is also known as polyethylene oxide (PEO) or polyoxyethylene (POE), depending on its molecular weight. Generally PEG, PEO, and POE are chemically synonymous, but historically PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g. viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete PEGs are also available with different geometries.

As used herein, PEG is intended to be inclusive and not exclusive. In some embodiments, PEG includes poly(ethylene glycol) in any of its forms, including alkoxy PEG, difunctional PEG, multiarmed PEG, forked PEG, branched PEG, pendent PEG (i.e., PEG or related polymers having one or more functional groups pendent to the polymer backbone), or PEG With degradable linkages therein. Further, a PEG backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(-PEG-OH)m in which R represents the core moiety, such as glycerol or pentaerythritol, and m represents the number of arms. Multi-armed PEG molecules, such as those described in U.S. Pat. No. 5,932,462, which is incorporated by reference herein in its entirety, can also be used as biocompatible polymers.

Some exemplary PEGs include, but are not limited to, PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG15000, PEG 20000, PEG250000, PEG500000, PEG100000, PEG2000000 and the like. In some embodiments, PEG is of MW 10,000 Dalton. In some embodiments, PEG is of MW 100,000, i.e. PEO of MW 100,000.

In some embodiments, a polymer is a biodegradable polymer. As used herein, “biodegradable” describes a material which can decompose under physiological conditions into breakdown products. Such physiological conditions include, for example, hydrolysis (decomposition via hydrolytic cleavage), enzymatic catalysis (enzymatic degradation), and mechanical interactions. As used herein, “biodegradable” also encompasses “bioresorbable”, which describes a substance that decomposes under physiological conditions to break down to products that undergo bioresorption into the host-organism, namely, become metabolites of the biochemical systems of the host organism.

As used herein, “bioresorbable” and “bioresorption” encompass processes such as cell-mediated degradation, enzymatic degradation and/or hydrolytic degradation of the bioresorbable polymer, and/or elimination of the bioresorbable polymer from living tissue as will be appreciated by the person skilled in the art.

“Biodegradable polymer”, as used herein, refers to a polymer that at least a portion thereof decomposes under physiological conditions. A polymer can thus be partially decomposed or fully decomposed under physiological conditions.

Exemplary biodegradable polymers include, but are not limited to, polyanhydrides, polyhydroxybutyric acid, polyorthoesters, polysiloxanes, polycaprolactone, poly(lactic-co-glycolic acid), poly(lactic acid), poly(glycolic acid), and copolymers prepared from the monomers of these polymers.

In some embodiments, additives, agents, or functional moieties include a bioinert material. As used herein, a “bioinert” material refers to any material that once placed in vivo has minimal interaction with its surrounding tissue. Exemplary bioinert materials include, but are not limited to, gold, stainless steel, titanium, alumina, partially stabilized zirconia, and ultra-high molecular weight polyethylene.

In some embodiments, additives, agents, or functional moieties can be a silk fibroin particle or powder. Various methods of producing silk fibroin particles (e.g., nanoparticles and microparticles) are known in the art. See for example, PCT Publication No. WO 2011/041395 and No. WO 2008/118133; U.S. App. Pub. No. U.S. 2010/0028451; US Provisional Application Ser. No. 61/719,146, filed Oct. 26, 2012; and Wenk et al. J Control Release, Silk fibroin spheres as a platform for controlled drug delivery, 2008; 132: 26-34, content of all of which is incorporated herein by reference in their entirety.

In some embodiments, additives, agents, or functional moieties include silk fibroin fiber. In some embodiments, silk fibroin fibers could be chemically attached by redissolving part of the fiber in HFIP and attaching to stent. Use of silk fibroin fibers is described in, for example, US patent application publication no. US20110046686, content of which is incorporated herein by reference.

In some embodiments, silk fibroin fibers are microfibers or nanofibers. In some embodiments, additives, agents, or functional moieties are micron-sized silk fibroin fiber (10-600 p.m). Micron-sized silk fibroin fibers can be obtained by hydrolyzing degummed silk fibroin or by increasing a boiling time of a degumming process. Alkali hydrolysis of silk fibroin to obtain micron-sized silk fibroin fibers is described for example in Mandal et al., PNAS, 2012, doi: 10.1073/pnas.1119474109; U.S. Provisional Application No. 61/621,209, filed Apr. 6, 2012; and PCT application no. PCT/US13/35389, filed Apr. 5, 2013, content of all of which is incorporated herein by reference. Because regenerated silk fibroin fibers made from HFIP silk fibroin solutions are mechanically strong. the regenerated silk fibroin fibers can also be used as additive.

In some embodiments, silk fibroin fiber is an unprocessed silk fibroin fiber, e.g., raw silk fibroin or raw silk fibroin fiber. “Raw silk fibroin” or “raw silk fibroin fiber” refers to silk fibroin fiber that has not been treated to remove sericin, and thus encompasses, for example, silk fibroin fibers taken directly from a cocoon. Thus, by unprocessed silk fibroin fiber is meant silk fibroin, obtained directly from the silk fibroin gland. When silk fibroin, obtained directly from the silk fibroin gland, is allowed to dry, the structure is referred to as silk fibroin I in the solid state. Thus, an unprocessed silk fibroin fiber includes silk fibroin mostly in the silk fibroin I conformation. A regenerated or processed silk fibroin fiber on the other hand includes silk fibroin having a substantial silk fibroin II or beta-sheet crystallinity.

In some embodiments, a conformation of the fibroin in a stent can be altered before, during or after its formation. Induced conformational change alters silk fibroin crystallinity, e.g., Silk fibroin II beta-sheet crystallinity. Without wishing to be bound by a theory, it is believed that degradation of the bulk material or optional release of an additive (e.g., an active agent) from the bulk material varies with the beta-sheet content of the silk fibroin. Conformational change can be induced by any methods known in the art, including, but not limited to, alcohol immersion (e.g., ethanol, methanol), water annealing, shear stress (e.g., by vortexing), ultrasound (e.g., by sonication), pH reduction (e.g., pH titration and/or exposure to an electric field) and any combinations thereof. For example, a conformational change can be induced by one or more methods, including but not limited to, controlled slow drying (Lu et al., 10 Biomacromolecules 1032 (2009)); water annealing (Jin et al., Water-Stable Silk fibroin Films with Reduced β-Sheet Content, 15 Adv. Funct. Mats. 1241 (2005); Hu et al. Regulation of Silk fibroin Material Structure by Temperature-Controlled Water Vapor Annealing, 12 Biomacromolecules 1686 (2011)); stretching (Demura & Asakura, Immobilization of glucose oxidase with Bombyx mori silk fibroin by only stretching treatment and its application to glucose sensor, 33 Biotech & Bioengin. 598 (1989)); compressing; solvent immersion, including methanol (Hofmann et al., Silk fibroin as an organic polymer for controlled drug delivery, 111 J Control Release. 219 (2006)), ethanol (Miyairi et al., Properties of b-glucosidase immobilized in sericin membrane. 56 J. Fermen. Tech. 303 (1978)), glutaraldehyde (Acharya et al., Performance evaluation of a silk fibroin protein-based matrix for the enzymatic conversion of tyrosine to L-DOPA. 3 Biotechnol J. 226 (2008)), and 1-ethyl-3-(3-dimethyl aminopropyl) carbodiimide (EDC) (Bayraktar et al., Silk fibroin as a novel coating material for controlled release of theophylline. 60 Eur J Pharm Biopharm. 373 (2005)); pH adjustment, e.g., pH titration and/or exposure to an electric field (see, e.g., U.S. Patent App. No. US2011/0171239); heat treatment; shear stress (see, e.g., International App. No.: WO 2011/005381), ultrasound, e.g., sonication (see, e.g., U.S. Patent Application Publication No. U.S. 2010/0178304 and International App. No. WO2008/150861); and any combinations thereof. Content of all of the references listed above is incorporated herein by reference in their entirety.

In some embodiments, an additive, agent, and/or functional moiety is a plasticizer. As used herein, a “plasticizer” is intended to designate a compound or a mixture of compounds that can increase flexibility, processability and extensibility of the polymer in which it is incorporated. In some embodiments, a plasticizer can reduce the viscosity of the melt, lower the second order transition temperatures and the elastic modulus of the product. In some embodiments, suitable plasticizers include, but are not limited to, low molecular weight polyols having aliphatic hydroxyls such as ethylene glycol; propylene glycol; propanetriol (i.e., glycerol); glyceryl monostearate; 1,2-butylene glycol; 2,3-butylene glycol; styrene glycol; polyethylene glycols such as diethylene glycol, triethylene glycol, tetraethylene glycol and other polyethylene glycols having a molecular weight of about 1,000 or less; polypropylene glycols of molecular weight 200 or less; glycol ethers such as monopropylene glycol monoisopropyl ether; propylene glycol monoethyl ether; ethylene glycol monoethyl ether; diethylene glycol monoethyl ether; ester-type plasticizers such as sorbitol lactate, ethyl lactate, butyl lactate, ethyl glycolate, allyl glycolate; and amines such as monoethanolamine, diethanolamine, triethanolamine, monisopropanolamine, triethylenetetramine, 2-amino-2-methyl-1,3-propanediol, polymers and the like. In one embodiment, the plasticizer can include glycerol.

In some embodiments, plasticizers may be included in a silk formulation to augment properties or add new functionality. In some embodiments, an addition of 1-50% glycerol increased elasticity and compliance of such a stent. Specifically, a glycerol concentration of 5-10% by weight is most advantageous mechanical properties for this application. Lower concentrations of glycerol do no result in a detectable increase in elasticity, while higher concentrations compromise the stiffness of the stents. In some embodiments, glycerol is diluted with deionized water before being added to the silk solution. In some embodiments, glycerol solution concentrations of 350 mg/mL or lower, may induce gelation when added to silk. In some embodiments, such concentrations makes it nearly impossible to homogenize a solution, and to add in an accurate amount of glycerol. In some embodiments, a glycerol solution concentration of 700 mg/mL is preferred. In some embodiments, once added, a silk/glycerol solution is mixed by gentle inversion, aggressive sonication or vortex mixing can cause preemptive gelation.

In some embodimnts, provided silk fibroin tracheal stents include additives, agents, and/or functional moieties, for example, therapeutic, preventative, and/or diagnostic agents.

In some embodiments, a therapeutic agent can be selected from the group consisting of anti-infectives, chemotherapeutic agents, anti-rejection agents, analgesics and analgesic combinations, anti-inflammatory agents, hormones, growth factors, antibiotics, antiviral agents, steroids, bone morphogenic proteins, bone morphogenic-like proteins, epidermal growth factor, fibroblast growth factor, platelet derived growth factor (PDGF), insulin-like growth factor, transforming growth factors, vascular endothelial growth factor, and any combinations thereof.

In some embodiments, an additive is or includes one or more therapeutic agents. In general, a therapeutic agent is or includes a small molecule and/or organic compound with pharmaceutical activity (e.g., activity that has been demonstrated with statistical significance in one or more relevant pre-clinical models or clinical settings). In some embodiments, a therapeutic agent is a clinically-used drug. In some embodiments, a therapeutic agent is or includes an cells, proteins, peptides, nucleic acid analogues, nucleotides, oligonucleotides, nucleic acids (DNA, RNA, siRNA), peptide nucleic acids, aptamers, antibodies or fragments or portions thereof, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, hormones, hormone antagonists, growth factors or recombinant growth factors and fragments and variants thereof, cytokines, enzymes, antibiotics or antimicrobial compounds, antifungals, antivirals, toxins, prodrugs, chemotherapeutic agents, small molecules, drugs (e.g., drugs, dyes, amino acids, vitamins, antioxidants), pharmacologic agents, and combinations thereof.

In some embodiments, an additive, agent, and/or functional moiety is a therapeutic agent. A “therapeutic agent” refers to a biological or chemical agent used for treating, curing, mitigating, or preventing deleterious conditions in a subject. “Therapeutic agent” also includes substances and agents for combating a disease, condition, or disorder of a subject, and includes drugs, diagnostics, and instrumentation. “Therapeutic agent” also includes anything used in medical diagnosis, or in restoring, correcting, or modifying physiological functions. “Therapeutic agent” and “pharmaceutically active agent” are used interchangeably herein.

A therapeutic agent is selected according to the treatment objective and biological action desired. General classes of therapeutic agents include anti-microbial agents such as adrenergic agents, antibiotic agents or antibacterial agents, antiviral agents, anthelmintic agents, anti-inflammatory agents, antineoplastic agents, antioxidant agents, biological reaction inhibitors, botulinum toxin agents, chemotherapy agents, contrast imaging agents, diagnostic agents, gene therapy agents, hormonal agents, mucolytic agents, radioprotective agents, radioactive agents including brachytherapy materials, tissue growth inhibitors, tissue growth enhancers, and vasoactive agents. Therapeutic agent can be selected from any class suitable for the therapeutic objective. In some embodiments, a therapeutic agent is an antithrombotic or fibrinolytic agent selected from the group consisting of anticoagulants, anticoagulant antagonists, antiplatelet agents, thrombolytic agents, thrombolytic agent antagonists, and any combinations thereof.

In some embodiments, a therapeutic agent is thrombogenic agent selected from the group consisting of thrombolytic agent antagonists, anticoagulant antagonists, pro-coagulant enzymes, pro-coagulant proteins, and any combinations thereof. Some exemplary thrombogenic agents include, but are not limited to, protamines, vitamin K1, amiocaproic acid (amicar), tranexamic acid (amstat), anagrelide, argatroban, cilstazol, daltroban, defibrotide, enoxaparin, fraxiparine, indobufen, lamoparan, ozagrel, picotamide, plafibride, tedelparin, ticlopidine, triflusal, collagen, and collagen-coated particles.

In some embodiments, a therapeutic agent is a vasodilator. A vasodilator can be selected from the group consisting of alpha-adrenoceptor antagonists (alpha-blockers), agiotensin converting enzyme (ACE) inhibitors, angiotensin receptor blockers (ARBs), beta2-adrenoceptor agonists (β2-agonists), calcium-channel blockers (CCBs), centrally acting sympatholytics, direct acting vasodilators, endothelin receptor antagonists, ganglionic blockers, nitrodilators, phosphodiesterase inhibitors, potassium-channel openers, renin inhibitors, and any combinations thereof. Exemplary vasodilator include, but are not limited to, prazosin, terazosin, doxazosin, trimazosin, phentolamine, phenoxybenzamine, benazepril, captopril, enalapril, fosinopril, lisinopril, moexipril, quinapril, ramipril, candesartan, eprosartan, irbesartan, losartan, olmesartan, telmisartan, valsartan, Epinephrine, Norepinephrine, Dopamine, Dobutamine, Isoproterenol, amlodipine, felodipine, isradipine, nicardipine, nifedipine, nimodipine, nitrendipine, clonidine, guanabenz, guanfacine, a-methyldopa, hydralazine, Bosentan, trimethaphan camsylate, isosorbide dinitrate, isosorbide mononitrate, nitroglycerin, erythrityl tetranitrate, pentaerythritol tetranitrate, sodium nitroprusside, milrinone, inamrinone (formerly amrinone), cilostazol, sildenafil, tadalafil, minoxidil, aliskiren, nitric oxide, sodium nitrite, nitroglycerin, and analogs, derivatives, prodrugs, and pharmaceutically acceptable salts thereof.

Exemplary pharmaceutically active compound include, but are not limited to, those found in Harrison's Principles of Internal Medicine , 13th Edition, Eds. T. R. Harrison et al. McGraw-Hill N.Y., N.Y.; Physicians' Desk Reference, 50th Edition, 1997, Oradell N.J., Medical Economics Co.; Pharmacological Basis of Therapeutics, 8th Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety.

In some embodiments, active agents can be selected from small organic or inorganic molecules; saccharines; oligosaccharides; polysaccharides; biological macromolecules; peptides; proteins; peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof. The active agent can be hydrophobic, hydrophilic, or amphiphilic.

Small molecules can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some cases it is highly preferred that a small molecule have a molecular mass equal to or less than 700 Daltons.

In some embodiments, possible additives, agents, or functional moieties are soluble drugs that could be released into a local environment as the stent degrades, growth factors to stimulate local tissue regeneration, cell adhesion proteins to promote cellular infiltration, cleavable crosslinkers to further control degradation, or patient derived cells.

In some embodiments, a stent includes a biologically active agent. As used herein, “biological activity” or “bioactivity” refers to the ability of a molecule or composition to affect a biological sample. Biological activity can include, without limitation, elicitation of a stimulatory, inhibitory, regulatory, toxic or lethal response in a biological assay. For example, a biological activity can refer to the ability of a compound to modulate the effect/activity of an enzyme, block a receptor, stimulate a receptor, modulate the expression level of one or more genes, modulate cell proliferation, modulate cell division, modulate cell morphology, or any combination thereof. In some instances, a biological activity can refer to the ability of a compound to produce a toxic effect in a biological sample. A stent including an active agent can be formulated by mixing one or more active agents with the silk fibroin-fibroin solution used to make the stent.

Examples of biologically active compounds include, but are not limited to: cell attachment mediators, such as collagen, elastin, fibronectin, vitronectin, laminin, proteoglycans, or peptides containing known integrin binding domains e.g. “RGD” integrin binding sequence, or variations thereof, that are known to affect cellular attachment (Schaffner P & Dard, Cell Mol Life Sci,. 2003, 60(1):119-32 and Hersel U. et al., Biomaterials, 2003, 24(24):4385-415); YIGSR peptides; biologically active ligands; and substances that enhance or exclude particular varieties of cellular or tissue ingrowth.

In some embodiments, an active agent is an anti-restenosis or restenosis inhibiting agent. Suitable anti-restenosis agents include: (1) antiplatelet agents including: (a) thrombin inhibitors and receptor antagonists, (b) adenosine disphosphate (ADP) receptor antagonists (also known as purinoceptorᵢ receptor antagonists), (c) thromboxane inhibitors and receptor antagonists and (d) platelet membrane glycoprotein receptor antagonists; (2) inhibitors of cell adhesion molecules, including (a) selectin inhibitors and (b) integrin inhibitors; (3) anti-chemotactic agents; (4) interleukin receptor antagonists (which also serve as anti-pain/anti-inflammation agents); and (5) intracellular signaling inhibitors including: (a) protein kinase C (PKC) inhibitors and protein tyrosine kinase inhibitors, (b) modulators of intracellular protein tyrosine phosphatases, (c) inhibitors of src homology₂ (SH2) domains, and (d) calcium channel antagonists. Exemplary specific restenosis-inhibiting agents include microtubule stabilizing agents such as rapamycin, mitomycin C, TAXOL®, paclitaxel (i.e., paclitaxel, paxlitaxel analogs, or paclitaxel derivatives, and mixtures thereof). For example, derivatives suitable for use in the stent include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

In some embodiments, an active agent is an anti-coagulation agent. As used herein, “anti-coagulation agent” refers to any molecule or composition that promotes blood coagulation or activates the blood coagulation cascade or a portion thereof. Exemplary anti-coagulation agents include, for example, phospholipids such as, e.g., negatively charged phospholipids; lipoproteins such as, e.g., thromboplastin, and the like; proteins such as tissue factor, activated serin proteases such as Factors IIa (thrombin), VII, VIIa, VIII, IX, IXa, Xa, XIa, XII, XIIa, von Willebrand factor (vWF), protein C, snake venoms such as PROTAC® enzyme, Ecarin, Textarin, Noscarin, Batroxobin, Thrombocytin, Russell's viper venom (RVV), and the like; polyvalent cations; calcium ions; tissue factor; silica; kaolin; bentonite;, diatomaceous earth; ellagic acid; celite; and any mixtures thereof.

In some embodiments, provided stents include for example, antibiotics. Antibiotics suitable for incorporation in stents include, but are not limited to, aminoglycosides (e.g., neomycin), ansamycins, carbacephem, carbapenems, cephalosporins (e.g., cefazolin, cefaclor, cefditoren, cefditoren, ceftobiprole), glycopeptides (e.g., vancomycin), macrolides (e.g., erythromycin, azithromycin), monobactams, penicillins (e.g., amoxicillin, ampicillin, cloxacillin, dicloxacillin, flucloxacillin), polypeptides (e.g., bacitracin, polymyxin B), quinolones (e.g., ciprofloxacin, enoxacin, gatifloxacin, ofloxacin, etc.), sulfonamides (e.g., sulfasalazine, trimethoprim, trimethoprim-sulfamethoxazole (co-trimoxazole)), tetracyclines (e.g., doxycyline, minocycline, tetracycline, etc.), chloramphenicol, lincomycin, clindamycin, ethambutol, mupirocin, metronidazole, pyrazinamide, thiamphenicol, rifampicin, thiamphenicl, dapsone, clofazimine, quinupristin, metronidazole, linezolid, isoniazid, fosfomycin, fusidic acid, β-lactam antibiotics, rifamycins, novobiocin, fusidate sodium, capreomycin, colistimethate, gramicidin, doxycycline, erythromycin, nalidixic acid, and vancomycin. For example, β-lactam antibiotics can be aziocillin, aztreonam, carbenicillin, cefoperazone, ceftriaxone, cephaloridine, cephalothin, moxalactam, piperacillin, ticarcillin and combination thereof.

In some embodiments, provided stents include for example, anti-inflammatories. Anti-inflammatory agents may include corticosteroids (e.g., glucocorticoids), cycloplegics, non-steroidal anti-inflammatory drugs (NSAIDs), immune selective anti-inflammatory derivatives (ImSAIDs), and any combination thereof. Exemplary NSAIDs include, but not limited to, celecoxib (Celebrex®); rofecoxib (Vioxx®), etoricoxib (Arcoxia®), meloxicam (Mobic®), valdecoxib, diclofenac (Voltaren®, Cataflam®), etodolac (Lodine®), sulindac (Clinori®), aspirin, alclofenac, fenclofenac, diflunisal (Dolobid®), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam (Feldene®), indomethacin (Indocin®), nabumetone (Relafen®), naproxen (Naprosyn®), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof.

In some embodiments, additives, agents, and/or functional moieties include a nitric oxide or a prodrug thereof.

In some embodiments, provided stents include, for example, polypeptides (e.g., proteins), including but are not limited to: one or more antigens, cytokines, hormones, chemokines, enzymes, and any combination thereof as an agent and/or functional group. Exemplary enzymes suitable for use herein include, but are not limited to, peroxidase, lipase, amylose, organophosphate dehydrogenase, ligases, restriction endonucleases, ribonucleases, DNA polymerases, glucose oxidase, laccase, and the like.

In some embodiments, provided stents include, for example, antibodies. Suitable antibodies for incorporation in stents include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab.

In some embodiments, an active agent is an enzyme that hydrolyzes silk fibroin. Without wishing to be bound by a theory, such enzymes can be used to control degradation of a stent after implantation into a subject. Controlled degradation of silk fibroin-fibroin based scaffolds with enzymes embedded therein is described in, for example, US Provisional Application No. 61/791,501, filed Mar. 15, 2013, content of which is incorporated herein by reference in its entirety.

In some embodiments, the bulk material of the stent can include a cell. Stent with the bulk material comprising a cell can be used for organ repair, organ replacement or regeneration. Cells amenable to be incorporated into the composition include, but are not limited to, stem cells (embryonic stem cells, mesenchymal stem cells, neural stem cells, bone-marrow derived stem cells, hematopoietic stem cells, and induced pluripotent stem cells); pluripotent cells; chrondrocytes progenitor cells; pancreatic progenitor cells; myoblasts; fibroblasts; chondrocytes; keratinocytes; neuronal cells; glial cells; astrocytes; pre-adipocytes; adipocytes; vascular endothelial cells; hair follicular stem cells; endothelial progenitor cells; mesenchymal cells; smooth muscle progenitor cells; osteocytes; parenchymal cells such as hepatocytes; pancreatic cells (including Islet cells); cells of intestinal origin; and combination thereof, either as obtained from donors, from established cell culture lines, or even before or after molecular genetic engineering. Without limitations, the cells useful for incorporation into the composition can come from any source, for example human, rat or mouse. In some embodiments, the cell can from a subject into which the stent is to be implanted.

In some embodiments, a cell is a genetically modified cell. A cell can be genetically modified to express and secrete a desired compound, e.g. a bioactive agent, a growth factor, differentiation factor, cytokines, and the like. Methods of genetically modifying cells for expressing and secreting compounds of interest are known in the art and easily adaptable by one of skill in the art.

In some embodiments, differentiated cells that have been reprogrammed into stem cells can also be used. For example, human skin cells reprogrammed into embryonic stem cells by the transduction of Oct3/4, Sox2, c-Myc and Klf4 (Junying Yu, et. al., Science , 2007, 318, 1917-1920 and Takahashi K. et. al., Cell , 2007, 131, 1-12).

In some embodiments, when using a stent with cells, it can be desirable to add other materials to promote the growth, differentiation or proliferation of the cell. Exemplary materials known to promote cell growth include, but not limited to, cell growth media, such as Dulbecco's Modified Eagle Medium (DMEM), fetal bovine serum (FBS), non-essential amino acids and antibiotics, and growth and morphogenic factors such as fibroblast growth factor (e.g., FGF 1-9), transforming growth factors (TGFs), vascular endothelial growth factor (VEGF), epidermal growth factor (EGF), platelet derived growth factor (PDGF), insulin-like growth factor (IGF-I and IGF-II), bone morphogenetic growth factors (e.g., BMPs 1-7), bone morphogenetic-like proteins (e.g., GFD-5, GFD-7, and GFD-8), transforming growth factors (e.g., TGF-α, TGF-βI-III), nerve growth factors, and related proteins. Growth factors are known in the art, see, e.g., Rosen & Thies, CELLULAR & MOL. BASIS BONE FORMATION & REPAIR (R. G. Landes Co.).

In some embodiments, cells suitable for use herein include, but are not limited to, progenitor cells or stem cells, smooth muscle cells, skeletal muscle cells, cardiac muscle cells, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, and precursor cells.

In some embodiments, provided stents include, for example, organisms, such as, a bacterium, fungus, plant or animal, or a virus. In some embodiments, an active agent may include or be selected from neurotransmitters, hormones, intracellular signal transduction agents, pharmaceutically active agents, toxic agents, agricultural chemicals, chemical toxins, biological toxins, microbes, and animal cells such as neurons, liver cells, and immune system cells. The active agents may also include therapeutic compounds, such as pharmacological materials, vitamins, sedatives, hypnotics, prostaglandins and radiopharmaceuticals.

In some embodiments, provided stents include, for example, agents useful for wound healing include stimulators, enhancers or positive mediators of the wound healing cascade which 1) promote or accelerate the natural wound healing process or 2) reduce effects associated with improper or delayed wound healing, which effects include, for example, adverse inflammation, epithelialization, angiogenesis and matrix deposition, and scarring and fibrosis.

In some embodiments, provided stents include, for example, an optically or electrically active agent, including but not limited to, chromophores; light emitting organic compounds such as luciferin, carotenes; light emitting inorganic compounds, such as chemical dyes; light harvesting compounds such as chlorophyll, bacteriorhodopsin, protorhodopsin, and porphyrins; light capturing complexes such as phycobiliproteins; and related electronically active compounds; and combinations thereof.

Without wishing to be bound by a theory, incorporating an active agent in a bulk material of a stent enables delivery of an active agent in a controlled released manner. Maintaining an active agent in an active form throughout in the silk fibroin-fibroin matrix enables it to be active upon release from the stent. Controlled release of active agent permits active agent to be released sustainably over time, with controlled release kinetics. In some embodiments, an active agent is delivered continuously to the site where treatment is needed, for example, over several weeks. Controlled release over time, for example, over several days or weeks, or longer, permits continuous delivery of the bioactive agent to obtain preferred treatments. In some embodiments, controlled delivery is advantageous because it protects bioactive agents from degradation in vivo in body fluids and tissue, for example, by proteases.

Controlled release of an active agent from the stent can be designed to occur over time, for example, over 12 hours or 24 hours. Time of release may be selected, for example, to occur over a time period of about 12 hours to 24 hours; about 12 hours to 42 hours; or, e.g., about 12 to 72 hours. In another embodiment, release can occur for example on the order of about 1 day to 15 days. Controlled release time can be selected based on the condition treated. For example, longer times can be more effective for wound healing, whereas shorter delivery times can be more useful for some cardiovascular applications.

Controlled release of an active agent from a stent in vivo can occur, for example, in the amount of about 1 ng to 1 mg/day. In some embodiments, controlled release can occur in the amount of about 50 ng to 500 ng/day, about 75 ng to 250 ng/day, about 100 ng to 200 ng/day, or about 125 ng to 175 ng/day.

In some embodiments, provided silk fibroin tracheal stents include additives, agents, and/or functional moieties at a total amount from about 0.01 wt % to about 99 wt %, from about 0.01 wt % to about 70 wt %, from about 5 wt % to about 60 wt %, from about 10 wt % to about 50 wt %, from about 15 wt % to about 45 wt %, or from about 20 wt % to about 40 wt %, of the total silk composition. In some embodiments, ratio of silk fibroin to additive in the composition can range from about 1000:1 (w/w) to about 1:1000 (w/w), from about 500:1 (w/w) to about 1:500 (w/w), from about 250:1 (w/w) to about 1:250 (w/w), from about 200:1 (w/w) to about 1:200 (w/w), from about 25:1 (w/w) to about 1:25 (w/w), from about 20:1 (w/w) to about 1:20 (w/w), from about 10:1 (w/w) to about 1:10 (w/w), or from about 5:1 (w/w) to about 1:5 (w/w).

In some embodiments, provided silk fibroin tracheal stents include additives, agents, and/or functional moieties at a molar ratio relative to polymer (i.e., a polymer:additive ratio) of, e.g., at least 1000:1, at least 900:1, at least 800:1, at least 700:1, at least 600:1, at least 500:1, at least 400:1, at least 300:1, at least 200:1, at least 100:1, at least 90:1, at least 80:1, at least 70:1, at least 60:1, at least 50:1, at least 40:1, at least 30:1, at least 20:1, at least 10:1, at least 7:1, at least 5:1, at least 3:1, at least 1:1, at least 1:3, at least 1:5, at least 1:7, at least 1:10, at least 1:20, at least 1:30, at least 1:40, at least 1:50, at least 1:60, at least 1:70, at least 1:80, at least 1:90, at least 1:100, at least 1:200, at least 1:300, at least 1:400, at least 1:500, at least 600, at least 1:700, at least 1:800, at least 1:900, or at least 1:100.

In some embodiments, moiety polymer:additive ratio is, e.g., at most 1000:1, at most 900:1, at most 800:1, at most 700:1, at most 600:1, at most 500:1, at most 400:1, at most 300:1, at most 200:1, 100:1, at most 90:1, at most 80:1, at most 70:1, at most 60:1, at most 50:1, at most 40:1, at most 30:1, at most 20:1, at most 10:1, at most 7:1, at most 5:1, at most 3:1, at most 1:1, at most 1:3, at most 1:5, at most 1:7, at most 1:10, at most 1:20, at most 1:30, at most 1:40, at most 1:50, at most 1:60, at most 1:70, at most 1:80, at most 1:90, at most 1:100, at most 1:200, at most 1:300, at most 1:400, at most 1:500, at most 1:600, at most 1:700, at most 1:800, at most 1:900, or at most 1:1000.

In some embodiments, moiety polymer:additive ratio is, e.g., from about 1000:1 to about 1:1000, from about 900:1 to about 1:900, from about 800:1 to about 1:800, from about 700:1 to about 1:700, from about 600:1 to about 1:600, from about 500:1 to about 1:500, from about 400:1 to about 1:400, from about 300:1 to about 1:300, from about 200:1 to about 1:200, from about 100:1 to about 1:100, from about 90:1 to about 1:90, from about 80:1 to about 1:80, from about 70:1 to about 1:70, from about 60:1 to about 1:60, from about 50:1 to about 1:50, from about 40:1 to about 1:40, from about 30:1 to about 1:30, from about 20:1 to about 1:20, from about 10:1 to about 1:10, from about 7:1 to about 1:7, from about 5:1 to about 1:5, from about 3:1 to about 1:3, or about 1:1.

In some embodiments, a ratio of silk fibroin to a total amount of additive, agent, and/or functional moiety in a bulk material can range from 100:1 to 1:100. For example, the ratio of silk fibroin to additive can range from 50:1 to 1:50, from 25:1 to 1:25, from 20:1 to 1: 20, from 15:1 to 1:15, from 10:1 to 1:10, or from 5:1 to 1:5. In some embodiments, a ratio of silk fibroin to additive, agent, and/or functional moiety can be from 5:1 to 1:1. In one embodiment, a ratio of silk fibroin to additive, agent, and/or functional moiety can be 3:1. A ratio can be molar ratio, weight ratio, or volume ratio.

A total amount of active agent in a bulk material can be from about 0.1 wt % to about 0.99 wt %, from about 0.1 wt % to about 70 wt %, from about 5 wt % to about 60 wt %, from about 10 wt % to about 50 wt %, from about 15 wt % to about 45 wt %, or from about 20 wt % to about 40 wt %, of a total weight of bulk material.

### Nucleic Acids

In some embodiments, provided stents include additives, for example, nucleic acid agents. In some embodiments, a stent may release nucleic acid agents. In some embodiments, a nucleic acid agent is or includes a therapeutic agent. In some embodiments, a nucleic acid agent is or includes a diagnostic agent. In some embodiments, a nucleic acid agent is or includes a prophylactic agent.

It would be appreciate by those of ordinary skill in the art that a nucleic acid agent can have a length within a broad range. In some embodiments, a nucleic acid agent has a nucleotide sequence of at least about 40, for example at least about 60, at least about 80, at least about 100, at least about 200, at least about 500, at least about 1000, or at least about 3000 nucleotides in length. In some embodiments, a nucleic acid agent has a length from about 6 to about 40 nucleotides. For example, a nucleic acid agent may be from about 12 to about 35 nucleotides in length, from about 12 to about 20 nucleotides in length or from about 18 to about 32 nucleotides in length.

In some embodiments, nucleic acid agents may be or include deoxyribonucleic acids (DNA), ribonucleic acids (RNA), peptide nucleic acids (PNA), morpholino nucleic acids, locked nucleic acids (LNA), glycol nucleic acids (GNA), threose nucleic acids (TNA), and/or combinations thereof.

In some embodiments, a nucleic acid has a nucleotide sequence that is or includes at least one protein-coding element. In some embodiments, a nucleic acid has a nucleotide sequence that is or includes at least one element that is a complement to a protein-coding sequence. In some embodiments, a nucleic acid has a nucleotide sequence that includes one or more gene expression regulatory elements (e.g., promoter elements, enhancer elements, splice donor sites, splice acceptor sites, transcription termination sequences, translation initiation sequences, translation termination sequences, etc.). In some embodiments, a nucleic acid has a nucleotide sequence that includes an origin of replication. In some embodiments, a nucleic acid has a nucleotide sequence that includes one or more integration sequences. In some embodiments, a nucleic acid has a nucleotide sequence that includes one or more elements that participate in intra- or inter-molecular recombination (e.g., homologous recombination). In some embodiments, a nucleic acid has enzymatic activity. In some embodiments, a nucleic acid hybridizes with a target in a cell, tissue, or organism. In some embodiments, a nucleic acid acts on (e.g., binds with, cleaves, etc.) a target inside a cell. In some embodiments, a nucleic acid is expressed in a cell after release from a provided composition. In some embodiments, a nucleic acid integrates into a genome in a cell after release from a provided composition.

In some embodiments, nucleic acid agents have single-stranded nucleotide sequences. In some embodiments, nucleic acid agents have nucleotide sequences that fold into higher order structures (e.g., double and/or triple-stranded structures). In some embodiments, a nucleic acid agent is or includes an oligonucleotide. In some embodiments, a nucleic acid agent is or includes an antisense oligonucleotide. Nucleic acid agents may include a chemical modification at the individual nucleotide level or at the oligonucleotide backbone level, or it may have no modifications.

In some embodiments of the present disclosure, a nucleic acid agent is an siRNA agent. Short interfering RNA (siRNA) includes an RNA duplex that is approximately 19 basepairs long and optionally further includes one or two single-stranded overhangs. An siRNA may be formed from two RNA molecules that hybridize together, or may alternatively be generated from a single RNA molecule that includes a self-hybridizing portion. It is generally preferred that free 5′ ends of siRNA molecules have phosphate groups, and free 3′ ends have hydroxyl groups. The duplex portion of an siRNA may, but typically does not, contain one or more bulges consisting of one or more unpaired nucleotides. One strand of an siRNA includes a portion that hybridizes with a target transcript. In certain preferred embodiments of the disclosure, one strand of the siRNA is precisely complementary with a region of the target transcript, meaning that the siRNA hybridizes to the target transcript without a single mismatch. In other embodiments of the disclosure one or more mismatches between the siRNA and the targeted portion of the target transcript may exist. In most embodiments of the disclosure in which perfect complementarity is not achieved, it is generally preferred that any mismatches be located at or near the siRNA termini.

Short hairpin RNA refers to an RNA molecule comprising at least two complementary portions hybridized or capable of hybridizing to form a double-stranded (duplex) structure sufficiently long to mediate RNAi (typically at least 19 base pairs in length), and at least one single-stranded portion, typically between approximately 1 and 10 nucleotides in length that forms a loop. The duplex portion may, but typically does not, contain one or more bulges consisting of one or more unpaired nucleotides. As described further below, shRNAs are thought to be processed into siRNAs by the conserved cellular RNAi machinery. Thus shRNAs are precursors of siRNAs and are, in general, similarly capable of inhibiting expression of a target transcript.

In describing siRNAs it will frequently be convenient to refer to sense and antisense strands of the siRNA. In general, the sequence of the duplex portion of the sense strand of the siRNA is substantially identical to the targeted portion of the target transcript, while the antisense strand of the siRNA is substantially complementary to the target transcript in this region as discussed further below. Although shRNAs contain a single RNA molecule that self-hybridizes, it will be appreciated that the resulting duplex structure may be considered to include sense and antisense strands or portions. It will therefore be convenient herein to refer to sense and antisense strands, or sense and antisense portions, of an shRNA, where the antisense strand or portion is that segment of the molecule that forms or is capable of forming a duplex and is substantially complementary to the targeted portion of the target transcript, and the sense strand or portion is that segment of the molecule that forms or is capable of forming a duplex and is substantially identical in sequence to the targeted portion of the target transcript.

For purposes of description, the discussion below may refer to siRNA rather than to siRNA or shRNA. However, as will be evident to one of ordinary skill in the art, teachings relevant to the sense and antisense strand of an siRNA are generally applicable to the sense and antisense portions of the stem portion of a corresponding shRNA. Thus in general the considerations below apply also to shRNAs.

An siRNA agent is considered to be targeted to a target transcript for the purposes described herein if 1) the stability of the target transcript is reduced in the presence of the siRNA or shRNA as compared with its absence; and/or 2) the siRNA or shRNA shows at least about 90%, more preferably at least about 91%, 92%, 93%, 94%, 95%, 96%, 97%, 98%, 99%, or 100% precise sequence complementarity with the target transcript for a stretch of at least about 15, more preferably at least about 17, yet more preferably at least about 18 or 19 to about 21-23 nucleotides; and/or 3) one strand of the siRNA or one of the self-complementary portions of the shRNA hybridizes to the target transcript under stringent conditions for hybridization of small (<50 nucleotide) RNA molecules in vitro and/or under conditions typically found within the cytoplasm or nucleus of mammalian cells. Since the effect of targeting a transcript is to reduce or inhibit expression of the gene that directs synthesis of the transcript, an siRNA, shRNA, targeted to a transcript is also considered to target the gene that directs synthesis of the transcript even though the gene itself (i.e., genomic DNA) is not thought to interact with the siRNA, shRNA, or components of the cellular silencing machinery. Thus in some embodiments, an siRNA, shRNA, that targets a transcript is understood to target the gene that provides a template for synthesis of the transcript.

In some embodiments, an siRNA agent can inhibit expression of a polypeptide (e.g., a protein). Exemplary polypeptides include, but are not limited to, matrix metallopeptidase 9 (MMP-9), neutral endopeptidase (NEP) and protein tyrosine phosphatase 1B (PTP1B).

### Growth Factor

In some embodiments, provided stents include additives, for example, growth factor. In some embodiments, a stent may release growth factor. In some embodiments, a stent may release multiple growth factors. In some embodiments growth factor known in the art include, for example, adrenomedullin, angiopoietin, autocrine motility factor, basophils, brain-derived neurotrophic factor, bone morphogenetic protein, colony-stimulating factors, connective tissue growth factor, endothelial cells, epidermal growth factor, erythropoietin, fibroblast growth factor, fibroblasts, glial cell line-derived neurotrophic factor, granulocyte colony stimulating factor, granulocyte macrophage colony stimulating factor, growth differentiation factor-9, hepatocyte growth factor, hepatoma-derived growth factor, insulin-like growth factor, interleukins, keratinocyte growth factor, keratinocytes, lymphocytes, macrophages, mast cells, myostatin, nerve growth factor, neurotrophins, platelet-derived growth factor, placenta growth factor, osteoblasts, platelets, proinflammatory, stromal cells, T-lymphocytes, thrombopoietin, transforming growth factor alpha, transforming growth factor beta, tumor necrosis factor-alpha, vascular endothelial growth factor and combinations thereof.

In some embodiments, provided stents include additives, for example, that are particularly useful for healing. Exemplary agents useful as growth factor for defect repair and/or healing can include, but are not limited to, growth factors for defect treatment modalities now known in the art or later-developed; exemplary factors, agents or modalities including natural or synthetic growth factors, cytokines, or modulators thereof to promote bone and/or tissue defect healing. Suitable examples may include, but not limited to 1) topical or dressing and related therapies and debriding agents (such as, for example, Santyl® collagenase) and Iodosorb® (cadexomer iodine); 2) antimicrobial agents, including systemic or topical creams or gels, including, for example, silver-containing agents such as SAGs (silver antimicrobial gels), (CollaGUARD™, Innocoll, Inc) (purified type-I collagen protein based dressing), CollaGUARD Ag (a collagen-based bioactive dressing impregnated with silver for infected wounds or wounds at risk of infection), DermaSIL™ (a collagen-synthetic foam composite dressing for deep and heavily exuding wounds); 3) cell therapy or bioengineered skin, skin substitutes, and skin equivalents, including, for example, Dermograft (3-dimensional matrix cultivation of human fibroblasts that secrete cytokines and growth factors), Apligraf® (human keratinocytes and fibroblasts), Graftskin® (bilayer of epidermal cells and fibroblasts that is histologically similar to normal skin and produces growth factors similar to those produced by normal skin), TransCyte (a Human Fibroblast Derived Temporary Skin Substitute) and Oasis® (an active biomaterial that includes both growth factors and extracellular matrix components such as collagen, proteoglycans, and glycosaminoglycans); 4) cytokines, growth factors or hormones (both natural and synthetic) introduced to the wound to promote wound healing, including, for example, NGF, NT3, BDGF, integrins, plasmin, semaphoring, blood-derived growth factor, keratinocyte growth factor, tissue growth factor, TGF-alpha, TGF-beta, PDGF (one or more of the three subtypes may be used: AA, AB, and B), PDGF-BB, TGF-beta 3, factors that modulate the relative levels of TGFβ3, TGFβ1, and TGFβ2 (e.g., Mannose-6-phosphate), sex steroids, including for example, estrogen, estradiol, or an oestrogen receptor agonist selected from the group consisting of ethinyloestradiol, dienoestrol, mestranol, oestradiol, oestriol, a conjugated oestrogen, piperazine oestrone sulphate, stilboestrol, fosfesterol tetrasodium, polyestradiol phosphate, tibolone, a phytoestrogen, 17-beta-estradiol; thymic hormones such as Thymosin-beta-4, EGF, HB-EGF, fibroblast growth factors (e.g., FGF1, FGF2, FGF7), keratinocyte growth factor, TNF, interleukins family of inflammatory response modulators such as, for example, IL-10, IL-1, IL-2, IL-6, IL-8, and IL-10 and modulators thereof; INFs (INF-alpha, -beta, and -delta); stimulators of activin or inhibin, and inhibitors of interferon gamma prostaglandin E2 (PGE2) and of mediators of the adenosine 3′,5′-cyclic monophosphate (cAMP) pathway; adenosine A1 agonist, adenosine A2 agonist or 5) other agents useful for wound healing, including, for example, both natural or synthetic homologues, agonist and antagonist of VEGF, VEGFA, IGF; IGF-1, proinflammatory cytokines, GM-CSF, and leptins and 6) IGF-1 and KGF cDNA, autologous platelet gel, hypochlorous acid (Sterilox® lipoic acid, nitric oxide synthase3, matrix metalloproteinase 9 (MMP-9), CCT-ETA, alphavbeta6 integrin, growth factor-primed fibroblasts and Decorin, silver containing wound dressings, Xenaderm™, papain wound debriding agents, lactoferrin, substance P, collagen, and silver-ORC, placental alkaline phosphatase or placental growth factor, modulators of hedgehog signaling, modulators of cholesterol synthesis pathway, and APC (Activated Protein C), keratinocyte growth factor, TNF, Thromboxane A2, NGF, BMP bone morphogenetic protein, CTGF (connective tissue growth factor), wound healing chemokines, decorin, modulators of lactate induced neovascularization, cod liver oil, placental alkaline phosphatase or placental growth factor, and thymosin beta 4. In certain embodiments, one, two three, four, five or six agents useful for wound healing may be used in combination. More details can be found in U.S. Pat. No. 8,247,384, the contents of which are incorporated herein by reference.

It is to be understood that agents useful for growth factor for healing (including for example, growth factors and cytokines) above encompass all naturally occurring polymorphs (for example, polymorphs of the growth factors or cytokines). Also, functional fragments, chimeric proteins comprising one of said agents useful for wound healing or a functional fragment thereof, homologues obtained by analogous substitution of one or more amino acids of the wound healing agent, and species homologues are encompassed. It is contemplated that one or more agents useful for wound healing may be a product of recombinant DNA technology, and one or more agents useful for wound healing may be a product of transgenic technology. For example, platelet derived growth factor may be provided in the form of a recombinant PDGF or a gene therapy vector comprising a coding sequence for PDGF.

In some embodiments, the active agent is a growth factor or cytokine. A non-limiting list of growth factors and cytokines includes, but is not limited, to stem cell factor (SCF), granulocyte-colony stimulating factor (G-CSF), granulocyte-macrophage stimulating factor (GM-CSF), stromal cell-derived factor-1, steel factor, VEGF, TGFβ, platelet derived growth factor (PDGF), angiopoeitins (Ang), epidermal growth factor (EGF), bFGF, HNF, NGF, bone morphogenic protein (BMP), fibroblast growth factor (FGF), hepatocye growth factor, insulin-like growth factor (IGF-1), interleukin (IL)-3, IL-la, IL-1(3, IL-6, IL-7, IL-8, IL-11, and IL-13, colony-stimulating factors, thrombopoietin, erythropoietin, fit3-ligand, and tumor necrosis factors (TNFα and TNFβ). Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, P A: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

In some embodiments, the active agent can be selected from anti-infectives such as antibiotics and antiviral agents; chemotherapeutic agents (i.e. anticancer agents); anti-rejection agents; anti-proliferative agents; analgesics and analgesic combinations; anti-inflammatory agents; erythropoietin (EPO); interferon α and γ; interleukins; tumor necrosis factor α and β; insulin, antibiotics; adenosine; cytokines; integrins; selectins; cadherins; insulin; hormones such as steroids; cytotoxins; prodrugs; immunogens; or lipoproteins.

In some embodiments, provided stents include additives, for example, that are particularly useful as diagnostic agents. In some embodiments, diagnostic agents include gases; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MM include gadolinium chelates, as well as iron, magnesium, manganese, copper, and chromium. Examples of materials useful for CAT and x-ray imaging include iodine-based materials.

In some embodiments, provided stents include additives, for example, radionuclides that are particularly useful as therapeutic and/or diagnostic agents. Among the radionuclides used, gamma-emitters, positron-emitters, and X-ray emitters are suitable for diagnostic and/or therapy, while beta emitters and alpha-emitters may also be used for therapy. Suitable radionuclides for forming thermally-responsive conjugates in accordance with the disclosure include, but are not limited to, ¹²³I, ¹²⁵I, ¹³⁰I, ¹³¹I, ¹³³I, ¹³⁵I, ⁴⁷Sc, ⁷²Se, ⁷²Se, ⁹⁰Y, ⁸⁸Y, ⁹⁷Ru, ¹⁰⁰Pd, ¹⁰¹mRh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁷Cu, ⁷⁵Br, ⁷⁷Br, ⁹⁹mTc, ¹⁴C, ¹³N, ¹⁵O, ³²P, ³³P, and ¹⁸F. In some embodiments, a diagnostic agent may be a fluorescent, luminescent, or magnetic moiety.

Fluorescent and luminescent moieties include a variety of different organic or inorganic small molecules commonly referred to as “dyes,” “labels,” or “indicators.” Examples include fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin and derivatives thereof. Numerous fluorescent and luminescent dyes and proteins are known in the art (see, e.g., U.S. Patent Application Publication No.: 2004/0067503; Valeur, B., “Molecular Fluorescence: Principles and Applications,” John Wiley and Sons, 2002; Handbook of Fluorescent Probes and Research Products, Molecular Probes, 9^{th} edition, 2002; and The Handbook—A Guide to Fluorescent Probes and Labeling Technologies, Invitrogen, 10^{th} edition, available at the Invitrogen web site; both of which are incorporated herein by reference).

### Silk Fibroin Tracheal Stent Designs

Stent families, which vary by deployment mechanism complement each other and provide options for a broader range of stenting applications. Designs are composed of a bulk material. In some embodiments, other features of silk are retained, including the ability to load and deliver therapeutic compounds and 100% degradability of the stent material over time. In some embodiments, a bulk material consists of a silk:glycerol blend in a dry weight ratio of 75:25. In some embodiments, a bulk material is fabricated as described below.

In some embodiments, provided silk fibroin tracheal stents are tubular in shape. In some embodiments, provided stents are concentric. In some embodiments, provided silk fibroin tracheal stents are tubular and having a range of about 120° to about 360°. In some embodiments, provided silk fibroin tracheal stents are tubular and range of about 200° to about 340°, about 220° to about 320°, about 240° to about 300°, or about 260° to about 280°.

In some embodiments, provided silk fibroin tracheal stents and concentric with a circular dimension of about 360°, about 350°, about 340°, about 330°, about 320°, about 310°, about 300°, about 290°, about 280°, about 270°, about 260°, about 250°, about 240°, about 230°, about 220°, about 210°, about 200°, about 190°, about 180°, about 170°, about 160°, about 150°, about 140°, about 130°, or about 120°.

In some embodiments, when a stent is characterized by a circular dimension having a range of about 120° to about 360° such a stent provides ample radial support. In some embodiments, silk fibroin tracheal stents are characterized by a circular dimension having a range of about 120° to about 360° provides ample radial support without necessitating a more invasive surgery to extent the stent around the posterior trachea.

In some embodiments, provided tracheal stent graphs include a radial opening having a dimension between about 0° and about 240°. In some embodiments, provided tracheal stent graphs are substantially cylindrical and include a radial opening between about 0° and about 240°. In some embodiments, a radial opening has a dimension in a range of about 0° to about 240°, about 10° to about 230°, about 20° to about 220°, about 30° to about 210°, about 40° to about 200°, about 50° to about 190°, about 60° to about 180°, about 70° to about 170°, about 80° to about 160°, about 90° to about 150°, about 100° to about 140°, or about 110° to about 130°.

In some embodiments, provided tracheal stent graphs include a radial opening having a dimension of about 0°, about 10°, about 20°, about 30°, about 40°, about 50°, about 60°, about 70°, about 80°, about 90°, about 100°, about 110°, about 120°, about 130°, about 140°, about 150°, about 160°, about 170°, about 180°, about 190°, about 200°, about 210°, about 220°, about 230°, or about 240°.

The stent disclosed herein can include any desired mechanical stiffness. For example, the stent can include an average mechanical stiffness of about 0.01 kN/m² to about 100 kN/m². In some embodiments, the stent can include an average mechanical stiffness of from about 0.05 kN/m² to about 75 kN/m², from about 0.1 kN/m² to about 50 kN/m², from about 0.25 kN/m² to about 25 kN/m², from about 0.5 kN/m² to about 10 kN/m², or from about 0.75 kN/m² to about 2 kN/m². In one embodiment, the stent has an average mechanical stiffness of about 1.2 kN/m².

The radial strength of the stent can also be optimized for any desired application. For example, the stent can have an average radial strength of from about 100 mmHg to about 1000 mmHg. In some embodiments, the stent has an average radial strength of from about 75 mmHg to about 750 mmHg, from about 50 mmHg to about 600 mmHg, from about 100 mmHg to about 500 mmHg, from about 150 mmHg to about 450 mmHg, from about 200 mmHg to about 450 mmHg, or from about 250 mmHg to about 350 mmHg. In some embodiments, the stent has an average radial strength of about 300 mmHg.

Compressive toughness is the capacity of a material to resist fracture when subjected to axially directed pushing forces. By definition, the compressive toughness of a material is the ability to absorb mechanical (or kinetic) energy up to the point of failure. Toughness is measured in units of joules per cubic meter (Jm⁻³) and can be measured as the area under a stress-strain curve. In some embodiments, the stent has a compressive toughness of about 1 kJ m⁻³ to about 20 kJm⁻³ or about 1 kJm⁻³ to about 5 kJm⁻³ at 6% strain as measured by the J-integral method.

Compressive strength is the capacity of a material to withstand axially directed pushing forces. By definition, the compressive strength of a material is that value of uniaxial compressive stress reached when the material fails completely. A stress-strain curve is a graphical representation of the relationship between stress derived from measuring the load applied on the sample (measured in MPa) and strain derived from measuring the displacement as a result of compression of the sample. The ultimate compressive strength of the material can depend upon the target site of implantation. In some embodiments, the stent include a compressive strength (stress to yield point) of approximately 1 MPa to approximately 10 MPa.

Compressive elastic modulus is the mathematical description of the tendency of a material to be deformed elastically (i.e. non-permanently) when a force is applied to it. The Young's modulus (E) describes tensile elasticity, or the tendency of a material to deform along an axis when opposing forces are applied along that axis; it is defined as the ratio of tensile stress to tensile strain (measured in MPa) and is otherwise known as a measure of stiffness of the material. The elastic modulus of an object is defined as the slope of the stress-strain curve in the elastic deformation region. The stent can include a compressive elastic modulus of between approximately 1 MPa and approximately 30 MPa at 5% strain.

In some embodiments, the stent can be bioresorbed after implantation into a subject. As used herein, the term “bioresorbed” or “bioresorption” refers to infiltration of endogenous tissue or cells into an implanted structure, e.g., stent, which permits integration of the implantable structure and tissues, where one or more components of the implanted structure is replaced by new tissue. For example, the stent can degrade as tissue surrounding the target site remodels or regenerates.

In some embodiments, the cylindrical body portion of the stent can be a multilayered cylindrical body portion. If a multilayered stent includes an additive and/or active agent, different layers of the body can includes same or different additive or active agents. For example, some layers can include a first additive (or active agent) and some other layers can include a second additive (or active agent). In some embodiments, the outermost layer includes no active agent. The number of layers in the multilayered cylindrical body portion of the stent can be any desired number. For example, the multilayered cylindrical body portion of the stent can include from 1 to 100, 1 to 75, 1 to 50, 1 25, or 1 to 20 layers.

Without limitations, thickness of each layer can range independently from nanometers to millimeters. For example, thickness of layer can be lnm to 1000 nm, 1 nm to 500 nm, 1 nm to 250 nm, 1 nm to 100 nm, lnm to 50 nm, 1 nm to 25 nm, 1 nm to 10 nm, 1 μm to 1000 μm, 1 μm to 500 μm, 1 μm to 250 μm, 1 μm to 100 μm, 1 μm to 50 μm, or 1 μm to 25 μm. Further all layers can be of the same thickness, all of different thickness, or some of same and some of different thickness.

The stent designs according to the present disclosure can also incorporate other features of silk fibroin and silk fibroin based polymers, including the ability to load and deliver therapeutic compounds and up to 100% degradability of the stent material over time within the body to support such delivery.

The silk fibroin sheet for the ratcheting stent design (e.g., stents 100, 200, 300, 400) can be made using any method known in the art for preparing films, e.g., films comprising silk fibroin. As used herein, the term “film” refers to a flat or tubular flexible structure. It is to be noted that the term “film” is used in a generic sense to include a web, film, sheet, laminate, or the like. In some embodiments, the film is a patterned film, e.g., nanopatterned film. Exemplary methods for preparing films comprising silk fibroin are described in, for example, WO 2004/000915 and WO 2005/012606, content of both of which is incorporated herein by reference in its entirety.

### Methods

In some embodiments, methods of manufacturing silk fibroin based tracheal stent graphs are provided. In some embodiments, provided methods of manufacturing include providing a silk fibroin solution. In some embodiments, provided silk fibroin solutions have a concentration of about 2% to about 30%.

In some embodiments, provided methods of manufacturing include adding a silk fibroin solution to a mold.

Fabrication of stents from bulk material solutions required different methods described below. After fabrication, stents were annealed in a humid environment for 6 hours at 80° C., to induce β-sheet formation, increase the mesh crystallinity and thereby improve mesh mechanical properties, resiliency, and water insolubility. Alternatively, following published protocols, stents can be submerged in 99.9% (w/v) methanol for 5 minutes to induce crystallinity.

In some embodiments, provided methods of manufacturing include freezing a silk fibroin solution to form a tracheal stent. In some embodiments, provided methods of manufacturing include porogen leaching a silk fibroin solution to form a tracheal stent. In some embodiments, provided methods of manufacturing include gel spinning a silk fibroin solution to form a tracheal stent. In some embodiments, provided methods of manufacturing include micromolding a silk fibroin solution to form a tracheal stent.

In some embodiments, a step of freezing includes lowering a temperature of the solution to about −45° C. at a rate of about 0.1° C./minute to about 5° C./minute. In some embodiments a step of freezing includes drying a silk fibroin solution under vacuum.

In some embodiments, methods further include a step of submerging a tracheal stent in methanol.

In some embodiments, methods further include a step of autoclaving a tracheal stent.

In some embodiments, methods further include a step of water annealing a tracheal stent.

In some embodiments, methods include a step of encapsulating or embedding an additive, agent or functional moiety a provided silk fibroin tracheal stent. In some embodiments, a step of encapsulating or embedding includes blending or mixing an additive, agent or functional moiety in a silk fibroin solution. In some embodiments, methods include a step of coating an additive, agent or functional moiety on a surface of a provided silk fibroin tracheal stent.

In some embodiments, an additive, agent, or functional moiety is or includes an active agent, a plasticizer, silk fibroin fibers, a therapeutic, or combinations thereof. In some embodiments, a plasticizer is or includes 1,2-butylene glycol; 2-amino-2-methyl-1,3-propanediol; 2,3-butylene glycol; allyl glycolate; butyl lactate; diethanolamine; diethylene glycol monoethyl ether; ethyl glycolate; ethyl lactate; ethylene glycol; ethylene glycol monoethyl ether; glycerol; glyceryl monostearate; monoethanolamine; monisopropanolamine; monopropylene glycol monoisopropyl ether; polyethylene glycol; polyethylene oxides; propylene glycol; propylene glycol monoethyl ether; sorbitol lactate; styrene glycol; triethanolamine; triethylenetetramine; or combinations thereof.

In some embodiments, additives, agents, or functional moieties are or include antibodies or fragments or portions thereof; antibiotics or antimicrobial compounds; antigens or epitopes; anti-proliferative agents; aptamers; biopolymers; cell adhesion proteins, cell attachment mediators; cleavable cross-linkers; cytokines; enzymes; growth factors or recombinant growth factors and fragments and variants thereof; hormone antagonists; hormones; nanoparticles; nucleic acid analogs; nucleic acids; nucleotides; oligonucleotides; peptide nucleic acids (PNA); peptides; proteins; radiopaque markers; small molecules; soluble drugs, therapeutic agents and prodrugs; toxins; or combinations thereof.

In some embodiments, additives, agents, or functional moieties are or include cells. In some embodiments, cells are viable cells. In some embodiments, viable cells are cells derived from a subject. In some embodiments, methods include a step of encapsulating or embedding viable cells. In some embodiments, encapsulating or embedding includes blending or mixing viable cells with a silk fibroin solution.

In some embodiments, provided methods of manufacturing a silk fibroin based tracheal stent graph include passing a silk fibroin solution through a 3D printer to generate a tracheal stent graph.

In some embodiments, methods of deploying silk fibroin based tracheal stent graphs are provided. In some embodiments, methods of deploying include grafting a silk fibroin based tracheal stent graph to an external site of a subject's trachea. In some embodiments, methods of deploying a silk fibroin based tracheal stent graph includes implanting a silk fibroin based tracheal stent graph in a body lumen and externally affixing it to a tracheal wall for treatment of suprasomal collapse, tracheal malacia, or tracheal stenosis.

In some embodiments, methods of deploying include ratcheting of provided silk fibroin based tracheal stents.

In some embodiments, methods of deploying provided silk fibroin based tracheal stents include a ratcheting design for increasing stent diameter.

In some embodiments, provided methods include altering a conformation of silk fibroin by water annealing. Without wishing to be bound by a theory, it is believed that physical temperature-controlled water vapor annealing (TCWVA) provides a simple and effective method to obtain refined control of the molecular structure of silk fibroin biomaterials. The silk fibroin materials can be prepared with control of crystallinity, from a low content, using conditions at 4° C. (α helix (alpha-helix) dominated silk fibroin I structure), to highest content of ˜60% crystallinity at 100° C. (β-sheet (beta-sheet) dominated silk fibroin II structure). This physical approach covers the range of structures previously reported to govern crystallization during the fabrication of silk fibroin materials, yet offers a simpler, green chemistry, approach with tight control of reproducibility. Temperature controlled water vapor annealing is described, for example, in Hu et al., Regulation of Silk fibroin Material Structure By Temperature Controlled Water Vapor Annealing, Biomacromolecules, 2011, 12(5): 1686-1696, content of which is incorporated herein by reference in its entirety.

In some embodiments, altering a conformation of silk fibroin can be induced by immersing in alcohol, e.g., methanol, ethanol, etc. In some embodiments, alcohol concentration can be at least 10%, at least 20%, at least 30%, at least 40%, at least 50%, at least 60%, at least 70%, at least 80%, at least 90% or 100%. In some embodiments, alcohol concentration is 100%. If the alteration in the conformation is by immersing in a solvent, the silk fibroin composition can be washed, e.g., with solvent/water gradient to remove any of the residual solvent that is used for the immersion. The washing can be repeated one, e.g., one, two, three, four, five, or more times.

In some embodiments, altering a conformation of silk fibroin can be induced with sheer stress. In some embodiments, sheer stress can be applied, for example, by passing the silk fibroin composition through a needle. Other methods of inducing conformational changes include applying an electric field, applying pressure, or changing the salt concentration.

In some embodiments, treatment time for inducing the conformational change can be any period of time to provide a desired silk fibroin II (beta-sheet crystallinity) content. In some embodiments, treatment time can range from about 1 hour to about 12 hours, from about 1 hour to about 6 hours, from about 1 hour to about 5 hours, from about 1 hour to about 4 hours, or from about 1 hour to about 3 hours. In some embodiments, the sintering time can range from about 2 hours to about 4 hours or from 2.5 hours to about 3.5 hours.

In some embodiments, when inducing a conformational change by solvent immersion, treatment time can range from minutes to hours. For example, immersion in the solvent can be for a period of at least about 15 minutes, at least about 30 minutes, at least about 1 hour, at least about 2 hours, at least 3 hours, at least about 6 hours, at least about 18 hours, at least about 12 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some embodiments, immersion in the solvent can be for a period of about 12 hours to about seven days, about 1 day to about 6 days, about 2 to about 5 days, or about 3 to about 4 days. In one embodiment, immersion in the solvent can be for a period of about minutes.

Without limitation, silk fibroin tracheal stents can include a silk fibroin II beta-sheet crystallinity content of at least about 5%, at least about 10%, at least about 20%, at least about 30%, at least about 40%, at least about 50%, at least about 60%, at least about 70%, at least about 80%, at least about 90%, or at least about 95% but not 100% (i.e., all the silk fibroin is present in a silk fibroin II beta-sheet conformation). In some embodiments, silk fibroin in a stent is present completely in a silk fibroin II beta-sheet conformation, i.e., 100% silk fibroin II beta-sheet crystallinity.

In some embodiments, a stent can be porous, i.e., a bulk material can include pores, such as micropores. For example, the bulk material of the stent can have a porosity of at least about 10%, at least about 20%, at least about 30%, at least about 40%, at least about 50%, at least about 60%, at least about 70%, at least about 80%, at least about 90%, or higher. One of skill in the art can adjust the porosity accordingly, based on a number of factors such as, but not limited to, desired physical or mechanical properties of the stent, release rates, molecular size and/or diffusion coefficient of the molecule distributed in the bulk material, and/or concentrations, amounts of silk fibroin in the bulk material. As used herein, the term “porosity” is a measure of void spaces in a material and is a fraction of volume of voids over the total volume, as a percentage between 0 and 100% (or between 0 and 1). Determination of porosity is well known to a skilled artisan, e.g., using standardized techniques, such as mercury porosimetry and gas adsorption, e.g., nitrogen adsorption.

In some embodiments, pores can be of any desired pore size. As used herein, term “pore size” refers to a diameter or an effective diameter of the cross-sections of the pores. “Pore size” can also refer to an average diameter or an average effective diameter of the cross-sections of the pores, based on the measurements of a plurality of pores. In some embodiments, an effective diameter of a cross-section that is not circular equals the diameter of a circular cross-section that has the same cross-sectional area as that of the non-circular cross-section. In some embodiments, pores can have a size distribution ranging from about 50 nm to about 1000 μm, from about 250 nm to about 500 μm, from about 500 nm to about 250 μm, from about 1 μm to about 200 μm, from about 10 μm to about 150 μm, or from about 50 μm to about 100 μm. In some embodiments, stents can be swellable when hydrated. In some embodiments, sizes of pores can then change depending on the water content in the stent. In some embodiments, pores can be filled with a fluid such as water or air.

Methods for forming pores in silk fibroin-based scaffolds are known in the art and include, but are not limited, porogen-leaching methods, freeze-drying methods, and/or gas-forming method. Exemplary methods for forming pores in a silk fibroin-based material are described, for example, in U.S. Pat. App. Pub. Nos.: US 2010/0279112 and US 2010/0279112; U.S. Pat. No. 7,842,780; and WO2004062697, content of all of which is incorporated herein by reference in its entirety.

Though not meant to be bound by a theory, a stent's porosity, structure, and mechanical properties can be controlled via different post-spinning processes such as vapor annealing, heat treatment, alcohol treatment, air-drying, lyophilization and the like. Additionally, any desirable release rates, profiles or kinetics of a molecule encapsulated or embedded in the stent can be controlled by varying processing parameters, such as stent thickness, silk fibroin molecular weight, concentration of silk fibroin in the bulk material, beta-sheet conformation structures, silk fibroin II beta-sheet crystallinity, or porosity and pore sizes.

In some embodiments, stent designs are formed into a mold. In some embodiments, a mold is provided. In some embodiments, a mold design is machined into a surface of a mold. In some embodiments, a mold design is printed into a surface of a mold. In some embodiments, a mold design is formed using photolithography methods know in the art.

In some embodiments, designs are laser cut.

In some embodiments, radial openings are machine cut or laser cut from a cylindrical tube. In some embodiments, radial openings are r prefabricated with the stent.

### EXEMPLIFICATION

The following examples illustrate some embodiments and aspects of the disclosure. It will be apparent to those skilled in the relevant art that various modifications, additions, substitutions, and the like can be performed without altering the spirit or scope of the disclosure, and such modifications and variations are encompassed within the scope of the disclosure as defined in the claims which follow. The following examples do not in any way limit the disclosure.

### Example 1

The present Example describes a method of forming silk fibroin tracheal stents.

### Materials and Methods

In some embodiments, stents are comprised of an aqueous silk fibroin solution that may range in protein concentration from 1 to 50%, and may contain plasticizers or other additives. Specifically, a silk concentration of 15-30% results in a stent with the most advantageous mechanical properties for this application. Lower concentrations result in stents that shrink over time and are mechanically weak. Higher concentrations are challenging to process in a reproducible way.

In some embodiments, an average molecular weight of the silk can be controlled by the amount of time that it is degummed in a 0.02M Na₂CO₃ solution. The optimal molecular weight range for this application is achieved by degumming the silk fibers for 30 to 60 minutes. This corresponds to a number average molecular weight of approximately 50 to 150 kDa. Wray, L. S., Effect of Processing on Silk-based Biomaterials: Reproducibility and Bbiocompatibility, 99 Journal of Biomedical Materials Research Part B: Applied Biomaterials, 1, 89-101 (2011). Lower molecular weight silk solutions are easier to process into high concentrations, while higher molecular weight silk solutions result in less brittle final materials.

In some embodiments, stents are comprised of a soluble silk fibroin solution that is cast into a shape. Bulk silk fibroin solution is poured in flat smooth PDMS square molds measuring 8 inches per side. The solution is allowed to dry into films of application specific target thickness (150 to 300 μm) which is initially measured using a micrometer then verified using profilometry. Alternatively, solutions are lowered below the freezing point or dried under vacuum. Subsequently, solutions may go through an additional processing steps to induce or further induce beta sheet structure and reduce solubility.

In some embodiments, stents are excised in one piece from the silk films, using a laser to cut desired geometry, and then assembled using the micro ratchet and gear-racks. In some embodiments, ratcheted designs or designs with a larger radius are useful to accommodate nerves. Initial CAD designs are modified to be compatible with current equipment limitations, e.g., laser precision and feature loss due to heat affected zones. Alternatively, stent designs can be excised using a die cutting technique if heat affected zones become an issue.

In some embodiments, optimizing silk porosity and crystallinity in the processing step allows control over silk degradation. The freezing process imparts porosity into the material, which allows control over degradation rate as well as encourages cell migration into the stent. This is desirable as the stent degrades over time and is replaces by native tissue. Crystallinity may be augmented by post processing procedures, such as methanol treatment.

In some embodiments, temperatures of a solution are lowered to −45° C. at 1° C. per minute under ambient pressure for at least 12 hours, dried under vacuum at 50 mTorr for at least 24 hours, submerged in 90 wt% methanol for 1 to 3 hour, and then dried at ambient temperature and pressure. This gradual freezing cycle results in the optimal porosity and material homogeneity. Sudden drops in temperature may result in material heterogeneity. Methanol exposure induces beta sheet formation in the silk, rendering the final material insoluble and imparting strength. This is preferable over other procedures such as water annealing as it results in the strongest material.

In some embodiments, solutions were cast into the desired shape using a 180° Delrin mold. The described molding method is relatively scalable. Delrin has negligible porosity, is non-leachable, and is considered food-safe by the FDA. It also acts as an insulator during the freezing process. A material that is a better heat conductor may cause directional freezing of the stents, resulting in a non-homogenous material. The stent may range from 180-360° around and from 1/16 to ¼ inches in thickness. The size of the stent may be variable; an appropriate diameter can be chosen based on the patient's trachea size.

In some embodiments, formulations described may also be cast using a 3D printer. This can be achieved by printing onto an appropriately sized rod or half cylinder, or onto a printed support material. Further, the dimensions can be extracted from a computed tomography (CT) scan or other image in order to create patient specific stent geometry.

### Example 2

The present Example describes silk fibroin tracheal stents including a plasticizer.

In some embodiments, bulk material can include a silk fibroin:glycerol blend in a dry weight ratio of 75:25. The bulk material can be fabricated as described below. Other plasticizers, in addition to or instead of glycerol, can be used. Other weight ratios can also be used.

In some embodiments, bulk silk fibroin material can be formed from cocoons of the silk fibroin worm Bombyx mori (supplied by Tajima Shoji Co., Yokohama, Japan). Sodium carbonate, lithium bromide, and Slide-a-Lyzer dialysis cassettes can be purchased from Pierce, Inc. (Rockford, Ill., US). Silk fibroin solutions can be prepared by processing the silk fibroin cocoons. The B. mori silk fibroin cocoons can be boiled in 0.02 M aqueous Na₂CO₃ for 30 minutes to extract the sericin component and isolate the silk fibroin protein. The isolated silk fibroin can then be washed three times for 20 minutes in deionized water and allowed to dry for 48 hours at room temperature. The dried silk fibroin can be dissolved in 9.3 M LiBr at 60° C. for 4 h, and the resulting 20% (w/v) solution can be dialyzed against water using a Slide-a-Lyzer dialysis cassette (molecular weight cutoff 3500) for two days to remove salts. The resulting concentration of aqueous silk fibroin ranged from 5-7% (w/v), which was calculated by weighing the remaining solid after drying. The aqueous silk fibroin solution can be concentrated by exposing the cassette membrane to ambient air for varying times to produce a 10-20% (w/v) silk fibroin aqueous solution. Deionized water can be blended with the silk fibroin solutions to bring concentrations below 5%. The silk fibroin solutions can be stored at 4° C. until use. Aqueous fibroin solution prepared as described above can be used to cast sheet and tubular films as described above. The films can be fabricated by blending aqueous fibroin with 99% (w/v) glycerol to produce blends of 75:25 (dry weight) silk fibroin:glycerol solution.

### Example 3

The present Example describes silk fibroin stent designs.

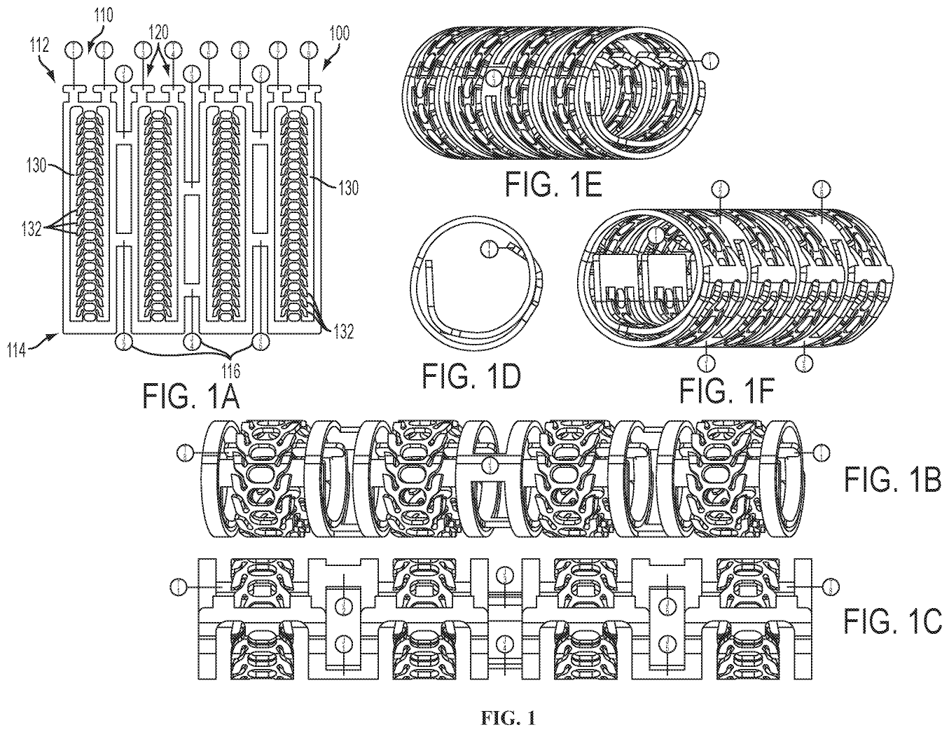

In some embodiments, silk fibroin tracheal stents can include one or more ratcheting mechanisms that allow the stent to be assembled into a small diameter configuration for insertion. FIGS. 1A-1F, 2A-2E, 3A-3C and 4A-4C provide illustrative examples of silk fibroin tracheal stents that include one or more ratcheting mechanisms according to some of the principles of the disclosure.

FIGS. 1A-1F show a ratcheting silk fibroin tracheal stent 100 according to a first embodiment of the disclosure. The stent 100 can include one or more stent elements 110, for example, FIGS. 1A-1F show four stent elements 110, each being connected or joint to one or more adjacent stent elements 110 by a joint 116. J denotes a joint, T denotes the tip of a strut gear rack, R denotes a ratchet slot. The joints 116 can be cut to enable the axial length of the stent 100 to be reduced. Each stent element 110 can include a first end 112 and a second end 114. The first end 112 can include one or more tabs 120 that are adapted to fit into slots 130. The slots 130 can extend from the second end 114 toward the first end 112 and while the FIGs shows the slot 130 extending all the way to the first end 112, the slot can end before reaching the first end 112, for example, ending in the middle of the device. The slot 130 can also include one or more teeth 132 that interact with tab 120 to control the diameter of the stent 100. The teeth 132 can be configured with an angled surface that allows the tab 120 to more easily slide past the teeth 132 in one direction (e.g. to increase in diameter) but resist movement in the opposite direction (e.g. to resist compressive forces that would reduce its diameter). The teeth 132 can be resilient or flexible to enable the tab 120 to more easily move past in one direction than in the opposite direction. The teeth 132 can extend at an angle with respect to the length of the slot 130 such that each tooth 132 flexes in one direction as the tab 120 moves past it but engages the end of the tooth 132 that resist movement of the tab 120 in the opposite direction.

In some embodiments, for example, FIGS. 1A-1F, each stent element 110 can be 5.0 mm wide by 30 mm long. When the stent 100 assembled into a tightly wound compressed tube, the stent 100 can have a minimum diameter of about 3.0 mm and can be expanded to a maximum diameter of about 10 mm. Each tab 120 can be “T” shaped and with the thinnest portion being approximately 1.5 mm wide. The thin portion of the tab 120 is adapted to fit in slot 130 which can be approximately 1.0 mm wide. The distance between each tab 120 and each slot 130 can be approximately 0.5 mm.

In some embodiments, tab 120 can be inserted into the slot 130 at a location close to the first end 112 with the second end curled around the inside of the stent 100 as shown in FIGS. 1B-1F. An outer sleeve can be used to hold the stent 100 in the compressed configuration for ease of insertion. After the stent 100 is inserted into the lumen of the vessel to be supported, the sleeve can be removed and an expansion force (e.g., such as that created by an expanding balloon) can be used to expand the stent 100 to the desired position. The number and location of the teeth 132 can be configured on regular intervals, for example, 100 micrometer increments, such that expanding the stent 100 by one tooth 132 increases the diameter by a predefined amount (in this example, 100/π micrometers).

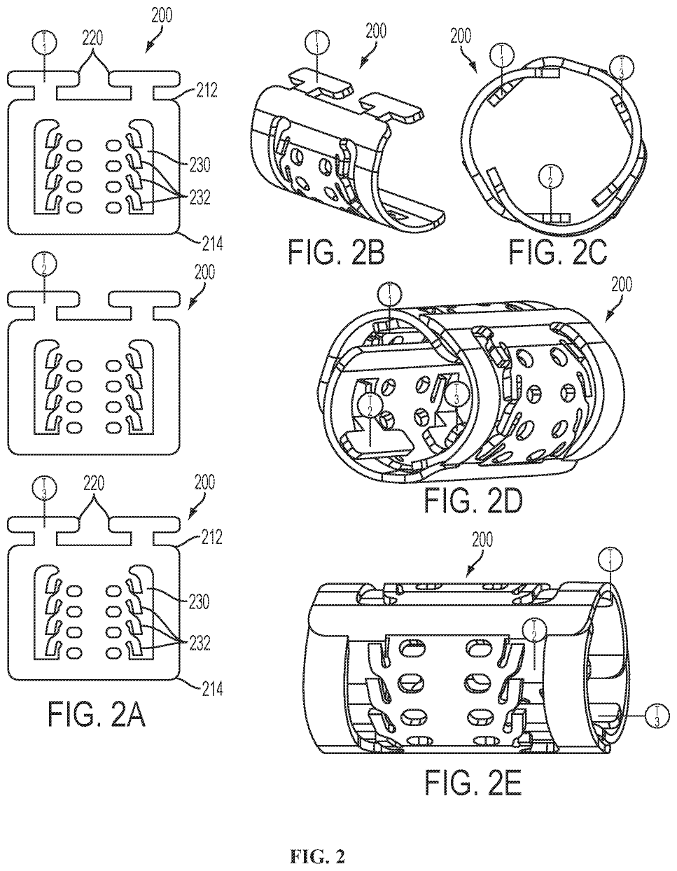

FIGS. 2A-2E show a ratcheting silk fibroin tracheal stent 200 according to a second embodiment of the disclosure. The stent 200 can be formed by combining two or more stent elements 210 in an end to end configuration as shown in FIGS. 2B-2E. While these figures show three, relatively short stent elements 210 connected end to end for form a hollow tube more stent elements 210 can be used to produce a larger diameter stent 200 and longer stent elements 210 can be used to enable larger variations in expanded stent diameter. Stent elements 210 of differing lengths can be used together. In this embodiment, each stent element 210 can be similar in the stent elements 110 shown in FIGS. 1A-1F. Thus each stent element 210 can include a first end 212 and a second end 214, the first end 212 can include one or more tabs 220 that are adapted to fit into slots 230. The slots 230 extend from the second end 214 toward the first end 212 and while the figure shows the slot 230 extending all the way to the first end 212, the slot can end before reaching the first end 212, for example, ending in the middle of the stent element 210. In this embodiment, the tabs 220 from one element can be inserted into the slots 230 of an adjacent stent element 210 to form circular chain of stent elements 210 with the second end 214 on the inside. The slot 230 can also include one or more teeth 232 that interact with tab 220 to control the diameter of the stent 200. The teeth 232 can be configured with an angled surface that allows the tab 220 to more easily slide past the teeth 232 in one direction (e.g. to increase in diameter) but resist movement in the opposite direction (e.g. to resist compressive forces that would reduce its diameter). The teeth 232 can be resilient or flexible to enable the tab 220 to more easily move past in one direction than in the opposite direction. The teeth 232 can extend at an angle with respect to the length of the slot 230 such that each tooth 232 flexes in one direction as the tab 220 moves past it but engages the end of the tooth 232 that resist movement of the tab 220 in the opposite direction. While FIGS. 2A-2E show only 1 row of stent element 210 axially, longer stents 200 can be created by widening each stent element 210 or by joining two or more stent elements 210 as shown in FIGS. 1A-1F.

In some embodiments, for example as shown in FIGS. 2A-2E, each stent element 210 can have substantially the same dimensions as the stent elements 110 shown in FIGS. 1A-1F, however the length of stent element 210 can range from 4 mm to 10 mm

In some embodiments, tab 220 can be inserted into the slot 230 at a location close to the first end 212 with the second end curled around the inside of the stent 200 as shown in FIGS. 2B-2E. An outer sleeve can be used to hold the stent 200 in the compressed configuration for ease of insertion. After the stent 200 is inserted into the lumen of the vessel to be supported, the sleeve can be removed and an expansion force (e.g., such as that created by an expanding balloon) can be used to expand the stent 200 to the desired position. The number and/or location of the teeth 232 can be configured on regular intervals, for example, 100 micrometer increments, such that expanding the stent 100 by one tooth 132 increases the diameter by a predefined amount (in this example, 100/π micrometers).

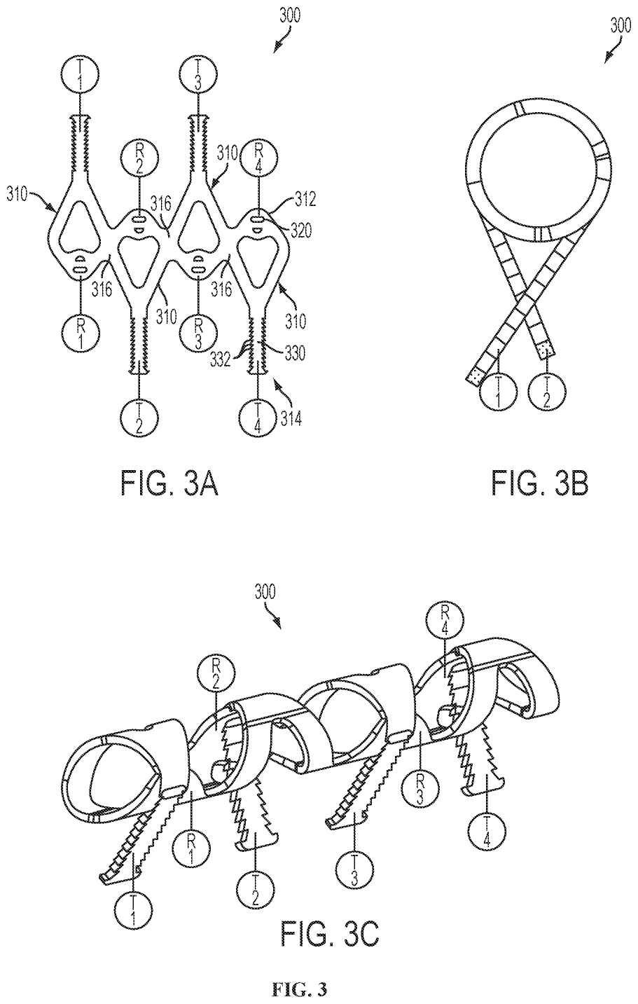

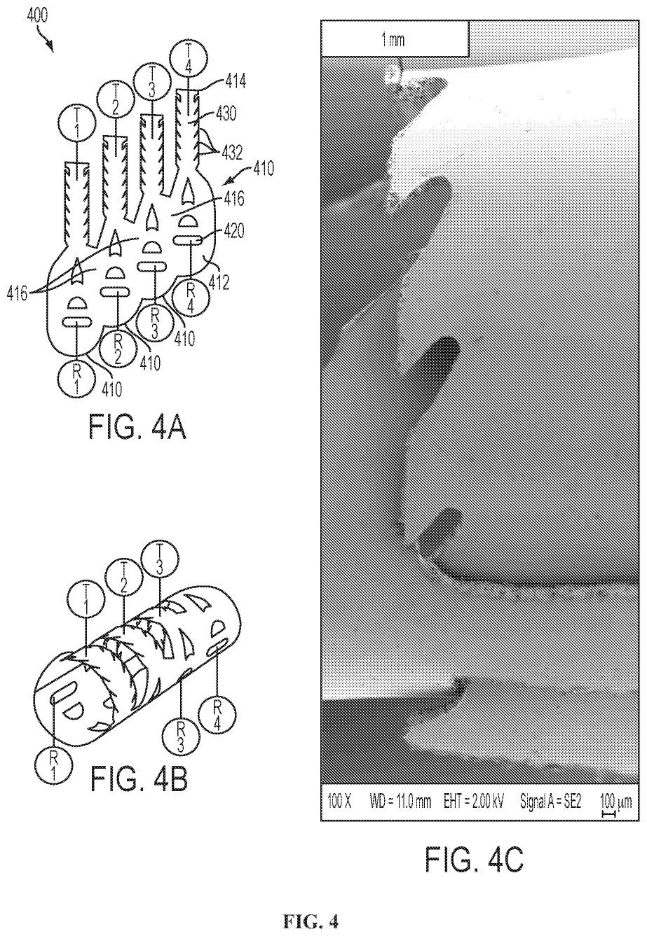

FIGS. 3A-3C show a ratcheting silk fibroin tracheal stent 300 according to a third embodiment of the disclosure. The stent 300 can include one or more stent elements 310, for example, FIGS. 3A and 3C show four stent elements 310, each being connected or joint to one or more adjacent stent elements 310 by a common side or joint 316. The joints 316 can be cut to enable the axial length of the stent 300 to be reduced. Each stent element 310 can include a first end 312 and a second end 314, and the first end 112 can include one or more tongues or strips 330 that are adapted to fit into slots 320. The strips 330 extend from first end 312 to the second end 314. The strip 330 can also include one or more teeth 332 that interact with slot 320 to control the diameter of the stent 300. The teeth 332 can be configured with an angled surface that allows the slot 320 to more easily slide past the teeth 332 in one direction (e.g. to increase in diameter) but resist movement in the opposite direction (e.g. to resist compressive forces that would reduce its diameter). The teeth 332 can be resilient or flexible to enable the slot 320 to more easily move past each tooth 332 in one direction than in the opposite direction. The teeth 332 can extend at an angle with respect to the length of the strip 330 such that each tooth 332 flexes in one direction as the slot 320 moves past it but engages the end of the tooth 332 that resist movement of the slot 320 in the opposite direction. In this embodiment, the strips 330 of adjacent stent elements 310 can extend in opposite direction, however in other embodiments, such as shown in FIGS. 4A and 4B, the strips can extend in the same direction.

FIGS. 4A-4C show a ratcheting silk fibroin tracheal stent 400 according to a fourth embodiment of the disclosure. The stent 400 can include one or more stent elements 410, for example, FIGS. 4A and 4C show four stent elements 410, each being connected or joint to one or more adjacent stent elements 410 by a common side or joint 416. The joints 416 can be cut to enable the axial length of the stent 400 to be reduced. Each stent element 410 can include a first end 412 and a second end 414, and the first end 412 can include one or more tongues or strips 430 that are adapted to fit into slots 420. The strips 430 extend from first end 412 to the second end 414. The strip 430 can also include one or more teeth 432 that interact with slot 420 to control the diameter of the stent 400. The teeth 432 can be configured with an angled surface that allows the slot 420 to more easily slide past the teeth 432 in one direction (e.g. to increase in diameter) but resist movement in the opposite direction (e.g. to resist compressive forces that would reduce its diameter). The teeth 432 can be resilient or flexible to enable the slot 420 to more easily move past each tooth 432 in one direction than in the opposite direction. The teeth 432 can extend at an angle with respect to the length of the strip 430 such that each tooth 432 flexes in one direction as the slot 420 moves past it but engages the end of the tooth 432 that resist movement of the slot 420 in the opposite direction. In this embodiment, the strips 430 of adjacent stent elements 410 can extend in the same direction, however in other embodiments, such as shown in FIGS. 3A and 3B, the strips 430 of adjacent stent elements 410 can extend in the same direction. In some embodiments of the disclosure, the stent elements 410 can be offset in a direction transverse to the cylindrical axis of the stent 400 (e.g., around the circumference of the tubular stent). FIG. 4C shows SEM magnification of stent demonstrating 1-way slotted joint.

In some embodiments, silk fibroin tracheal stent design can employ micro ratchet slot and gear-rack mechanisms to enable large increases in diameter. Additionally, this design allows for deployment times within standard clinical limits and can be deployed faster than the 30-60 second requirement of current metal stents due to the lack of radial recoil associated with metal deformation. In some embodiments, provided stents may be deployed in less than 60 seconds, less than 50 seconds, less than 40 seconds, less than 30 seconds, less than 20 seconds, or less than 10 seconds. Fine tuning of the bulk material properties independently of the tab and slot geometry, which also enable the assembly, allows an additional level of control of the resorbable implant for extended periods of time. By utilizing the design to incorporate sophisticated yet simple mechanically articulating strut and joint assemblies, the initial flexibility and compliance of one piece material constructs can be tuned to control the bend flexibility while maintaining radial strength. The stent mechanical design can be optimized to meet desired functional requirements: (a) the stent can be deployed using standard clinical deployment tools, (b) the stent can be deployed with a low risk of injury by not requiring over-dilation or extended dilation times, (c) the stent can provide a predefined amount of radial strength because the radial strength is dependent on the assembled slot and tab which can be discretely measured at each position.

In some embodiments, ratcheting stent designs (e.g., stents 100, 200, 300, 400) can be formed from a sheet of silk fibroin material, such as a silk fibroin or blend of silk fibroin with other materials that can enhance the properties of the silk fibroin for specific applications. The sheet of silk fibroin material can be cast (e.g., in PDMS molds) to produce a predefined thickness according to the application, for example, in the range of 100 micrometers to 500 micrometers, in the range of 150 micrometers to 450 micrometers, in the range from 200 micrometers to 400 micrometers, in the range from 250 micrometers to 350 micrometers. Where increased strength is desired, thicknesses above 300 micrometers can be used. Where faster resorbability is desired, thickness below 300 micrometers can be used. According to various embodiments, it may be desirable for thickness to be 300 micrometers or less. In accordance with some embodiments of the disclosure, the sheet material of a specific thickness (e.g., 150 to 300 micrometers) can die cut, micro-machined or laser cut into the desired configuration. In accordance with other embodiments of the disclosure, the silk fibroin material can be cast directly into the desired configuration. The silk fibroin tracheal stents in flat form can be distributed to healthcare providers for assembly and insertion as needed. After the silk fibroin tracheal stent is formed, the flexible material can be assembled by hand or machine into small diameter tubes, using the ratcheting mechanism (and in some embodiments, assisted by an outer sheath) to hold the stent in its small diameter form prior to insertion. After insertion, any outer sheath can be removed and the silk fibroin tracheal stent can be expanded in place to provide the desired internal diameter for fluid flow and structural support. The axial length of the stent can be selected according to the application and the needs of the patient to be treated. In accordance with some embodiments of the disclosure, the axial length of the ratcheting stent can be in the range from 1 millimeter to 50 millimeters, in the range from 10 mm to 40 mm, in the range from 20 mm to 30 mm. In some embodiments, ratcheting stent can be formed from a long sheet that provides 2 or more stent segments that can be separated to provide a silk fibroin tracheal stent of a predefined length.

In some embodiments, a ratchet stent design employs micro ratchet slot and gear-rack mechanisms to enable large increases in diameter. Additionally, this design allows deployment times within standard clinical limits and is faster than the 30-60 second requirement of current metal stents due to the lack of radial recoil associated with metal deformation. Fine tuning of the bulk material properties independently of these engineered joints, which also enable the assembly, allows an additional level of control of the resorbable implant for extended periods of time. By utilizing the design to incorporate sophisticated yet simple mechanically articulating strut and joint assemblies, the initial flexibility and compliance of one piece material constructs can be tuned to control the bend flexibility while maintaining radial strength. The stent mechanical design is optimized to meet certain conditions: (a) The joints are engineered to provide as much mechanical function as possible without adding significant protrusions to the implant, (b) the stent is deployable using standard clinical deployment tools, (c) the stent minimizes deployment injury by not requiring over-dilation or extended dilation times, (d) the stent provides sufficient radial strength.

### Example 4

The present Example describes flexible silk fibroin tracheal stents.

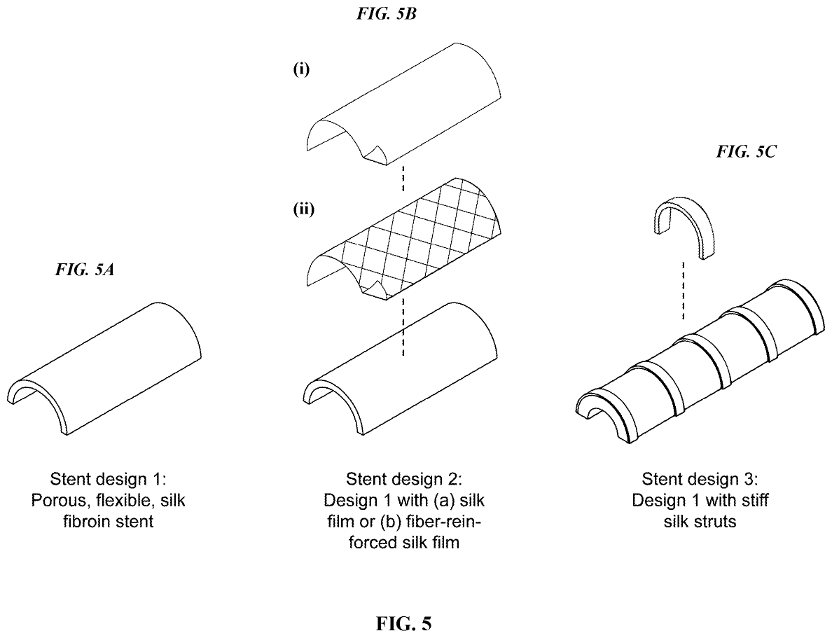

In some embodiments, a silk fibroin tracheal stent does not include an additional layer of support or an addition supporting mechanism. FIG. 5 at panel 5A shows a porous, flexible silk fibroin tracheal stent.

In some embodiments, a bare silk fibroin tracheal stent includes 5-30% silk fibroin w/w. A mold is provided. A mold is either flat or includes features that are machine or laser cut. A silk fibroin solution is poured into a mold.

The resultant bare silk fibroin tracheal stent is porous and flexible.

A bare silk fibroin tracheal stent is substantially cylindrical. A substantially cylindrical bare silk fibroin tracheal stent may have a radial opening between of about 180°+/−60°. Openings are molded, machined, cut, or laser cut.

### Example 5

The present Example describes reinforced silk fibroin tracheal stents.

In some embodiments, silk fibroin tracheal stents are reinforced with silk films. As such, a silk film is added to a silk fibroin tracheal stent.

FIG. 5 at panel FIG. 5B shows films added or fused to a silk fibroin tracheal stent as provided herein.

FIG. 5 at panel FIG. 5B at (i) shows a solid film of silk. A thickness of such a silk film is adjustable.

In some embodiments, silk fibroin tracheal stents are reinforced with polymer films. As such, a polymer film is added to a silk fibroin tracheal stent.

In some embodiments, silk fibroin tracheal stents are reinforced with films. Films may also be reinforced with silk fibers. As such, a silk fiber reinforced film is added to a silk fibroin tracheal stent. Films may also be reinforced with metal. As such, a metal reinforced film is added to a silk fibroin tracheal stent.

FIG. 5 at panel FIG. 5B at (ii) shows a reinforced film of silk and silk fiber. A thickness of such a silk film is adjustable.

In some embodiments, silk fibroin tracheal stents are reinforced with stiff silk films. As such, a stiff silk film is added to a silk fibroin tracheal stent.

In some embodiments, an additional film layer is fused to a top of a silk fibroin tracheal stent. As such, an added or fused layer is designed and engineered to meet a desired biodegradation profile.

FIG. 5 at panel FIG. 5B shows orientation for addition or fusion of a reinforcement layer, of (i) silk film and/or (ii) silk film that is fiber reinforced.

In some embodiments, for example, when an additional or fused layer is silk fibroin, such silk fibroin films include a silk fibroin solution of about 10-40% w/w.

### Example 6

The present Example describes silk fibroin tracheal stents reinforced with struts.

In some embodiments, stiff silk struts are added or fused to a silk fibroin tracheal stent backbone. As such, struts are radially fused to a silk fibroin tracheal stent backbone.

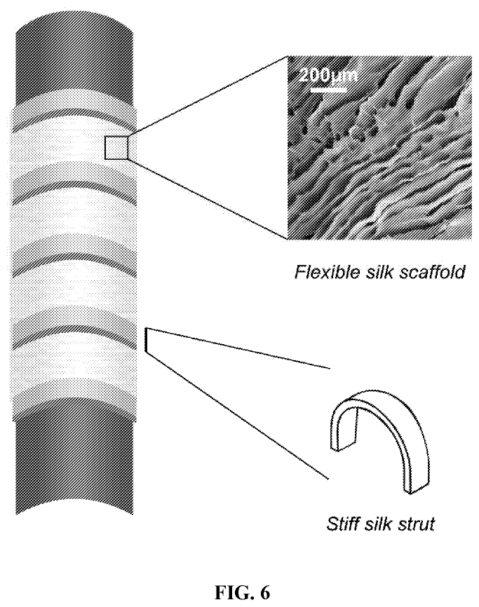

FIG. 5 at panel FIG. 5C and FIG. 6 shows stiff silk struts spaced and fused to a flexible silk fibroin tracheal stent. The exploded region of FIG. 6 shows a flexible silk fibroin scaffold as a body of the silk fibroin tracheal stent and stiff silk fibroin for struts spaced and fused thereto.

In some embodiments, struts are silk fibroin. In some embodiments, silk fibroin struts are about 20-40% w/w silk.

Silk fibroin struts are produced by any means known in the art, including 3D printing of a dissolvable support material or by machining from a block of silk.

In some embodiments, struts added or fused to a silk fibroin tracheal stent can be modified to control radial strength, such that inward or outward force of can be applied or supplemented with strut to a silk fibroin stent.

In some embodiments, struts added or fused to a silk fibroin tracheal stent can be modified to control strut width or thickness.

In some embodiments, a number of struts and/or spacing of struts as applied to a silk fibroin tracheal stent can be adjusted.

### Example 7

The present Example describes in vivo studies of silk fibroin tracheal stents as disclosed.

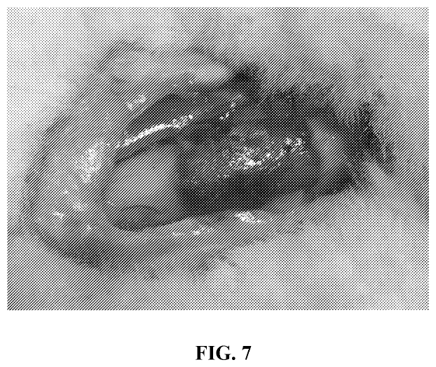

FIG. 7 shows a flexible silk fibroin stent implanted in preclinical rabbit model for 3 months. Reinforced silk fibroin stents were also implanted in preclinical rabbit model for 3 months.

Rabbits survived for the duration of the 3 month study. Rabbits exhibited normal behavior according to animal care technicians. Stents were cut to size. Stents were easy to suture to the rabbits and exhibited no stent migration.

### Example 8

The present Example describes silk fibroin tracheal stents made by 3D printing.

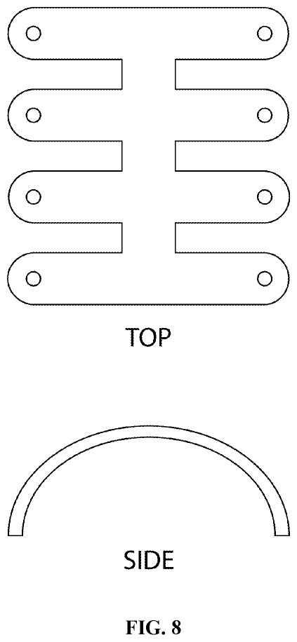

In some embodiments, provided tracheal stents are fully printable using a 3D printer. FIG. 8 shows a side view and a top view of a tracheal stent design that is printed with a 3D printer. In some embodiments, silk fibroin struts are 3D printed from a silk fibroin solution having about 20-40% w/w silk.

### Example 9

The present Example describes deployment of silk fibroin tracheal stents as disclosed.

In accordance with embodiments of the present disclosure, the stents can be deployed using standard surgical techniques.

In accordance with embodiments of the present disclosure, the stents can be deployed using standard balloon catheters using familiar surgical procedures, in particular wire guidance and balloon inflation. Prior art natural silk fibroin tracheal stents are traditionally incompatible with balloon deployment devices due to the difference in ductility and plastic deformability compared to metal counterparts. The stents according to the disclosure can be deployed using standard plastic and silicone deployment devices currently clinically used. In accordance with some embodiments of the disclosure, the different stent designs can make use of a different mechanism when mounting the stent onto the catheter, and subsequently a different mechanism of dilation during deployment. This does not impact the surgeon's standard procedures at the time of deployment.

In accordance with some embodiments of the disclosure, a one piece solid mesh structure can also be used. This mesh structure design can be deployed using the same balloon deformation mechanism of current metal stenting technology. However, this embodiment may not be able to provide the same amount of radial support as some of the other embodiments described herein.

The ratcheting stent according to some embodiments of the disclosure provides a one-way mechanical mechanism for discrete expansion, using, for example, a slot and gear or tooth that can function similar to a zip-tie or ratcheting tie wrap. The stent body can contain a slotted portion while the stent struts or strips can include a gear or tooth rack. To avoid potential blood flow obstruction caused by a traditional square ratchet head, the design can be modified to utilize two parallel slots and to use parallel slots with double sided gear racks.

In accordance with some aspects of the disclosure, the stent body can be fabricated in one piece by excising the body from film sheets using a laser. In some embodiments, the silk fibroin sheets can suffer from significant burn zones near the cut edge which blunts and recedes the edge from the intended cut, and rounds reciprocated cuts. To compensate for and avoid theses defects, the minimum feature size in those burn zones can be enlarged in order to maintain the functionality provided by the ratchet mechanism. Additionally, stent functionality and uniformity of mechanical strength can be improved by increasing the number of tabs and slots, and distributing them symmetrically within the device.

In accordance with some embodiments of the disclosure, glycerol can be added to the silk fibroin film blend to improve solubility and stent surface compliance, flexibility and resilience. The addition of glycerol can also improve the fabrication process by reducing the defects in the burn zones and sharpening the features of the device.

Uniaxial compressive resistance of initial stent revisions can be highly dependent on the angle relative to the ratchet slot. Further revisions of the stent design can incorporate modifications to the position of the slots and tabs, for example, distributing them symmetrically within the device such that, when assembled, the stent appeared to have radial symmetry, which can make compressive resistance more uniform.

Stent deployment was initially met with difficulty. The ratchet slot design enabled one-way expansion and successfully prevented reverse sliding, but in many balloon dilation trials within silicone tubing resulted in tearing of the stent material rather than smooth expansion. Several revisions were designed to facilitate sliding of the struts without compromising the one-way function. The geometry of the teeth can be changed such that they bent easier in one direction. Rounded tips and additional filleting can also be added to the teeth.

The resistance of the stent to remain folded exerts a force on the inside surface of the sheath which produces enough friction to cause the stent to move away from the target site during retraction of the sheath. A seat at the base of the balloon and attached to the catheter can be used to limit sliding of the stent during sheath retraction.

The tubular silk fibroin material is very strong and the addition of glycerol to the film blend can improve compliance, flexibility and resilience. The formulation of the silk fibroin:glycerol blend can be used to impart plasticizing properties which can avoid creasing of the stent at the points of folding.

In accordance with some embodiments of the disclosure, the silk fibroin tracheal stent can be formed of a material that has an average mechanical stiffness in the range from 0.5 kN/m to 3.0 kN/m, in the range from 1.0 kN/m to 3.0 kN/m, in the range from 1.0 kN/m to 2.5 kN/m, in the range from 1.0 kN/m to 2.0 kN/m, in the range from 1.0 kN/m to 1.5 kN/m, and other stiffness ranges can be used. In one embodiment of the disclosure, the stent can be include a cylindrical body having an average mechanical stiffness of approximately 1.2 kN/m. In accordance with some embodiments of the disclosure, the silk fibroin tracheal stent can be formed of a material that has an average radial strength in the range from 100 mmHg to 500 mmHG, in the range from 100 mmHg to 400 mmHg, in the range from 200 mmHg to 400 mmHg, in the range from 200 mmHg to 300 mmHg, in the range from 250 mmHg to 350 mmHg, and other stiffness ranges can be used. In one embodiment of the disclosure, the stent can be include a cylindrical body having an average radial strength of approximately 300 mmHg.

References cited in the present disclosure are hereby incorporated by reference in their entirety. Other embodiments are within the scope and spirit of the disclosure. Features implementing functions may also be physically located at various positions, including being distributed such that portions of functions are implemented at different physical locations.

Further, while the description above refers to the disclosure, the description may include more than one invention.

### Example 10

The present Example describes deployment of silk fibroin tracheal stents as disclosed.

In accordance with embodiments of the present disclosure, silk fibroin tracheal stents can be deployed to assess the safety and efficacy of a silk fibroin-based splint in a clinically relevant model of tracheomalacia, and to provide quantitative clinical outcomes.

In accordance with embodiments of the present disclosure, the silk fibroin tracheal stents were evaluated in a surgically induced model of severe tracheomalacia in N=3 New Zealand white rabbits for durations of 17, 24, and 31 days. A dynamic change in airway diameter during spontaneous respiration was measured in the native trachea (control), after surgical intervention, after stent placement, and at the time of explant.

In accordance with embodiments of the present disclosure, a study of a silk fibroin-based external tracheal splint for the treatment of severe tracheal collapse in pediatric patients was conducted. Safety and efficacy of such silk fibroin-based external tracheal splints were assessed in a clinically relevant model of severe tracheomalacia. Dynamic airway collapse was quantified by a newly established imaging method 1) before injury, 2) after injury, 3) after splint placement, and 4) at the time of explant, and the surrounding tissue response was assessed by histology analysis. This study reveals a promising opportunity for external resorbable splints fabricated from silk fibroin for the treatment of severe tracheomalacia.

### Materials and Methods

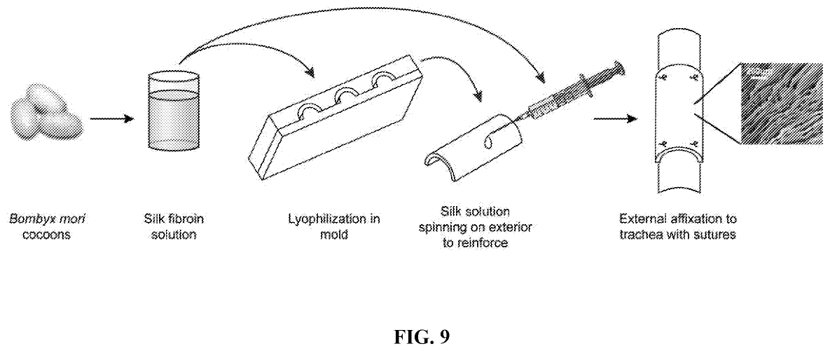

Splints were fabricated from reconstituted silk fibroin in a two-step process shown in FIG. 9. First, 150 mg/mL aqueous silk fibroin purified from native Bombyx mori cocoons, was cast into molds and lyophilized to form solid 180° splint constructs (freezing: 0.5° C./min to 40° C., primary drying: −20° C. at 100 mTorr, secondary drying: 4° C. at 100 mTorr). The splints were then immersed in methanol for 1 hour to render them insoluble. Next, 150 mg/mL silk fibroin was spin cast onto the exterior of the splint to a thickness of approximately 1 mm, and the coated splints were immersed in methanol for 60 min. Silk fibroin splints were sterilized with ethylene oxide and allowed to off-gas for at least 72 hours.

Mechanical properties were assessed with a uniaxial mechanical tester (model 3366, Instron Inc.) as the splints were degraded in a protease solution (1.0 U/mL Protease XIV in phosphate buffered saline (PBS), Sigma-Aldrich) at 37° C. to mimic in vivo degradation. Mechanical testing of the hydrated splints comprised of a cyclic compression test (0 to 30%) with 5 cycles at a rate of 1.0 mm/min; the maximum force was extracted from the peak force in the final cycle. (N=4 independent samples per time point). Degradation was determined by the change in mass of the dried sample from the Day 0 dry mass. A control group of N=4 was stored in PBS. Samples were imaged under scanning electron microscopy (SEM) at Days 0 and 43 to observe the surface topography as they degraded.

### Tracheomalacia Model and Study Design

We sought to validate the external tracheal splint design in a New Zealand white rabbit model in a small study. This work was approved by the Massachusetts Eye and Ear Institute (MEET) Institutional Animal Care and Use Committee (IACUC) (protocol #15-006). We selected the rabbit as a model for its tracheal anatomy and intrinsic dynamics, which are comparable to those of the human infant. Three male rabbits (weights approximately 3 kg) were used in this study, with the male gender selected for the relative paucity of redundant skin overlying the neck (“dewlap”) compared to females. Pre-anesthetic sedation and analgesia was achieved by the intramuscular administration of ketamine (25 mg/kg) and xylazine (3.75 mg/kg). Once sedated, the ear was prepped and a fentanyl (12 mcg/hr) transdermal patch was applied for post-operative analgesia. Then, a catheter was placed in the auricular vein and buprenorphine (0.05 mg/kg) was given intravenously (IV) once. Maintenance IV fluids were initiated and the rabbit was placed supine on a water-circulating warming pad. Pulse oximetry was applied and blood oxygen saturation (SpO₂) and heart rate was monitored continuously throughout the procedure. A laryngeal mask airway (LMA) was inserted, and position checked by “fogging” of the tube with respirations and observation of symmetric chest rise. Anesthesia was then induced with isoflurane (1-5%), and supplemental oxygen was bled in to the ventilator circuit and titrated as necessary to maintain an SpO₂>92%. End-tidal CO₂ (EtCO₂) monitoring was continuous throughout the procedure.

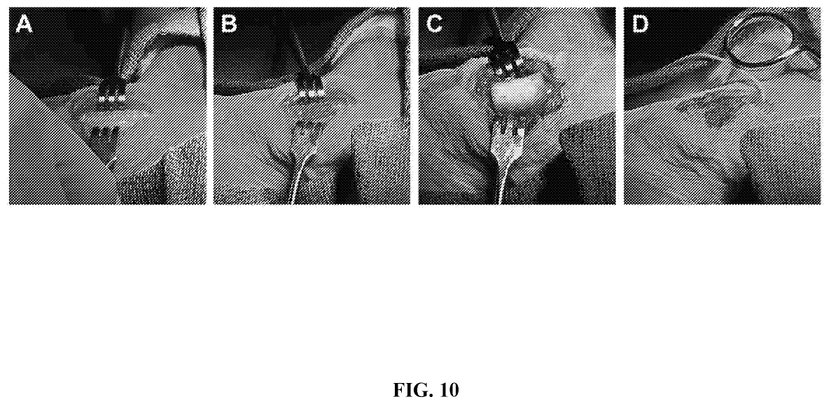

The neck was then prepped by removing overlying fur and sterilizing with betadine swabs. A flexible bronchoscope (Karl Storz 11101rp2 flexible rhino-pharyngo laryngoscope, OD 3.5mm) was then passed into the ventilator circuit via a port, then through the LMA and past the vocal folds into the trachea. The position of the bronchoscope was noted by observing the depth markings on the scope sleeve, and the light at the distal tip of the bronchoscope through the overlying soft tissue of the neck. Video of at least three spontaneous breath cycles, defined as a full inspiration and expiration, was recorded for subsequent airway dynamics assay described below, and then the bronchoscope was withdrawn. A cervical incision was made over the airway extending superiorly from the level of the cricoid cartilage and inferiorly over the upper cervical trachea. The incision was carried down through the skin and subcutaneous tissues to the level of the strap musculature, which were divided in the midline and retracted laterally to expose the underlying trachea. The surgical technique for inducing tracheomalacia was developed by Dr. Kishore Sandu (oral communication, 2016). FIG. 10 shows using a Beaver® mini-blade, the anterior aspect of four tracheal rings overlying the site of the initial airway dynamics assay were meticulously dissected away from the underlying tracheal mucosa to approximately the point of the trachea-esophageal grove. At this time, the rabbit was given positive pressure breaths via the ventilator circuit to check for air leak. Uniformly, at this step in the protocol no air leak was observed for the three rabbits.

The bronchoscope was then inserted to the same depth using the printed ruler on the sheath, and a video recording of at least three spontaneous breath cycles was again made. The bronchoscope was withdrawn, and the bioresorbable tracheal splint, previously hydrated in sterile PBS, was overlain on the denuded area of trachea. Shown in FIG. 10, the splint was sutured to the tracheal mucosa at the lateral aspects of the splint with Monocryl suture. The overlying musculature was then re-approximated with 3-0 Vicryl suture. The skin was closed in a multi-layer fashion with 3-0 Vicryl buried deep dermal sutures and running 5-0 fast absorbing plain gut. The rabbit was again given positive-pressure breaths to observe for air leak, which commonly occurred following placement of the sutures in the tracheal mucosa. The air, however, easily escaped via the rubber band drain following cessation of positive-pressure ventilation. Following full closure of the surgical incision the bronchoscope was again placed through the LMA to the pre-determined depth, a third video of three spontaneous breath cycles was recorded, and the scope was withdrawn.

The rabbit was recovered in the operating room, and returned to the animal care facility (ACF) when righting reflex reemerged. Each rabbit received twice-daily subcutaneous boluses of normal saline for the first 72 hours post-operatively, and was otherwise observed and cared for per protocol in the ACF until the end of the study period. At either post-operative day 17, 24, or 31, respectively, each rabbit was brought back to the operating room. Pre-operative sedation, analgesia and operative anesthesia were achieved through the use of ketamine, xylazine, and isoflurane as above. The rabbit was allowed to breathe spontaneously on a ventilator circuit via LMA. A bronchoscope was passed through the LMA to the level of the depth markings noted at the time of splint placement with additional confirmation by palpation of the splint and observation of the airway movement on the bronchoscope. A video recording of three breath cycles was obtained. Then, the rabbit was euthanized using Fatal-PlusTM solution (pentobarbital sodium, active ingredient), with completed euthanasia confirmed via pulse oximetry and cardiac auscultation. The neck was then incised and the trachea was then resected en bloc with the splint in place, and placed in paraformaldehyde (PFA, 4% in PBS) for fixation. This tissue was then processed in paraffin section and stained with hematoxylin and eosin. Photomicrosopy of relevant aspects was obtained and is presented below.

Pre-Clinical Outcomes

Following tracheal splint implantation, the rabbits were observed and maintained per ACF guidelines, and monitored for respiratory distress, stridor, weight loss, and other objective signs of post-operative complications.

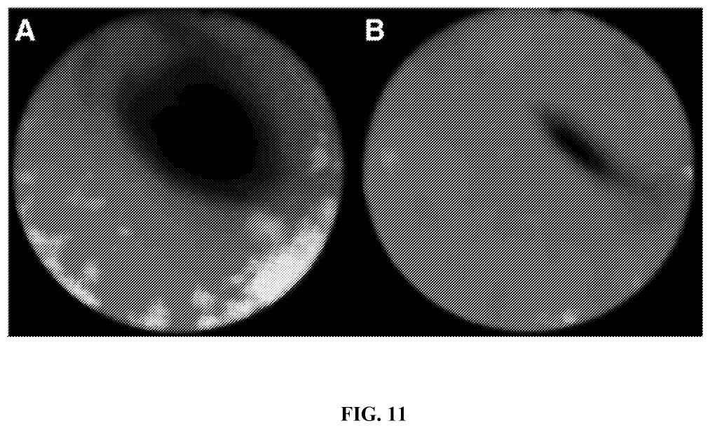

An image-based assay was used to quantify the degree of tracheomalacia in our model. FIG. 11 shows a still image from each bronchoscopic video recording, still images of the maximum and minimum airway lumen were generated for each of three recorded breath cycles. These still images were then optimized to maximize contrast between the lumen and tracheal wall. The resultant optimized images were then randomized and provided to a blinded rater. The area of the airway lumen was determined by tracing the wall of the lumen in ImageJ, which outputs an area in pixels. Using these measurements, percent tracheal collapse was calculated in triplicate for each rabbit at each operative stage using equation (1).

[(Lumenₘₐₓ−Lumenₘᵢₙ)÷Lumenₘₐₓ]×100=% Tracheal Collapse (1)

FIG. 11 shows an example of the surgically-induced tracheomalacia in a rabbit airway prior to implantation of the bioresorbable silk fibroin splint. A, maximal lumen size with tidal expiration, B, minimum lumen size during spontaneous inhalation. Using the described quantitative image-based assay, this example represents a calculated 93% reduction in airway caliber.

Paired images were acquired for three breath cycles for each animal in the normal trachea, immediately following surgical inducement of tracheomalacia, again after splint implantation, and then at either post-operative day 17, 24, or 31.

Fabrication and Mechanics

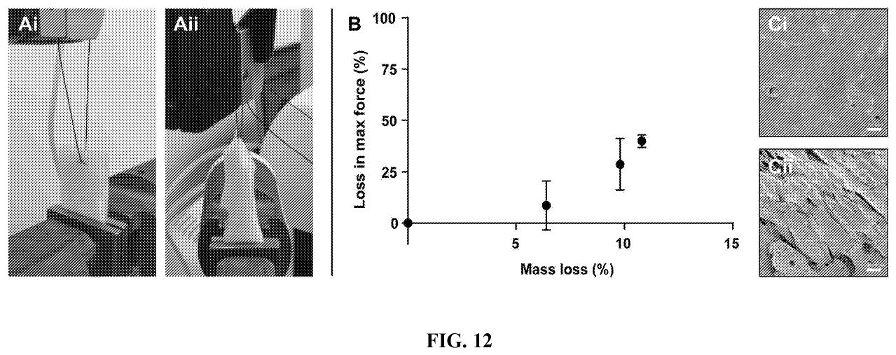

The two-step fabrication process comprising of freeze drying and spin coating resulted in silk fibroin splints that were both flexible and stiff. As shown in FIG. 12 at panel A, freeze drying yielded a porous and flexible splint that could be contoured to fit the external tracheal wall, cut to size on the operating table if needed, and hold a suture passed through anywhere on the splint, thus eliminating the need to use to preformed holes. Spin coating the external surface of the splint with concentrated silk fibroin solution allowed us to control the stiffness of the device. FIG. 12 at panel B shows the silk fibroin splints exhibited a loss in the maximum force (measured by cyclic uniaxial compression testing) as they degraded in protease solution at 37° C. in a linear fashion. A mass loss of 10% correlated to a loss in maximum force of approximately 30%. FIG. 12 at panel C shows evidence of degradation could also be observed in SEM images of the silk fibroin splints at 0 (top panel) and 10.8% mass loss (bottom panel).

Rabbit Survival and Outcomes

The rabbits uniformly tolerated the tracheal splint implantation and immediate post-operative period well. Subcutaneous air collection was minimal, and the rubber band drain remained functional and in place for 24-48 hours. In all cases, the rabbit displaced or removed the rubber band prior to planned removal with cessation of subcutaneous air collection. Some of the animals had modest biphasic stridor noted with agitation (e.g. during toenail trimming or other semi-invasive routine ACF care. When stridor was present, the animals continued to breath comfortably without respiratory distress. At all other times, the rabbits had quiet unlabored breathing throughout their recovery. Wound healing was uncomplicated in all cases. None had weight loss, nor any other signs of distress or failure to thrive during their approximately 2-4 weeks of recovery.

Airway Measurements and Tracheal Histology

An image based assay was developed to quantify the change in dynamic airway diameter, providing a metric to assess both the severity of the surgically induced malacia as well as the efficacy of the device. Histopathology was used to study the splint degradation and the surrounding tissue response.

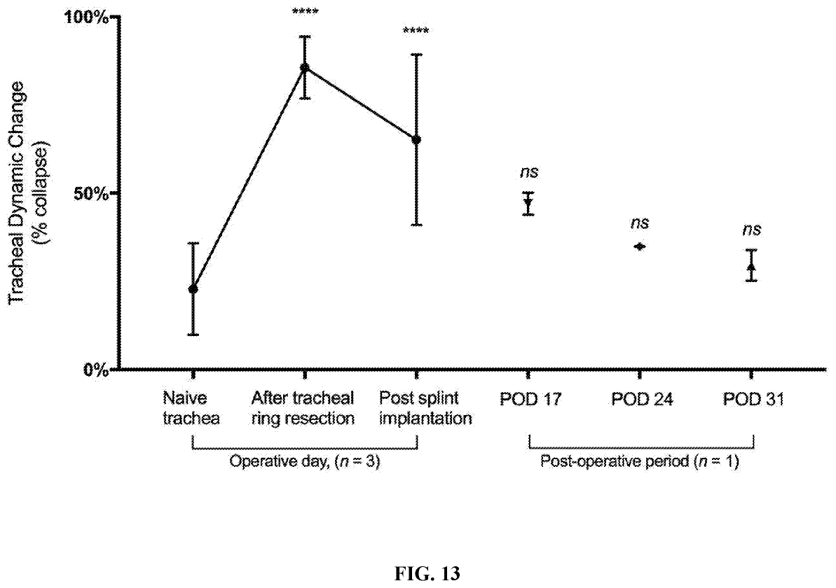

The average intrinsic dynamic change of the naive rabbit trachea was 22.8% with spontaneous tidal respirations. Surgical resection of the anterior tracheal rings was effective at inducing tracheal collapse, with a significant increase in average change in airway caliber to 85.6% (p <0.001). Following splint implantation, there was a decrease in average collapsibility to 65.2%, which remained significantly increased relative to naive dynamics (p<0.001). The first rabbit was explanted at 17 days post-implantation. At that time, tracheal dynamic change with spontaneous respirations was further decreased to 47%, which was not significantly different than naïve trachea (p=0.18). This trend continued with the second rabbit, explanted at 24 days with a tracheal dynamic change of 34.9% (p=0.83); and the third rabbit, explanted at 31 days with a tracheal dynamic change of 29.6% (p=0.98). Representative data is shown in Table 3 and FIG. 13.

Tracheal dynamic change as measured using an image-based assay. Surgically-induced tracheomalacia resulted in a significant increase in dynamic change in airway caliber with tidal respirations. Subsequent splint placement reduced tracheal dynamic change to a level statistically equivalent to the naive trachea by post-operative day 17, and this persisted through post-operative day 31.

TABLE 3
Airway lumen measurements

| | Breath | | | | |
|---|---|---|---|---|---|
| VIDEO | Cycle | Max | Min | % Change | ×100 |
| C59 - Naïve | A | 6078 | 3657 | 0.39832182 | 39.8321816 |
| | B | 6956 | 4245 | 0.38973548 | 38.973548 |
| | C | 6389 | 3913 | 0.38754109 | 38.7541086 |
| C59 - Post-resection | A | 6139 | 367 | 0.94021828 | 94.0218277 |
| | B | 4931 | 178 | 0.96390185 | 90.3901845 |
| | C | 6866 | 316 | 0.95397611 | 95.3970114 |
| C59 - Splinted | A | 1141 | 45 | 0.96056091 | 96.0560911 |
| | B | 1199 | 91 | 0.92410342 | 92.410342 |
| | C | 1272 | 59 | 0.95361635 | 95.3616352 |
| C59 - Explant | A | 6022 | 2975 | 0.50597808 | 50.597808 |
| | B | 5749 | 3135 | 0.45468777 | 45.4687772 |
| | C | 5688 | 3127 | 0.45024613 | 45.0246132 |
| Rabbit 29 - Naïve | A | 1122 | 871 | 0.22370766 | 22.3707665 |
| | B | 1245 | 1029 | 0.17349398 | 17.3493976 |
| | C | 1057 | 929 | 0.12109745 | 12.1097446 |
| Rabbit 29 - Post- | A | 4525 | 556 | 0.87712707 | 87.7127072 |
| resection | B | 5252 | 724 | 0.86214775 | 86.2147753 |
| | C | 4427 | 658 | 0.85136661 | 85.1366614 |
| Rabbit 29 - Splinted | A | 3717 | 2450 | 0.34086629 | 34.086629 |
| | B | 3865 | 2216 | 0.42664942 | 42.6649418 |
| | C | 4075 | 2341 | 0.42552147 | 42.5521472 |
| Rabbit 29 - Explant | A | 5531 | 3600 | 0.34912312 | 34.9123124 |
| | B | 5589 | 3635 | 0.34961532 | 34.9615316 |
| | C | 5863 | 3818 | 0.34879754 | 34.8797544 |
| Rabbit C58 - Naïve | A | 4809 | 4306 | 0.10459555 | 10.459555 |
| | B | 4424 | 4061 | 0.08205244 | 8.20524412 |
| | C | 4672 | 3858 | 0.17422945 | 17.4229452 |
| Rabbit C58 - Post- | A | 6905 | 1769 | 0.74380883 | 74.3808834 |
| resection | B | 7494 | 1719 | 0.77061649 | 77.0616493 |
| | C | 7169 | 1835 | 0.74403683 | 74.4036825 |
| Rabbit C58 - | A | 2876 | 1038 | 0.63908206 | 63.9082058 |
| Splinted | B | 2514 | 937 | 0.62728719 | 62.7287192 |
| | C | 2831 | 1225 | 0.56729071 | 56.729071 |
| Rabbit C58 - | A | 4281 | 3062 | 0.28474655 | 28.4746555 |
| Explant | B | 4409 | 3270 | 0.25833522 | 25.8335223 |
| | C | 4488 | 2946 | 0.34358289 | 34.3582888 |

Histopathology demonstrated a localized inflammatory process at the interface of the bioresorbable splint and tracheal mucosa, including apparent infiltration of fibroblasts into the proximal aspect of the silk matrix. Degradation of the SF splint was not observed, and is not expected to occur within the time period studied.

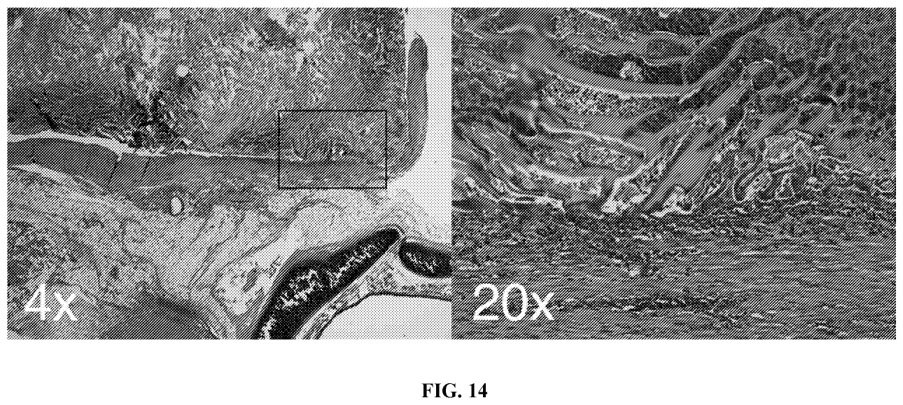

FIG. 14 shows histology of the resected rabbit trachea, 24 days after implantation. FIG. 14 at the 4× magnification shown in the left panel, shows that the histopathology at the interface of the bio-resorbable silk splint (identified with an asterisk) and underlying tracheal mucosa is well-delineated. FIG. 14 at the 20× objective shown in the right panel (corresponding to boxed area of the 4× left panel), shows that the histopathology exhibited a localized inflammatory reaction with early signs of fibrosis is evident (the arrow designates fibroblastic proliferation).

The anterior tracheal ring resection technique consistently produced significant airway malacia in our rabbit model. All three rabbits tolerated the procedure well, and neither respiratory distress nor other surgical complication was observed in the post-operative period.

The image-based assay of airway dynamic change with spontaneous breaths provided a reliable and quantitative metric for tracheomalacia in our model, allowing us to quantify both the severity of the surgically induced malacia, as well as the efficacy of the splint. This study, while relatively small, suggests that the current splint design yields a significant reduction in dynamic airway change in our model of severe malacia. Histology indicates an expected inflammatory response around the area of tissue injury at the site of the tracheal ring denudement from the underlying mucosa. At the 31-day time-point, there is indication of fibrosis at the interface of the splint ant tracheal mucosa. We hypothesize that this reactive fibrosis would subsequently lead to tissue integration with the splint matrix as the material breaks down, and that this is the principle mechanism for the progressive improvement in tracheal dynamics seen over time.

We developed a two-step method to fabricate silk fibroin-based splints that exhibited both flexibility and stiffness, allowing them to conform to the tracheal wall as well as provide radial strength and hold sutures. This fabrication method and the versatility of silk fibroin biopolymer will allow us to modulate the degradation rate of the devices by changing the secondary structure of the SF protein or the porosity of the material. Through an in vitro degradation assay, we were able to relate mass loss to a loss in force, and demonstrate that this relationship was linear. In future longitudinal studies, in vivo degradation estimated from histological images or explant weight changes could be compared to the in vitro degradation data to estimate the change in mechanical properties. Degradation in vivo was not observed over the time period in the present study; based on prior in vivo studies with silk-based devices it is expected to occur approximately 3-6 months post implantation. (See Thurber A E, et. al., In Vivo Bioresponses to Silk Proteins, 71 Biomaterials, 145-157 (2015)). Degradation rate has been shown to be dependent on the SF protein concentration, secondary structure, material density, and the in vivo location of the implant. (See Thurber A E, et. al., In Vivo Bioresponses to Silk Proteins, 71 Biomaterials, 145-157 (2015); see also Altman G H, et. al., Silk-Based Biomaterials, 24 Biomaterials 3, 401-416 (2003)).

Severe tracheomalacia is a serious condition and is not infrequent, accounting for almost half of all cases. It often requires surgical intervention and options are limited, especially when longer or multiple segments of the trachea are involved. External splints have been reported in both preclinical and human use, although these have been from non-degradable materials or degradable materials used off-label. These materials have not been developed for use in the context of the pediatric airway. As noted, the fabrication method of the SF splint will allow us to modulate the degradation rate and stiffness of the device.

This study of a resorbable silk fibroin splint suggested that the splint design and material were well tolerated and efficacious in a rabbit model of severe tracheomalacia, with marked reduction in airway collapse following implantation and good tissue-integration and tolerability over a clinically relevant time course.

References cited in the present disclosure are hereby incorporated by reference in their entirety. Other embodiments are within the scope and spirit of the disclosure. Features implementing functions may also be physically located at various positions, including being distributed such that portions of functions are implemented at different physical locations.

Further, the disclosure may include more than one invention.

### OTHER EMBODIMENTS AND EQUIVALENTS

While the present disclosure has explicitly discussed certain particular embodiments and examples of the present disclosure, those skilled in the art will appreciate that the disclosure is not intended to be limited to such embodiments or examples. On the contrary, the present disclosure encompasses various alternatives, modifications, and equivalents of such particular embodiments and/or example, as will be appreciated by those of skill in the art.

Accordingly, for example, methods and diagrams of should not be read as limited to a particular described order or arrangement of steps or elements unless explicitly stated or clearly required from context (e.g., otherwise inoperable). Furthermore, different features of particular elements that may be exemplified in different embodiments may be combined with one another in some embodiments.

What is claimed is:

1. A stent having a substantially cylindrical body,
wherein at least the body is comprised of a silk fibroin material characterized by beta-sheet secondary structure, and
wherein the stent is designed and engineered to be grafted to an external wall of a subject's trachea.

2. The stent of claim 1, wherein the silk fibroin material present in the body is formed from a silk fibroin solution having a concentration of about 1% w/w % to about 30% w/w %.

3. The stent of claim 1 or 2, wherein the silk fibroin material comprises an additive that is embedded within the material or coated on a surface of the body.

4. The stent of claim 3, wherein the additive is silk fibroin fibers.

5. The stent of claim 3, wherein the additive is a plasticizer.

6. The stent of claim 5, wherein the plasticizer is present in the silk fibroin material at a concentration of about 1% to about 30% by weight.

7. The stent of claim 5 or 6, wherein the plasticizer is selected from the group consisting of: 1,2-butylene glycol; 2-amino-2-methyl-1,3-propanediol; 2,3-butylene glycol; allyl glycolate; butyl lactate; diethanolamine; diethylene glycol monoethyl ether; ethyl glycolate; ethyl lactate; ethylene glycol; ethylene glycol monoethyl ether; glycerol; glyceryl monostearate; monoethanolamine; monisopropanolamine; monopropylene glycol monoisopropyl ether; polyethylene glycol; polyethylene oxides; propylene glycol; propylene glycol monoethyl ether; sorbitol lactate; styrene glycol; triethanolamine; triethylenetetramine; or combinations thereof.

8. The stent of any preceding claim, wherein the substantially cylindrical body is characterized by a radial opening between about 0° and about 240°.

9. The stent of any preceding claim, wherein the substantially cylindrical body has an elastic modulus of about 0.1 MPa to about 15 MPa.

10. The stent of any preceding claim, wherein the substantially cylindrical body has an average radial strength of about 50 mmHg to 500 mmHg.

11. The stent of any preceding claim, wherein the silk fibroin material is porous.

12. The stent of claim 3, wherein the additive is or comprises an active agent.

13. The stent of claim 12, wherein the active agent is or comprises a therapeutic.

14. The stent of any preceding claim, wherein viable cells are present in the silk fibroin material.

15. The stent of claim 3, wherein the additive is selected from the group consisting of antibodies or fragments or portions thereof antibiotics or antimicrobial compounds; antigens or epitopes; anti-proliferative agents; aptamers; biopolymers; cell adhesion proteins, cell attachment mediators; cleavable cross-linkers; cytokines; enzymes; growth factors or recombinant growth factors and fragments and variants thereof hormone antagonists; hormones; nanoparticles; nucleic acid analogs; nucleic acids; nucleotides; oligonucleotides; peptide nucleic acids (PNA); peptides; proteins; radiopaque markers; small molecules; soluble drugs, therapeutic agents and prodrugs; toxins; or combinations thereof.

16. The stent of any preceding claim, wherein the body programmably degrades.

17. The stent of claim 3, wherein the silk fibroin material is a blend of silk fibroin and a plasticizer having a ratio of between about 1000:1 to about 1:1 by dry weight.

18. The stent of claim 14, wherein the viable cells are patient derived cells.

19. The stent of any preceding claim, wherein the body is characterized by a tensile strength of about 1 MPa to about 15 MPa.

20. The stent of any preceding claim, comprising struts positioned on or within the silk fibroin material of the body.

21. The stent of claim 20, wherein the struts are silk-based fibers.

22. The stent of claim 20, wherein the struts are concentrated silk-based materials.

23. The stent of claim 20, wherein the struts are or comprise a metal.

24. The stent of claim 23, wherein the metal is or comprises magnesium.

25. The stent of claim 20, wherein the struts are or comprise a polymer.

26. The stent of claim 20, wherein the body is characterized by a tensile strength of about 1 MPa to about 15 MPa.

27. The stent of any preceding claim, wherein the stent is designed and arranged to receive sutures through the body or through holes in the body.

28. The stent of any preceding claim, further comprising barbs positioned along an outside of the body and arranged and constructed to prevent migration of the stent.

29. A method of manufacturing the tracheal stent of any preceding claim, the method comprising steps of:
providing a silk fibroin solution;
adding the solution to a mold; and
processing the solution to form the tracheal stent.

30. The method of claim 29, wherein the step of processing comprises freezing.

31. The method of claim 29, wherein the step of processing comprises porogen leaching.

32. The method of claim 29, wherein the step of processing comprises gel spinning.

33. The method of claim 29, wherein the step of processing comprises micromolding.

34. The method of claim 30, wherein the step of freezing comprises lowering a temperature of the solution to about −45° C. at a rate of about 0.1° C./minute to about 5° C./minute.

35. The method of claim 30, wherein the step of freezing comprises drying the solution under vacuum.

36. The method of any one of claims 29-35, further comprising a step of submerging the tracheal stent in methanol.

37. The method of any one of claims 29-36, further comprising a step of autoclaving the tracheal stent.

38. The method of any one of claims 29-37, further comprising a step of water annealing the tracheal stent.

39. The method of any one of claims 29-38, further comprising a step of encapsulating or embedding an additive in the silk fibroin solution, so that when the tracheal stent is formed the additive is embedded therein.

40. The method of any one of claims 29-39, further comprising a step of coating the tracheal stent with an additive.

41. The method of any one of claims 29-40, wherein the additive comprises an active agent, a plasticizer, silk fibroin fibers, a therapeutic, or combinations thereof.

42. The method of any one of claims 29-41, wherein the plasticizer is selected from the group consisting of: 1,2-butylene glycol; 2-amino-2-methyl-1,3-propanediol; 2,3-butylene glycol; allyl glycolate; butyl lactate; diethanolamine; diethylene glycol monoethyl ether; ethyl glycolate; ethyl lactate; ethylene glycol; ethylene glycol monoethyl ether; glycerol; glyceryl monostearate; monoethanolamine; monisopropanolamine; monopropylene glycol monoisopropyl ether; polyethylene glycol; polyethylene oxides; propylene glycol; propylene glycol monoethyl ether; sorbitol lactate; styrene glycol; triethanolamine; triethylenetetramine; or combinations thereof.

43. The method of any one of claims 29-40, wherein the additive comprises antibodies or fragments or portions thereof; antibiotics or antimicrobial compounds; antigens or epitopes; anti-proliferative agents; aptamers; biopolymers; cell adhesion proteins, cell attachment mediators; cleavable cross-linkers; cytokines; enzymes; growth factors or recombinant growth factors and fragments and variants thereof; hormone antagonists; hormones; nanoparticles; nucleic acid analogs; nucleic acids; nucleotides; oligonucleotides; peptide nucleic acids (PNA); peptides; proteins; radiopaque markers; small molecules; soluble drugs, therapeutic agents and prodrugs; toxins; or combinations thereof.

44. The method of any one of claims 29-43, further comprising encapsulating or embedding viable cells in the silk fibroin solution.

45. The method of claim 44, wherein the viable cells are patient derived cells.

46. A method of manufacturing the stent of any one of claims 1-27, the method comprising steps of:
providing a silk fibroin solution;
passing the silk fibroin solution through a 3D printer to generate the stent.

47. A method of installing a tracheal stent comprising grafting the stent of any one of claims 1-27 to an external site of a subject's trachea.

48. The stent of any one of claims 1-27, wherein in the stent graft is implantable in a body lumen, externally affixed to a tracheal wall for treatment of suprasomal collapse, tracheal malacia, or tracheal stenosis.

49. The stent of any one of claims 1-27, wherein the body has a length of about 0.5 cm to about 8 cm.

50. The stent of any one of claims 1-27, wherein the body has a thickness of about 1 mm to about 5 mm.

51. The stent of any one of claims 1-27, wherein the body has a radius of about 2.5 mm to about 10 mm.

52. A stent having a substantially cylindrical body,
wherein at least the body is comprised of a silk fibroin material characterized by beta-sheet secondary structure;
wherein the stent has a length of about 0.5 cm to about 8 cm, a thickness of about 1 mm to about 5 mm, and a radius of about 2.5 mm to about 10 mm,
wherein the body comprises a radial opening between about 0° and about 240°
wherein the stent is designed and engineered to be grafted to an external wall of a subject's trachea for treatment of suprasomal collapse, tracheal malacia, or tracheal stenosis.
